(19) United States
(12) Patent Application Publication
(10) Pub. No.: US 2018/0360845 A1
(43) Pub. Date: Dec. 20, 2018

(54) MUSCARINIC COMBINATION OF A SELECTIVE M2-ANTAGONIST AND A PERIPHERAL NON-SELECTIVE ANTAGONIST FOR TREATING HYPOCHOLINERGIC DISORDERS

(71) Applicant: CHASE PHARMACEUTICALS CORPORATION, Washington, DC (US)

(72) Inventors: Thomas N. CHASE, Washington, DC (US); Kathleen E. CLARENCE-SMITH, Washington, DC (US)

(73) Assignee: Chase Pharmaceuticals Corporation, Parsippany, NJ (US)

(21) Appl. No.: 15/746,092

(22) PCT Filed: Jul. 20, 2016
(86) PCT No.: PCT/US2016/043108

§ 371 Date: Jan. 19, 2018

Related U.S. Application Data
(60) Provisional application No. 62/194,527, filed on Jul. 20, 2015.
(60) Provisional application No. 62/298,206, filed on Feb. 22, 2016.
(60) Provisional application No. 62/360,004, filed on Jul. 8, 2016.

(51) Int. Cl. A61K 31/5513; A61P 25/28; A61P 25/18; A61K 45/06; A61K 31/4725

(52) U.S. Cl. CPC A61K 31/5513; A61P 25/28; A61K 31/4725; A61K 45/06; A61P 25/18

(57) ABSTRACT

A combination of a muscarinic receptor antagonist consisting of a M2-receptor antagonist and of a non-selective, peripheral anticholinergic agent, and optionally an anticholinesterase inhibitor, and use of the same for treatment of hypocholinergic type disorders such as Alzheimer type dementia, schizophrenia, schizophrenia associated dementia, and schizoaffective disorders.

### FIELD OF THE INVENTION

The invention pertains to the field of treating hypocholinergic disorders of the central nervous system, in particular of Alzheimer's Disease (AD)-type dementias, schizophrenia, schizophrenia associated dementia, Parkinson's dementia, Lewy body diseases, Down Syndrome, and chronic neuropathic pain, and provides a new combination of a cholinergic M₂-receptor antagonist agent and a cholinergic receptor antagonist, which optionally further includes an acetylcholinesterase inhibitor.

More particularly, the present invention relates to a new combination of a centrally active, selective-muscarinic M₂-receptor antagonist, herein below also referred to as “M₂-antagonist” or “M2-antagonist”, with a peripheral non-selective-muscarinic-receptor antagonist herein below also referred to as “non-selective Peripheral Anticholinergic Agent” (“nsPAChA”), as well as the optional addition of an acetyl choline esterase inhibitor (AChEI) to said M₂-antagonist/nsPAChA combination. This combination provides pro-cognitive activity by enabling safe administration of a M2-antagonist without inducing peripheral, dose-limiting adverse effects.

### Definitions

“AD”: Alzheimer's disease.
“CNS”: Central Nervous System.
“PNS”: Peripheral Nervous System.
“Muscarinic type receptors (mAChRs)”: Five subtypes of muscarinic receptors, M1 through M5, have been identified.
“ACh”: refers to the neurotransmitter acetylcholine.
“nsPAChA(s)”: non-selective, peripheral AntiCholinergic Agent(s) acting on the AChRs which are present in the PNS.
“Non-selective”: refers to nsPAChAs, and applies to muscarinic anticholinergic agents exhibiting inhibitory activity on the mAChRs broadly across the various subtypes of muscarinic M-receptors, namely the M₁-M₅ receptors.
“Selective”: refers to M₂-antagonists, and applies to antagonists of the mAChRs having an affinity for the M₂ receptor subtype higher than that for the M₁ and M₃-M₅ receptor subtypes, i.e. a muscarinic antagonist having a ratio of (Kᵢ for M₁ and, respectively, M₃-M₅/Kᵢ for M₂) greater than 1.
“Peripheral”: refers to muscarinic anticholinergic agents and applies to anticholinergics that are largely unable (have a limited ability) to enter the central nervous system following systemic administration and thus do not affect brain function to a clinically appreciable degree. These drugs can include both quaternary and tertiary ammonium anticholinergic agents, especially those having low lipid solubility.
“Anticholinergic therapy”: the treatment with an anticholinergic agent of such medical conditions as gastro-intestinal cramping, nausea, retching, vomiting, fecal incontinence, bladder spasms, urinary incontinence, overactive bladder, asthma, motion sickness, muscular spasms, and smooth muscle contractive disorders; or the treatment, if any, with an anticholinergic agent of side effects caused by cholinergic receptor agonists, including, but not limited to gastro-intestinal cramping, nausea, retching, vomiting, fecal incontinence, bladder spasms, urinary incontinence, overactive bladder, asthma, motion sickness, muscular spasms, and smooth muscle contractive disorders.
“CSF”: Cerebrospinal Fluid.
“IR”: Immediate Release of the active ingredient from a composition.
“ER”: Extended Release, including sustained release, controlled release and slow release of the active ingredient from a composition by any administration route, in particular, but not limited to oral and parenteral (including transcutaneous, transdermal, intramuscular, intravenous, and subcutaneous) routes.
“AChE”: Acetyl Choline Esterase.
“AChEI(s)”: Acetyl Choline Esterase Inhibitor(s).
“Transdermal delivery”: administration of drug via the skin which targets, without limitation, skin tissues just under the skin, other tissues or organs under the skin, systemic circulation, and/or the central nervous system.
“Transdermal Therapeutic System (TTS)”: administration of drug via transdermal delivery using transdermal drug formulations and transdermal patches incorporating such transdermal drug formulations.
A “maximum tolerated dose,” “maximal tolerated dose” or “MTD” refers to, and is defined as the highest dose of a drug or treatment that does not produce unacceptable side effects. The maximum tolerated dose is determined in clinical trials by testing increasing doses on different groups of people until the highest dose with acceptable side effects is found.
“comprising” means that the compositions and methods include the recited elements, but do not exclude others. “comprising” is inclusive of the terms “consisting of” and “consisting essentially of”.
“consisting essentially of” means that the methods and compositions may include additional steps, components or ingredients, but only if the additional steps, components or ingredients do not materially alter the basic and novel characteristics of the claimed methods and compositions. In certain embodiments, “consisting essentially of” means that the subsequently named component(s) is necessarily included but that another unlisted ingredient(s) that does not materially affect the basic and novel properties can also be present. For example, when used to define compositions and methods, “consisting essentially of” means excluding other elements of any essential significance to the combination for the intended use. Thus, for example, a composition consisting essentially of the elements as defined herein would not exclude trace contaminants and pharmaceutically acceptable carriers.
“pharmaceutically acceptable salt” means either a pharmaceutically acceptable acid addition salt or a pharmaceutically acceptable base addition salt of a currently disclosed compound that may be administered without any resultant substantial undesirable biological effect(s) or any resultant deleterious interaction(s) with any other component of a pharmaceutical composition in which it may be contained.
“hypocholinergic disorder” means a pathologic condition of the CNS due to or derived from a decrease in cholinergic transmission typically associated with events or diseases including but not limited to: Alzheimer disease, Alzheimer-type dementia, mild cognitive impairment, Lewy body disease dementia, Parkinson's disease dementia, post-stroke dementia, vascular dementia, traumatic brain injury, Down syndrome, anorexia nervosa, Tourette disease, tardive dyskinesia, Pick's disease, Huntington's chorea, Friedrich's ataxia, chronic neuropathic pain, falls, post-operative delirium, schizophrenia, schizophrenia associated dementia, and schizoaffective disorders.
“combination therapy” means treating a patient with a combination of a selective-muscarinic M₂-receptor antagonist that crosses the blood-brain barrier and a non-selective peripheral muscarinic receptor antagonist, and optionally further including an acetylcholinesterase inhibitor, as a therapeutic platform in a rotating, an alternating and/or a simultaneous treatment schedule or regimen. Combination therapy may include a temporal overlap of other therapeutic agents, depending on the clinical course of a given hypocholinergic disease in a subject.

### BACKGROUND OF THE INVENTION

Acetylcholinesterase inhibitors (AChEIs) currently constitute the major drug class used to treat AD type dementias. Medications of this type serve not only as part of the standard of care for patients suffering from a dementia of the AD type, but are also used off-label for various other, generally chronic and progressive, hypocholinergic CNS disorders. AChEIs have the enhancement of ACh-mediated neurotransmission as a general mechanism of action. All work to increase and prolong the availability of ACh in the brain by inhibiting its degradatory enzyme acetylcholinesterase. Four AChEIs have been approved by the U.S. F.D.A. for the treatment of dementias of the AD type: tacrine (now little used), donepezil [Aricept®], rivastigmine [Exelon®] and galantamine [Razadyne®]. Rivastigmine has also been approved for the treatment of Parkinson's disease dementia. AChEIs are available in various formulations including immediate release forms such as tablets, capsules and solutions as well as rapid dissolving and extended release forms for oral administration as well as those for parenteral (e.g. transdermal) administration.

Unfortunately, none of the AChEIs approved for use for AD type dementias provides more than modest symptomatic benefit to any of these disorders. A critical medical need thus exists to improve the efficacy of these cholinergic replacement therapies. Several alternative strategies exist to appropriately stimulate cholinergic transmission in the brain of those suffering from these hypocholinergic CNS disorders such as the AD type dementias.

Another approach to the treatment of AD type dementia is to inhibit presynaptic muscarinic receptors of the M₂ subtype by administering an antagonist selective for these receptors. M₂ muscarinic receptors are generously expressed on the presynaptic terminals of cholinergic neurons projecting to the hippocampus and cerebral cortex as well as in most other brain regions involved in learning and memory processes (Alcantara A A et al. 2001; and Rouse S T et al. 2000; the disclosures of which are incorporated herein in their entirety by reference). When stimulated by intrasynaptic ACh, these autoreceptors act to inhibit further ACh synthesis and release, thus attenuate cholinergic transmission (Tzavara E T et al. 2003; Lamping K G et al. 2004; and Zhang W et al. 2002; the disclosures of which are incorporated herein in their entirety by reference). Moreover, M₂ receptor knockout mice showed an impaired performance in passive avoidance testing that suggests a crucial role for these muscarinic receptors in the regulation not only of acetylcholine efflux but also of cognitive function (Tzavara E T et al. 2003). Similarly, drugs that block M₂ receptors increase ACh release and stimulate cholinergic transmission. For this reason, M₂ muscarinic receptor antagonists have been proposed as a potential cholinomimetic treatment of AD type dementia (Stoll C et al. 2009; the disclosure of which is incorporated herein in its entirety by reference).

However, after two decades of pharmacological and clinical studies, determining the potential efficacy in humans of selective M₂-antagonists remains an unsolved problem.

Numerous studies with a variety of M₂ receptor-preferring antagonists have been conducted to evaluate the role of M₂ muscarinic receptor blockade in the treatment of cognitive dysfunction. Most suggest that drugs acting to inhibit these receptors enhance learning and memory in the experimental animal. For example, treatment with the selective muscarinic M₂ antagonist, 5,11-dihydro-8-chloro-11-[[4-[3-[(2,2-dimethyl-1-oxopentyl)ethylamino]propyl]-1-piperidinyl]acetyl]-6H-pyrido[2,3-b][1,4]benzodiazepin-6-one (BIBN-99), reportedly reverses, in a dose-dependent manner, the impaired ACh release as well as the cognitive deficits observed in aged memory impaired rats. BIBN-99 also improves scopolamine-induced amnesia in young animals (Quirion R et al. 1995; the disclosure of which is incorporated herein in its entirety by reference). In traumatic brain-injured rats, BIBN 99 has been found to attenuate cognitive deficits (Pike B R et al. 1995; the disclosure of which is incorporated herein in its entirety by reference). The efficacy of BIBN-99 in these studies is presumed to relate to its antagonistic properties on negative muscarinic M₂ autoreceptors. Taken together, the forgoing observations could have implications for the treatment of degenerative disorders associated with impaired cholinergic function such as the AD type dementias (Rowe W B et al. 2003; the disclosure of which is incorporated herein in its entirety by reference).

Piperazine, piperazinyl-piperidine and piperidinyl-piperidine derivatives form a series of muscarinic M₂-antagonists studied for their pro-cognitive properties. For example, the muscarinic M₂ receptor antagonist 4-cyclohexyl-alpha-[4[[4-methoxyphenyl]sulphinyl]-phenyl]-1-piperazineacetonitrile (SCH 57790) produces a dose-related increase in brain acetylcholine release and enhances cognitive performance in rodents and nonhuman primates, effects that were qualitatively similar to those produced by the AChEI donepezil. The forgoing results support the view that blockade of muscarinic M₂ receptors is a viable approach to enhancing cognitive performance (Carey G J et al. 2001; the disclosure of which is incorporated herein in its entirety by reference).

Similarly, the piperazinyl-piperidine derivative 4-[4-[1(S)-[4-[(1,3-benzodioxol-5-yl) sulfonyl]phenyl]ethyl]-3(R)-methyl-1-piperazinyl]-4-methyl-1-(propylsulfonyl)piperidine (SCH 72788), has also been found to be a functional M₂ receptor antagonist that increases ACh release and is active in a rodent model of cognition, suggesting that M₂ receptor antagonists may be useful for treating the cognitive decline observed in AD and other dementias (Lachowicz J E et al. 2001; the disclosure of which is incorporated herein in its entirety by reference).

The piperidinyl-piperidine derivative SCH-217443, a potent and selective M₂ antagonist, shows activity in a rat model of cognition (Greenlee W et al. 2001; the disclosure of which is incorporated herein in its entirety by reference).

Methoctramine, a polymethylenetetraamine, acts as a muscarinic antagonist that binds preferently to the pre-synaptic M₂ receptor. In female rats, the bilateral intrastriatal infusion of methoctramine improves procedural memory performance presumably by enhancing the release of acetylcholine (Lazaris A Et al. 2003; the disclosure of which is incorporated herein in its entirety by reference).

Other drugs, identified as having relatively selective M₂ muscarinic receptor antagonist activity but receiving little or no investigative attention in preclinical cognitive models, include: otenzepad (AF-DX 116), its (+)-enantiomer (AF-DX 250) and its analog AF-DX 384, (±)-5,11-dihydro-11-([(2-[(dipropylamino)methyl]-1-piperidinyl)ethyl)amino]carbonyl)-6H-pyrido(2,3-b)(1,4)benzodiazepine-6-one; caproctamine; and benextramine.

Among these M₂-antagonists, otenzepad only is reputed to be administered to human beings. Studies in human volunteers, reviewed in Adis Drug R&D 1999, the disclosure of which is incorporated herein in its entirety by reference, showed that this drug:
may be orally administered to a human being at a dose of 120 mg bid or 240 mg bid;
does not induce manifest muscarinic adverse effects after single oral doses of 120, 240 and 480 mg; and
gives mild and tolerable adverse events, including palpitations, dry mouth and headache in about 20% of the subjects, when administered at 270 to 810 mg/day,
   but no other information of a clinical nature concerning otenzepad appears in the subsequent literature, specifically, no information concerning the adequacy of its cognitive efficacy at the doses administered to patients with hypocholinergic type dementias is found in the literature.

Thus, notwithstanding the intense and protracted preclinical focus on studies of the relation between presynaptic M₂ receptor blockade and cognitive function, there have been no (known published) favorable results of therapeutic trials of highly selective M₂ antagonists in patients with AD type dementia. On the other hand, several pharmaceuticals with M₂ receptor antagonist activity and other pharmacologic activities, have been evaluated, some as potential candidates for the treatment of dementia. These potential candidates are briefly illustrated herein below.
Lu 25-109—The arecoline bioisostere, 5-(2-ethyl-2H-tetrazol-5-yl)-1-methyl-1,2, 3,6-tetrahydropyridine (INN alvameline), has been under development for the treatment of AD type dementia. In a clinical study (Sramek J J et al. 1998), the fixed-dose Maximum Tolerated Dose of LU-25-109 was established to be 150 mg t.i.d. In binding assays, alvameline acts as a partial M₁ agonist and as a M₃ and mainly M₂ antagonist with low affinities for other receptor types but is extensively metabolized in rodents and humans (Christensen E B et al. 1999). To evaluate the therapeutic effect of LU 25-109, a 6-month, randomized, double-blind, placebo-controlled trial comparing three doses of Lu25-109 with placebo was carried out in 496 patients with probable AD. There were no significant differences for either of the two primary or the secondary variables, but a trend was observed for patients on the highest drug dose to worsen in the completer's analysis. This lack of antidementia efficacy may be partially attributable to the fact that Lu 25-109, while inhibiting presynaptic M₂ muscarinic receptors, also blocks postsynaptic M₁ and M₃ receptors. Additionally, the drug produced prominent cholinergic side effects, including anorexia, nausea, diarrhea and increased sweating, all of which increased with increasing drug dose, thus preventing the administration of doses sufficient to benefit cognition (Thal L J et al. 2000, the disclosure of which is incorporated herein in its entirety by reference). Thus, this document does not give any report of the efficacy of alvameline in humans; on the contrary, it reports a lack of antidementia efficacy at the doses used, which were just below those maximally tolerated by humans.
Dimethindene, also known as dimetindene (trade name Fenistil), is N,N-Dimethyl-3-[1-(2-pyridinyl)ethyl]-1H-indene-2-ethanamine, CAS 0005636-83-9. It can be prepared as described by Huebner et al, in U.S. Pat. No. 2,970,149, which also describes the optically active form of 2-(2-dimethylamino-ethyl))-3-[1-(2-pyridyl)-ethyl]-indene with an [α]_{D}^{25°} of +70, or in J Am Chem Soc 1960, 87, 2077; the disclosure of which is incorporated herein in its entirety by reference. Dimet(h)indene is sold OTC in a number of countries worldwide as orally or locally administered antipruritic. Dimethindene for oral administration is available for example in IR coated tablets containing 1 mg dimet(h)indene or in ER unit forms containing 4 mg dimet(h)indene and the daily recommended dose is from 3 to 6 mg. The (S)-(+)-dimethindene, N,N-Dimethyl-3-[(1S)-1-(2-pyridinyl)ethyl]-1H-indene-2-ethanamine, is a potent M₂-selective muscarinic receptor antagonist (Pfaff et al. 1995; the disclosure of which is incorporated herein in its entirety by reference). The (R)-(−)-enantiomer is the eutomer (responsible for bioactivity) for histamine H₁ receptor binding (Histamine H₁-receptor antagonist).
Tripitramine, 11-[2-[6-[8-[6-[bis[2-oxo-2-(6-oxo-5H-pyrido[2,3-b][1,4]benzodiazepin-11-yl)ethyl]amino]hexyl-methylamino]octyl-methylamino]hexylamino]acetyl]-5H-pyrido[2,3-b][1,4]benzodiazepin-6-one, is a selective M₂ cholinergic antagonist. Binding selectivity results indicate that it binds to the muscarinic M₂ receptor with a Kᵢ value of 0.27+/−0.02 nM (Maggio R et al. 1994; the disclosure of which is incorporated herein in its entirety by reference). Tripitramine can be prepared as described by Melchiorre et al. 1993; the disclosure of which is incorporated herein in its entirety by reference).
Himbacine (3aR,4R,4aS,8aR,9aS)-4-{(E)-[(2R,6S)-1,6-dimethylpiperidin-2-yl]vinyl}-3-methyldecahydronaphtho[2,3-c]furan-1(3H)-one, an alkaloid isolated from the bark of Australian magnolia, is a potent muscarinic receptor antagonist that displays selectivity for the M₂ and M₄ receptors but failed in clinical development.
(+)-Himbacine was obtained by total synthesis (S. Chackalamannil et al. 1999; the disclosure of which is incorporated herein in its entirety by reference.).
The “himbacine analog” (3aR,4R,4aS,8aR,9aS)-4-{(E)-[(2R,6S)-1,6-dimethylpiperidin-2-yl]ethynyl}-3-methyldecahydronaphtho [2,3-c]furan-1(3H)-one, differing from himbacine by the presence of a triple bond, instead of a double bond, in its side chain, was reported to be a M₂ antagonist, but exhibited lower affinity and showed a 10-fold selectivity for the M₂/M₃ receptors (Gao et al. 2006; the disclosure of which are incorporated herein in their entirety by reference)—
AQ-RA741, -11-([4-[4-(Diethylamino)butyl]-1-piperidinyl]acetyl)-5,11-dihydro-6H-pyrido[2,3-b][1,4]benzodiazepin-6-one, is another otenzepad analog disclosed as a selective, in particular cardioselective, high affinity M₂ muscarinic receptor antagonist. Its development by Boehringer Ingelheim Pharma KG was discontinued due to adverse effects including cardiac arrhythmias (ADIS Insight, “at a glance”: discontinued in 1997—http: at adisinsight.springer.com/drugs/800002261; the disclosure of which is incorporated herein in its entirety by reference).

U.S. Pat. No. 5,952,349 (Asberom et al), the disclosure of which is incorporated herein in its entirety by reference, discloses 1,4-di-substituted piperidines wherein the position 4 bears an optionally substituted anilino-group and the position 1 bears a hydrogen atom or a functional group. These compounds, alone or in combination with acetylcholinesterase inhibitors, are considered useful in the treatment of cognitive disorders.

U.S. Pat. No. 5,883,096, U.S. Pat. No. 6,037,352, U.S. Pat. No. 6,288,068 and U.S. Pat. No. 6,498,168 (Lowe et al. —see also WO 96/26196), the disclosures of which are incorporated herein in their entirety by reference, disclose di-N-substituted piperazines and 1,4-di-substituted piperidines useful in the treatment of cognitive disorders, pharmaceutical compositions containing said compounds, methods of treatment using compounds, in particular for the treatment of Alzheimer-type dementia; and the use of said compounds in combination with acetylcholinesterase inhibitors. In particular, these di-N-substituted piperazines and 1,4-di-substituted piperidines are endowed with selective M₂ and/or M₄ muscarinic antagonizing activity.

U.S. Pat. No. 5,935,958 (Kozlowski et al.—see also WO 98/00412), the disclosure of which is incorporated herein in its entirety by reference, discloses di-N-substituted piperazines and 1,4-di-substituted piperidines linked, via a nitrogen atom of said piperazine or the nitrogen atom of said piperidine, to a bicyclic structure. Said compounds are preferably M₂ or M₄ selective muscarinic antagonists. This document also discloses combinations of said compounds with an AChEI.

WO 00/00488 (Kozlowski et al.), the disclosure of which is incorporated herein in its entirety by reference, discloses di-N-substituted piperazines and 1,4-di-substituted piperidines linked to a pyridine, pyrazine or thiophene group via a substituted methyl group. These compounds are muscarinic agonists useful in the treatment of cognitive disorders, pharmaceutical compositions containing the compounds, methods of treatment using the compounds, and to the use of said compounds in combination with acetylcholinesterase inhibitors.

U.S. Pat. No. 6,294,554 (Clader et al.), the disclosure of which is incorporated herein in its entirety by reference, discloses diphenyl-sulfones and phenyl-sulfonyl pyridines linked to a bipiperidine moiety via an optionally substituted methyl group, said substitution also including an ethylenedioxy group. These compounds are endowed with a selective M₂-antagonist activity. This document specifically describes the 1′-(2-amino-3-methylbenzoyl)-4-[[[(3-chlorophenyl)sulfonyl]phenyl]methyl]-1,4′-bipiperidine, also known as SCH-211803, and the 1′-(2-amino-3-methylbenzoyl)-4-[[[(3-chlorophenyl)sulfonyl]phenyl]ethylenedioxymethyl]-1,4′-bipiperidine, also known as SCH-217443.

A selective M₂ receptor antagonist with a bipiperidine moiety, showing superior M₂ receptor selectivity profile over SCH 211803, is “Compound 30” of formula

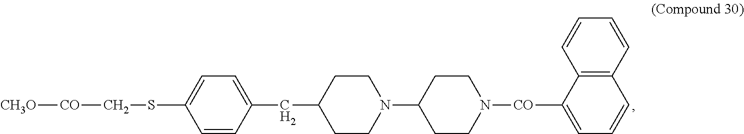

herein below referred to as “Wang Compound 30”, disclosed by Y. Wang et al. 2002, the disclosure of which is incorporated herein in its entirety by reference.

Other bipiperidine derivatives showing highly potent and selective M₂ receptor antagonist activity include “Compound 19” of formula

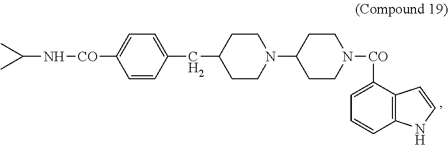 [SMILES: CC(C)NC(=O)c1ccc(CC2CCN(C3CCN(C(=O)c4cccc5[nH]ccc45)CC3)CC2)cc1]

herein below referred to as “Palani Compound 19”, and its analog “Compound 31”, of formula

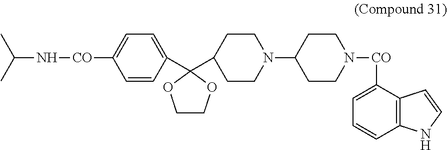 [SMILES: CC(C)NC(=O)c1ccc(C2(C3CCN(C4CCN(C(=O)c5cccc6[nH]ccc56)CC4)CC3)OCCO2)cc1]

herein below referred to as “Palani Compound 31”, disclosed by A. Palani et al. 2004, the disclosure of which is incorporated herein in their entirety by reference.

U.S. Pat. No. 6,458,812 (McKittrick et al.—see also WO 02/051808), the disclosure of which is incorporated herein in its entirety by reference, discloses amide derivatives of 1,4-di-substituted piperidines wherein the most represented substituent in position 4 is a cycloalkyl group. Also these compounds are selective M₂ or M₄ muscarinic antagonists that may be used, alone or in combination with an acetylcholinesterase inhibitor, for treating a cognitive or neurodegenerative disease.

U.S. Pat. No. 6,906,081 (Hey et al.—see also WO 02/072093), the disclosure of which is incorporated herein in its entirety by reference, discloses the use of a dual histamine H3 receptor antagonist/M₂ muscarinic antagonist, or a combination of an histamine H3 receptor antagonist with a M₂ muscarinic antagonist, for the treatment of cognition deficit disorders, in particular Alzheimer's type dementia. This document also discloses said dual 5HT3-antagonist/M₂-antagonist medicament used in combination with an AChEI.

WO 03/031412 (Wang et al.—see also EP 1434766 and US 2003/0207917), the disclosure of which is incorporated herein in its entirety by reference, discloses piperidine compounds as muscarinic receptor antagonists, as well as methods for preparing such compounds. In another embodiment, this document discloses pharmaceutical compositions comprising such muscarinic receptor antagonists as well as methods for using them to treat cognitive disorders such as Alzheimer's disease.

U.S. Pat. No. 6,890,936 (Boyle et al.—see also WO 03/091220), the disclosure of which is incorporated herein in its entirety by reference, discloses certain 6-aza-spiro[2,3]octanes as antagonists of the muscarinic receptors (M₂ and/or M₄) and methods of treatment, prevention or amelioration of one or more diseases associated with the muscarinic receptors also in combination with at least one acetylcholinesterase inhibitor.

U.S. Pat. No. 7,361,668 (Guyaux et al.), the disclosure of which is incorporated herein in its entirety by reference, discloses alkyne-quinuclidines that are endowed with a potent M₂ and/or M₃ muscarinic-antagonist activity and show pIC₅₀ values ranging from 7 to 10 for the M₃ and/or the M₂ receptors in the binding tests. However, these compounds have been mainly considered as M₃-receptor antagonists (J.-P. Starck et al. 2006, the disclosure of which is incorporated herein in its entirety by reference).

It is also well documented that schizophrenic patients experience cognitive disturbances that are not well addressed by current medications (Foster D J et al. 2012 December; 14(79):413-20).

An improvement in the treatment of the cognitive disorders globally designated as hypocholinergic disorders of the brain including dementias of Alzheimer's type, is possible by combining an AChEI with a nsPAChA (U.S. Pat. No. 8,404,701, the disclosure of which is incorporated herein in its entirety by reference) or with a non-anticholinergic antiemetic agent (U.S. Pat. No. 8,877,768, the disclosure of which is incorporated herein in its entirety by reference).

In summary, despite an interest in (see Brashear 1996), and the extensive studies conducted on a series of M₂-antagonist compounds, none of these compounds showed efficacy in humans at safe and tolerable doses and, conversely, said compounds induced adverse effects that were dose-limiting.

### SUMMARY OF THE INVENTION

The present inventors recognized the critical need to take advantage of the potent activity of a M2 muscarinic antagonist for the treatment of hypocholinergic disorders as defined above, in particular AD-type dementia, schizophrenia, and schizophrenia associated.

The present invention relates to a pharmaceutical combination comprising:
(a) a muscarinic receptor antagonist selected from the group consisting of centrally active, selective M₂-muscarinic cholinergic receptor antagonists (M₂-antagonist); and
(b) a muscarinic receptor antagonist selected from the group consisting of non-selective, peripheral anticholinergic agents (nsPAChAs); and, optionally,
(c) an acetyl choline esterase inhibitor (AChEI).

The M₂-antagonist Component (a) in the combination of the present invention may be selected from the group consisting of:
5-(2-ethyl-2H-tetrazol-5-yl)-1-methyl-1,2, 3,6-tetrahydropyridine (alvameline)
5,11-dihydro-8-chloro-11-[[4-[3-[(2,2-dimethyl-1-oxopentyl)ethylamino]propyl]-1-piperidinyl]acetyl]-6H-pyrido [2,3-b][1,4]benzodiazepin-6-one (BIBN-99);
racemic 11-[[2-(Diethylamino)methyl]-1-piperidinyl]-acetyl]-5,11-dihydro-6H-pyrido[2,3-b][1,4]benzodiazepin-6-one (otenzepad);
dextrorotatory 11-[[2-(diethylamino)methyl]-1-piperidinyl]-acetyl]-5,11-dihydro-6H-pyrido[2,3-b]E1,4]benzodiazepin-6-one [(+)-otenzepad];
N-2-[2-[(dipropylamino)methyl]-1-piperidinyl]ethyl]-5-,6-dihydro-11-H-pyrido[2,3-b][1,4]benzodiazepine-11-carboxamide (AF-DX 384);
11-[[4-[4-(Diethylamino)butyl]-1-piperidinyl]acetyl]-5,11-dihydro-6H-pyrido[2,3-b][1,4]benzodiazepin-6-one (AQ-RA 741);
N,N-Dimethyl-3-[1-(2-pyridinyl)ethyl]-1H-indene-2-ethanamine (dimethindene);
N,N-Dimethyl-3-[(1S)-1-(2-pyridinyl)ethyl]-1H-indene-2-ethanamine [S-(+)-dimethindene];
N,N′-bis[6-[([2-methoxyphenyl)methyl]amino]hexyl]-1,8-octanediamine (methoctramine);
1,1,24-tris[[5,11-dihydro-6-oxo-6H-pyrido[2,3b][1,4]-benzodiazepin-11-yl)carbonyl] methyl]-8,17-dimethyl-1,8,17,24-tetraazatetracosane (tripitramine);
(3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine;
(3S,3aR,4R,4aS,8aR,9aS)-3-Methyl-4-[2-((R)-1-methyl-6-(S)-methyl-piperidin-2-yl)-vinyl]-decahydro-naphtho [2,3-c]furan-1-one [(+)-himbacine];
(3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog);
4-cyclohexyl-alpha-[4[[4-methoxyphenyl]sulphinyl]-phenyl]-1-piperazineacetonitrile (SCH-57790);
4-[4-[1(S)-[4-[(1,3-benzodioxol-5-yl)sulfonyl]phenyl]ethyl]-3(R)-methyl-1-piperazinyl]-4-methyl-1-(propylsulfonyl)piperidine (SCH-72788);
1′-(2-methylbenzoyl)-4-[[[(3,4-methylenedioxyphenyl)sulfonyl]phenyl]methyl]-1,4′-bipiperidine (SCH-76050);
1′-(2-amino-3-methylbenzoyl)-4-[[[(3-chlorophenyl) sulfonyl]phenyl]methyl]-1,4′-bipiperidine (SCH-211803);
1′-(2-amino-3-methylbenzoyl)-4-[[[(3-chlorophenyl) sulfonyl]phenyl]ethylenedioxy methyl]-1,4′-bipiperidine (SCH-217443);
1′-naphto-1-yl-4-[[4-[(methoxycarbonyl)methylthio]phenyl]methyl]-1,4′-bipiperidine (Wang Compound 30);
1′-(indol-4-yl)carbonyl-4-[[(4-isopropyl)carbonyl]phenyl]methyl]-1,4′-biperidine (Palani Compound 19);
1′-(indol-4-yl)carbonyl-4-[[(4-isopropyl)carbonyl]phenyl]ethylenedioxymethyl]-1,4′-biperidine (Palani Compound 30);
   and pharmaceutically acceptable salts and solvates thereof.

In one embodiment, Component (a) is present in an amount from 0.5 mg to 1500 mg.

The nsPAChA Component (b) in the combination of the present invention may be selected from the group consisting of quaternary ammonium nsPAChAs, sulfonium nsPAChAs, (1S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)—N,N-diisopropyl-3-(2-hydroxy-5-methylphenyl)-3-phenylpropanamine (tolterodine) and its pharmaceutically acceptable salts, [2-[(1R)-3-(di(propan-2-yl)amino)-1-phenylpropyl]-4-(hydroxymethyl)phenyl] 2-methylpropanoate (fesoterodine) and its pharmaceutically acceptable salts; and 4-diethylaminobut-2-ynyl 2-cyclohexyl-2-hydroxy-2-phenylethanoate (oxybutynin) and its pharmaceutically acceptable salts, in a TTS (“TTS-oxybutynin”).

In one particular embodiment, the quaternary ammonium nsPAChAs or sulfonium nsPAChAs in the combination of the present invention have the formula (I)

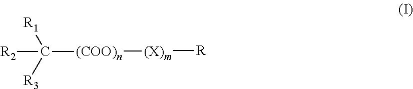

wherein
R is a radical selected from the group consisting of those of formulas (a)-(e)

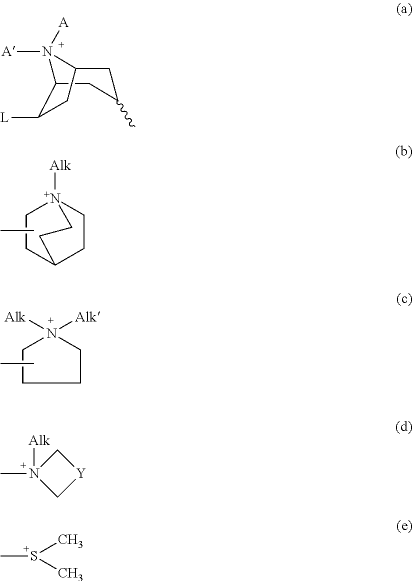

A being methyl and A′ being (C₁-C₄)alkyl or 2-fluoroethyl group or A and A′ forming a 1,4-butylene or 1,5-pentylene chain, L being hydrogen or methoxy, Alk and Alk′ each being (C₁-C₄)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion;
n and m, independently, are zero or 1;
X is a (C₂-C₃)alkylene group;
R₁ and R₂ are each phenyl, cyclopentyl, cyclohexyl, 1-cyclohexenyl, 2-thienyl and, when R is a radical (a), also each represents (C₁-C₄)alkyl;
R₃ is H or OH or, only when R is a radical (a), also a COOAlk group, Alk being a (C₁-C₄)alkyl group.

Preferably, according to this particular embodiment, in the above formula I at least one of m and n is 1.

In another embodiment, the nsPAChA Component (b) of the combination of the present invention is selected from the group consisting of azoniaspiro[33-benziloyloxy-(1α,5α)-nortropane-8,1′-pyrrolidine] (trospium) chloride, 3-[2-cyclopentyl(hydroxy)phenylacetoxy]-1,1-dimethylpyrrolidinium (glycopyrronium) bromide, solifenacin and the compound thereof with succinic acid (solifenacin succinate), propiverine and the hydrochloride thereof, oxyphencyclimine and the hydrochloride thereof, tolterodine and the hydrogen tartrate thereof, fesoterodine and the fumarate thereof; and TTS-oxybutynin, wherein said TTS is a transdermal patch.

In a preferred embodiment, in said combination the M2 antagonist Component (a) is selected from the group consisting of alvameline and pharmaceutically acceptable salts thereof; and the nsPAChA Component (b) is selected from the group consisting of oxybutynin and pharmaceutically acceptable salts thereof in a TTS. Preferably, according to this embodiment said nsPAChA Component (b) is oxybutynin in a transdermal patch releasing from 3.9 mg/24 to 7.8 mg/24 h oxybutynin.

In one embodiment, the M₂ antagonist Component (a) is formulated in a pharmaceutical composition or device in admixture with a pharmaceutical carrier or vehicle.

In another embodiment, the composition or device also includes the nsPAChA Component (b).

Thus, the present invention also provides the above combination, wherein said Components (a) and (b) are formulated in the same unit form. Herein below, the (a)+(b) fixed-dose combination will also be designated as “Component (a/b)”.

According to the present invention, the novel pharmaceutical composition in dosage unit form comprising, as active ingredients, (a) a M₂-antagonist and (b) a nsPAChA, in admixture with a pharmaceutical carrier or vehicle, is a particularly advantageous embodiment of the present invention.

The present invention also provides the addition of an AChEI to the above M₂-antagonist/nsPAChA combination, thus assuring a maximum supply of acetylcholine to the CNS by the administration of a combination of the three components. Herein below, the third AChEI component will also be designated as “Component (c). When Components (b) and Component (c) are formulated in the same unit form, said fixed-dose combination will also be designated as “Component (b/c)”.

The combination of the present invention allows the administration of a M₂-antagonist at even high doses, never previously administered to a human being, without any sign of adverse effects thus, on one side, eliminating the dose-limit that heretofore did not permit the full expression of the M₂-antagonists' potency and, on the other side, allowing the treatment with said M₂-antagonists of patients suffering from hypocholinergic disorders such as Alzheimer type dementia.

Thus, the present invention provides a combination of a M₂-antagonist and of a non-selective, peripheral anticholinergic agent (nsPAChA), and, optionally, an AChEI, for use in the treatment of a hypocholinergic disorder.

The present invention also relates to a method of treating a hypocholinergic type dementia comprising administering to a patient in need thereof, an effective dose of a M₂-antagonist, in combination with a non-selective, peripheral muscarinic anticholinergic agent (nsPAChA) and, optionally, an AChEI.

In a particular embodiment, the hypocholinergic type dementia is Alzheimer type dementia or schizophrenia.

The method or the use according to the present invention also involves treating a hypocholinergic disorder comprising administering to a patient in need thereof, an effective dose of a M₂-antagonist, in combination with a non-selective, peripheral muscarinic anticholinergic agent (nsPAChA).

The hypocholinergic disorders of the CNS are those indicated in the above “Definitions”.

According to an embodiment, the hypocholinergic disorder is selected from the group consisting of schizophrenia, schizophrenia associated dementia, and schizoaffective disorders.

In a particular embodiment, the hypocholinergic disorder is Alzheimer type dementia.

### DETAILED DESCRIPTION

Reduced levels of neurotransmitters including acetylcholine occur in dementias such as the Alzheimer disease (AD) type, and reduced cholinergic transmission is thought to attend the cognitive deficits observed in schizophrenia, and schizophrenia-associated dementia. In particular, a deficit in acetylcholine (ACh)-mediated transmission is thought to contribute to the cognitive and neurobehavioral abnormalities associated with these disorders. Accordingly, drugs known to augment cholinergic transmission in the Central Nervous System (CNS) are the mainstay of current therapy for AD. In addition, other diseases of the nervous system also involve decreased cholinergic transmission and, together with AD type disorders, are referred to as hypocholinergic disorders of the CNS, as defined above.

The present invention provides a combination of two muscarinic antagonists with different targets.

The present invention provides a treatment based on selective M₂ antagonism at doses that could not previously be achieved due to adverse effects. There have been no published accounts of clinical studies demonstrating the ability of selective M₂ muscarinic antagonists to treat hypocholinergic disorders of the CNS such as dementia of the AD type and schizophrenia or schizophrenia associated dementia. A major deterrent to such clinical investigations, as described above, is that treatment with muscarinic agonists and/or antagonists has been limited by dose-limiting side effects, especially those reflecting hyperstimulation of peripheral muscarinic receptors.

It is asserted that the lack of efficacy of the tested M₂-antagonists cannot be explained by an intrinsic inactivity of said compounds in humans but instead is a result of insufficient concentrations of M₂-antagonist in the CNS due to the occurrence of dose-limiting side effects. Consequently, a major cause of failure of such clinical investigations, as described above, was that treatment with muscarinic agonists and/or antagonists has been limited by dose-limiting side effects in the periphery (i.e., outside the CNS), especially those reflecting hyperstimulation of peripheral muscarinic receptors.

It has been unexpectedly found that, by the combination of two muscarinic antagonists, one being a selective muscarinic M₂-antagonist that crosses the Blood Brain Barrier (BBB) and the other being a non-selective, peripheral muscarinic antagonist, it is possible not only to safely administer said M₂-antagonist at the previously non-tolerated doses, but also to administer even high doses of said M₂-antagonist, thus allowing the treatment of the aforementioned hypocholinergic disorders and enabling greater efficacy.

Specifically, previous studies with M₂ antagonists do not mention a possible combination of M₂ antagonists with non-selective peripheral muscarinic antagonists (nsPAChAs), nor do they mention that the administration of such a combination can enable the safe and tolerable administration of doses of M₂-antagonists capable of further potentiating central cholinergic transmission so as to achieve efficacy in those suffering from hypocholinergic disorders, such as AD type dementias and schizophrenia or schizophrenia associated dementia, as provided in the present invention.

None of the aforementioned documents proposed a M₂ muscarinic antagonist drug acting both centrally in the brain and peripherally in combination with another muscarinic antagonist drug acting peripherally, to inhibit the peripherally mediated cholinergic dose-limiting side effects, in order to safely and tolerably administer M₂-antagonist at doses that heretofore caused intolerable adverse effects, or the administration of effective higher M₂-antagonist doses, for the greater benefit of those with hypocholinergic dementias of the AD type. In summary, none of the approaches in the art, as discussed herein, offered a clinical benefit to patients suffering from AD type dementias. Indeed, at present, only the AChEIs have been approved for the treatment of AD type dementia. On average, said AChEIs confer no more than 3 points of cognitive improvement on the standard 70 point ADAS-cog dementia scale, a degree of benefit that is generally of little functional significance (Birks J 2006).

More particularly, the present invention provides the combination of a centrally acting, selective muscarinic-M₂-antagonist drug and a non-selective, peripheral-muscarinic-antagonist drug (nsPAChA) for the safe treatment of hypocholinergic disorders such as dementia of Alzheimer type, schizophrenia, schizophrenia-associated dementia, and other hypocholinergic type cognitive or behavioral disorders. The muscarinic-M₂-antagonist in combination with a nsPAChA provides pro-cognitive properties for the treatment of such disorders without appreciable involvement of the PNS.

The present invention thus enables the heretofore inapplicable, safe and tolerable use of a presynaptic M₂ muscarinic receptor antagonist in the treatment of hypocholinergic disorders such as Alzheimer, dementia of Alzheimer type, schizophrenia, schizophrenia associated dementia, and other hypocholinergic type cognitive or behavioral disorders, such those described above, by combining said M₂-antagonist with a nsPAChA. This unlikely combination of two muscarinic antagonists, allows M₂-antagonists to be safely used at doses higher than those reportedly tolerated in clinical studies, in order to allow therapeutically adequate blockade of M₂-receptors in the CNS with attending safe increase in acetylcholine presynaptic release.

In addition, the present inventors have found that a combination of M₂-antagonists with a nsPAChA surprisingly acts to attenuate the dose-limiting side effects of M₂-antagonists; thus enabling a greater increase in the MTD of M₂-antagonists and enabling full efficacy or greater efficacy for the treatment of hypocholinergic disorders of the brain, notably AD, AD type dementia, schizophrenia, and schizophrenia associated dementia.

Thus, it has been found that a combination of M₂-antagonists with a nsPAChA allows for the safe administration of M₂-antagonists at doses never safely attained heretofore. In particular, a nsPAChA when concurrently or sequentially administered in combination with a M₂-antagonist, is able not only to neutralize the dose-limiting adverse effects that hindered the development of a M₂-antagonist for the treatment of central disorders due to a deficit of acetylcholine in the brain, but also to increase the supply of ACh at the cholinergic synapse in the CNS and thus increase cholinergic transmission in the CNS.

Thus, the use of a nsPAChA in combination with a M₂-antagonist that acts both centrally (in the brain) and peripherally (outside the brain), enables the safe administration of a M₂-antagonist at previously intolerable doses or at even higher doses of a M₂-antagonist, and allows for the treatment of a patient suffering from hypocholinergic disorders of the central nervous system, including but not limited to, AD, AD-type dementia, schizophrenia, schizophreniform conditions, schizophrenia associated dementia, schizoaffective disorders, Mild Cognitive Impairment (MCI), Lewy Body Disease dementia (LBD), Frontotemporal degeneration, Parkinson disease dementia (PDD), post-stroke dementia, vascular dementia, Traumatic Brain Injury, Anorexia Nervosa, Down syndrome, Tourette syndrome, tardive dyskinesia, Pick's disease, Huntington's chorea, Friedrich's ataxia, post-operative delirium, and falls. A combination as described herein allows a M₂-antagonist to safely increase the acetylcholine presynaptic release in the CNS and to improve cognition.

The finding of the present invention eliminates the adverse event-imposed dose-limit that, in the past, caused the failure of all clinical trials to demonstrate efficacy, thus providing a method for treating Alzheimer type dementia as well as central hypocholinergic disorders of the CNS by enabling the full efficacy of M₂-antagonists.

The present invention provides a composition useful for treating a patient with a hypocholinergic disorder as described herein, which comprises a nsPAChA in combination with a M₂-antagonist. Preferably, the dose of nsPAChA is at a dose higher than that used in anticholinergic therapy.

The present invention provides said pharmaceutical combination or composition comprising a M₂-antagonist and a nsPAChA for use for combating a hypocholinergic disorder in a patient.

The present invention also provides the addition of an AChEI to the above combination, thus assuring a maximum supply of acetylcholine to the CNS by the administration of a triple combination.

The present invention provides a method of treating a patient with a hypocholinergic disorder of the brain as described herein, which comprises treating such patient in need of treatment with a nsPAChA in combination with a M₂-antagonist. This treatment method precludes the onset of M₂-antagonist-associated peripheral dose-limiting adverse effects as well as the onset of nsPAChA central adverse effects, because these anticholinergics are substantially peripheral.

In another embodiment, the method of the present invention may further include administration of an AChEI.

Thus, the present invention provides a pharmaceutical combination comprising as Components:
(a) a muscarinic receptor antagonist selected from the group consisting of selective M₂-antagonists (M₂-antagonist), and
(b) a muscarinic receptor antagonist selected from the group consisting of non-selective, peripheral anticholinergic agents (nsPAChAs); and, optionally,
(c) an AChEI.

This triple combination may be used for the treatment of hypocholinergic disorders as herein above defined.

According to an embodiment, said pharmaceutical combination comprises, as Components:
(a) a M2-antagonist, in a pharmaceutical composition in admixture with a pharmaceutical carrier; and
(b) a nsPAChA, in a pharmaceutical composition in admixture with a pharmaceutical carrier.

According to another embodiment, said pharmaceutical combination comprises, as Components:
(a) a M₂-antagonist, in a pharmaceutical composition in admixture with a pharmaceutical carrier; and
(b) a nsPAChA selected from the group consisting of oxybutynin and pharmaceutically acceptable salts thereof, in a pharmaceutical composition consisting of a TTS, in admixture with a pharmaceutical carrier.

A preferred pharmaceutical combination comprises
(a) a M₂-antagonist selected from the group consisting of alvameline and pharmaceutically acceptable salts thereof, in a pharmaceutical composition in admixture with a pharmaceutical carrier; and
(b) a nsPAChA consisting of oxybutynin and pharmaceutically acceptable salts thereof, in a TTS in admixture with a pharmaceutical carrier, said TTS being a transdermal patch.

In another embodiment, the combination of the present invention may further comprise a component (c), which is an AChEI.

According to a further embodiment, said pharmaceutical combination comprises as Components:
(a/b) a pharmaceutical composition in dosage unit form comprising
   (a) a M₂-antagonist; and
   (b) a nsPAChA,
   in admixture with a pharmaceutical carrier; and
(c) an AChEI, in admixture with a pharmaceutical carrier.

A preferred pharmaceutical combination comprises
(a) a M₂-antagonist selected form the group consisting of alvameline and pharmaceutically acceptable salts thereof, in a pharmaceutical composition in admixture with a pharmaceutical carrier;
(b) a nsPAChA selected from the group consisting of trospium pharmaceutically acceptable salts, glycopyrronium pharmaceutically acceptable salts, propiverine and pharmaceutically acceptable salts, solifenacin and pharmaceutically acceptable salts thereof, and TTS-oxybutynin, in a pharmaceutical composition in admixture with a pharmaceutical carrier; and
(c) an AChEI selected from the group consisting of donepezil and pharmaceutically acceptable salts thereof in admixture with a pharmaceutical carrier in an IR oral formulation, and rivastigmine in admixture with a pharmaceutical carrier in a patch for transdermal administration.

This combination may be used for the treatment of Alzheimer type dementia and more generally for hypocholinergic disorders of the central nervous system as defined herein above.

In a particular embodiment, the present invention provides a method of treating schizophrenia, schizophrenia associated dementia, or schizoaffective disorders comprising administering to a mammalian, preferably human, subject in need thereof, a combination comprising:
(a) a M₂-muscarinic cholinergic receptor antagonist (M₂-antagonist); and
(b) a non-selective peripheral anticholinergic agent (nsPAChA).

In another particular embodiment, the method of treating schizophrenia, schizophrenia associated dementia, or schizoaffective disorders of the present invention may further comprise administering, as a Component (c), an AChEI.

In a particular embodiment, the present invention provides a method of treating AD or AD type dementia comprising administering to a mammalian, preferably human, subject in need thereof, a combination comprising:
(a) a M₂-muscarinic cholinergic receptor antagonist (M₂-antagonist); and
(b) a non-selective peripheral anticholinergic agent (nsPAChA).

In another particular embodiment, the method of treating AD or AD type dementia of the present invention may further comprise administering a Component (c), an AChEI.

The present invention also provides a combination as described herein wherein Components (a) and (b) are formulated in the same unit form. In another embodiment, a further Component (c), an AChEI, may be formulated in the same unit form.

The present invention also provides a combination as described herein as a fixed-dose combination wherein Components (a) and (b) are formulated in the same unit form.

The present invention also provides a combination as described herein as a fixed-dose combination, as a pharmaceutical composition wherein Components (a) and (b) are formulated in the same unit form in admixture with a pharmaceutical carrier or vehicle. Said fixed dose combination may also comprise said optional Component (c).

Thus,
the M₂-antagonist Component (a), in admixture with a pharmaceutical carrier or vehicle, may be combined with a fixed-dose Combination (b/c) essentially consisting of a pharmaceutical composition in dosage unit form comprising the nsPAChA Component (b) and the AChEI Component (c), in admixture with a pharmaceutical carrier or vehicle;
the nsPAChA Component (b), in admixture with a pharmaceutical carrier or vehicle, may be combined with a fixed-dose Combination (a/c) essentially consisting of a pharmaceutical composition in dosage unit form comprising the M₂-antagonist Component (a) and the AChEI Component (c), in admixture with a pharmaceutical carrier or vehicle; and
the AChEI Component (c), in admixture with a pharmaceutical carrier or vehicle, may be combined with a fixed-dose Combination (a/b) essentially consisting of a pharmaceutical composition in dosage unit form comprising the M₂-antagonist Component (a) and the nsPAChA Component (b), in admixture with a pharmaceutical carrier or vehicle.

The present invention also provides a kit or package containing a combination as described herein, accompanied by instructions for use. In particular, a kit of the present invention is a kit comprising a combination of medicaments for the treatment of hypocholinergic disorders of the CNS.

According to the present invention, the kit allows for the maximal functional capacity and safety during the treatment of a patient with a combination wherein the components may be administered concurrently or sequentially.

More particularly, the kit of the present invention comprises
(a) a pharmaceutical composition in IR or ER dosage unit form comprising or consisting essentially of a therapeutically effective amount of a M₂-antagonist in admixture with a pharmaceutical carrier; and
(b) a pharmaceutical composition in IR or ER dosage unit form comprising or consisting essentially of a therapeutically effective amount of a nsPAChA in admixture with a pharmaceutical carrier;
   for concurrent, sequential or separate administration.

The kit according to the present invention may also comprise an AChEI Component (c), also in an IR or ER form, in admixture with a pharmaceutical carrier in a composition formulated according to known technologies.

Finally, the present invention provides a kit comprising a combination selected from the group consisting of
a combination comprising (a) a M₂-antagonist in a pharmaceutical composition in a dosage unit form wherein said M₂-antagonist is in admixture with a pharmaceutical carrier; and (b) a nsPAChA in a dosage unit form, wherein said combination is in admixture with a pharmaceutical carrier;
a combination that is a fixed dose combination comprising (a) a M₂-antagonist in a pharmaceutical composition in a dosage unit form wherein said M₂-antagonist is in admixture with a pharmaceutical carrier; and (b) a nsPAChA in a pharmaceutical composition in a dosage unit form wherein said nsPAChA is in admixture with a pharmaceutical carrier;
a combination comprising (a) a M₂-antagonist in a pharmaceutical composition in a dosage unit form wherein said M₂-antagonist is in admixture with a pharmaceutical carrier; and (b/c) a fixed-dose combination comprising a nsPAChA and an AChEI Component (c) in a dosage unit form wherein said combination is in admixture with a pharmaceutical carrier;
a combination comprising (b) a nsPAChA in a pharmaceutical composition in a dosage unit form wherein said nsPAChA is in admixture with a pharmaceutical carrier; and (a/c) a fixed-dose combination comprising a M₂-antagonist and an AChEI in a dosage unit form wherein said combination is in admixture with a pharmaceutical carrier;
a combination comprising (c) an AChEI in a pharmaceutical composition in a dosage unit form wherein said AChEI is in admixture with a pharmaceutical carrier; and (a/b) a fixed dose combination comprising a M₂-antagonist and a nsPAChA in a dosage unit form wherein said combination is in admixture with a pharmaceutical carrier; and
a combination comprising (a) a M₂-antagonist in a pharmaceutical composition in a dosage unit form wherein said M₂-antagonist is in admixture with a pharmaceutical carrier; (b) a nsPAChA in a pharmaceutical composition in a dosage unit form wherein said nsPAChA is in admixture with a pharmaceutical carrier; and (c) an AChEI in a pharmaceutical composition in a dosage unit form wherein said AChEI is in admixture with a pharmaceutical carrier.

The kit, which may also contain an AChEI as described herein, can simplify the administration of the combination of the present invention to patients suffering from hypocholinergic disorders of the CNS, who are often not sufficiently able to manage multiple packages.

The finding of the present invention represents surprising and unexpected progress in the treatment of hypocholinergic disorders, especially in view of the lack of efficacy of M₂-antagonists at doses previously administered to human beings, and of the intolerable adverse effects induced by said antagonists at the administered doses.

It has been found that a nsPAChA, when concurrently or sequentially administered in combination with a M₂ muscarinic antagonist allows for the safe administration of said M₂ antagonist, at high doses or doses at or above a maximally tolerated dose of the M₂-antagonist administered alone, thus, in case of a patient suffering from a hypocholinergic disorder such as AD type dementia, schizophrenia, schizophrenia associated dementia, or schizoaffective disorders, allowing said M₂ muscarinic antagonist to more safely activate brain cholinergic receptors and to better improve the cognitive response or increase the therapeutic efficacy of said M₂ muscarinic antagonist for treating the hypocholinergic disorder.

According to literature publications, M₂ antagonists, while relatively promising in animal models, were poorly tolerated in humans and long abandoned. In the sole case of a trial for evaluating the possibility of improving the cognition of patients with a probable Alzheimer disease, the involved product was inefficacious and might even have worsened the cognition in said patients. In addition, because M₂ muscarinic receptor antagonists caused intolerable adverse effects in the tolerability tests in humans, further studies of these products were discouraged.

On the contrary, the present inventors found that the administration of a M₂ muscarinic receptor antagonist concurrently with a nsPAChA substantially attenuates dose-limiting adverse effect not only at the M₂ muscarinic antagonist doses that have been administered to a human, but also at higher doses which were intolerable for said human.

Surprisingly, this combination provides successful treatment of hypocholinergic disorders as defined herein above, such as Alzheimer type dementias, schizophrenia, schizophrenia associated dementia, and schizoaffective disorders, thus allowing said treatment at doses that previously produced dose limiting adverse reactions and even allowing treatment at doses higher than those that caused intolerable dose-limiting adverse events. Without being bound by theory, it is believed that the dose-limiting adverse reactions are due exclusively, or largely exclusively, to over-stimulation of peripheral cholinergic receptors of the muscarinic type. This is particularly true of the M₂-antagonists, and is a major reason why the present inventors believe development of such drugs has long stagnated. That is, the present inventors believe that attempts to clinically exploit the potential cognitive benefits of central M₂ blockade have been inhibited by the simultaneous ability of M₂-antagonists to act outside the CNS to block M₂ receptors. As a result, cholinergic activation produced dose-limiting side effects that precluded administration of effective dosages. The development of M₂ muscarinic antagonists languished because the preclinical results and especially the clinical results were disappointing, not due to a basic lack of muscarinic activity, but because said drugs were inefficacious in patients at doses that did not induce dose-limiting, intolerable adverse effects. Thus, it has been found that the efficacy of M₂ antagonists can be achieved for treatment of hypocholinergic disorders, such as dementias of the Alzheimer type, in a patient suffering from said disorder, by administering a combination of a M₂ antagonist and a nsPAChA as described herein.

### The M₂-Antagonists Component (a)

Any M₂-antagonist which is able to cross the brain blood barrier of a human in order to block the presynaptic muscarinic M₂-receptor thus allowing the increase of acetylcholine transmission in the CNS may be used as Component (a) according to the present invention.

The M₂-antagonists used as Component (a) are muscarinic receptor antagonists that are selective—as herein above defined—for the M₂-receptor subtype.

Preferred Component (a) is a M₂-antagonist selected from the group consisting of
5-(2-ethyl-2H-tetrazol-5-yl)-1-methyl-1,2,3,6-tetrahydropyridine (alvameline) and pharmaceutically acceptable salts and solvates thereof, which can be prepared for example as described in USRE 36374E, the disclosure of which is incorporated herein in its entirety by reference, and used as free base or as its tartrate salt;
5,11-dihydro-8-chloro-11-[[4-[3-[(2,2-dimethyl-1-oxopentyl)ethylamino]propyl]-1-piperidinyl]acetyl]-6H-pyrido[2,3-b][1,4]benzodiazepin-6-one (BIBN-99) and pharmaceutically acceptable salts and solvates thereof, which can be prepared for example as described in U.S. Pat. No. 5,641,772, the disclosure of which is incorporated herein in its entirety by reference, and used as free base or as its dihydrochloride salt;
(±)11-[[2-[(diethylamino)methyl]-1-piperidinyl]acetyl]-5,11-dihydro-6H-pyrido[2,3-b][1,4]benzodiazepin-6-one (otenzepad—AF-DX 116) and pharmaceutically acceptable salts and solvates thereof, which can be prepared, in particular as the monomethanesulfonate salt thereof, for example as described in U.S. Pat. No. 4,550,107, the disclosure of which is incorporated herein in its entirety by reference, and used as free base or as its maleate (1:1), fumarate (1:1), dihydrochloride, dihydrobromide or monomethanesulfonate salt;
(+)11-[[2-[(diethylamino)methyl]-1-piperidinyl]acetyl]-5,11-dihydro-6H-pyrido[2,3-b][1,4]benzodiazepin-6-one [(+)-otenzepad, AF-DX 250] and pharmaceutically acceptable salts and solvates thereof, which can be prepared for example as described in U.S. Pat. No. 4,550,107, the disclosure of which is incorporated herein in its entirety by reference, or as illustrated in J. Med. Chem. 32(8), 1718-24, 1989 (Engel et al. 1989), the disclosure of which is incorporated herein in its entirety by reference, and used as free base or as its dihydrobromide salt;
N-(2-[(2R)-2-[(dipropylamino)methyl]piperidin-1-yl]ethyl)-6-oxo-5H-pyrido[2,3-b][1,4]benzodiazepine-11-carboxamide (AF-DX 384) and pharmaceutically acceptable salts and solvates thereof, which can be prepared for example as described in U.S. Pat. No. 4,873,236, the disclosure of which is incorporated herein in its entirety by reference, and used as free base or as its sesquimaleate, hydrochloride, dihydrochloride dihydrate, hydrobromide, sulfate or methanesulfonate salt;
11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof, which can be prepared for example as described in U.S. Pat. No. 5,175,158, the disclosure of which is incorporated herein in its entirety by reference;
6-[(2-methoxyphenyl)methyl-methylamino]-N-[8-[6-[(2-methoxyphenyl)methyl-methylamino]hexanoyl-methylamino]octyl]-N-methylhexanamide (caproctamine) and pharmaceutically acceptable salts and solvates thereof;
N,N″-(dithiodi-2,1-ethanediyl)bis[N′-[(2-methoxyphenyl)methyl]-1,6-hexanediamine] (benextramine) and pharmaceutically acceptable salts and solvates thereof;
N,N′-bis[6-[(2-methoxybenzyl)amino]hexyl]-1,8-octanediamine (methoctramine) and pharmaceutically acceptable salts and solvates thereof, which can be prepared for example as described in Chemistry and Industry (London) 89(19), 652-653 (Minarini 1989); and in J. Med. Chem 87 Volume 30 (1), 201-204 (Melchiorre 1987), and used as free base or as its hydrochloride or tetrahydrochloride hemihydrate salt;
4-cyclohexyl-α-[4[[4-methoxyphenyl]sulphinyl]-phenyl]-1-piperazineacetonitrile (SCH-57790) and pharmaceutically acceptable salts and solvates thereof, which can be prepared for example as described in the aforementioned Lowe et al. WO 96/26196), the disclosure of which is incorporated herein in their entirety by reference;
4-(4-(1(S)-(4-((1,3-benzodioxol-5-yl)sulfonyl)phenyl)ethyl)-3(R)-methyl-1-piperazinyl)-4-methyl-1-(propylsulfonyl)piperidine (SCH-72788) and pharmaceutically acceptable salts and solvates thereof, also obtainable as described in WO 96/26196;
1′-(2-methylbenzoyl)-4-[[[(3,4-methylenedioxyphenyl)sulfonyl]phenyl]methyl]-1,4′-bipiperidine (SCH-76050) and pharmaceutically acceptable salts and solvates thereof, also obtainable as described in WO 96/26196;
1′-(2-amino-3-methylbenzoyl)-4-[[[(3-chlorophenyl)sulfonyl]phenyl]methyl]-1,4′-bipiperidine (SCH-211803) and pharmaceutically acceptable salts and solvates thereof, which can be prepared for example as described in U.S. Pat. No. 6,294,554, the disclosure of which is incorporated herein in its entirety by reference, and used as free base or as its hydrochloride;
1′-(2-methylbenzoyl)-4-[[[(3,4-methylenedioxyphenyl)sulfonyl]phenyl]methyl]-1,4′-bipiperidine (SCH-76050) and pharmaceutically acceptable salts and solvates thereof, also obtainable as described in WO 96/26196;
1′-(2-amino-3-methylbenzoyl)-4-[[[(3-chlorophenyl) sulfonyl]phenyl]ethylenedioxy methyl]-1,4′-bipiperidine (SCH-217443) and pharmaceutically acceptable salts and solvates thereof which can be prepared for example as described in U.S. Pat. No. 6,294,554, the disclosure of which is incorporated herein in its entirety by reference, and used as free base or as its hydrochloride;
1′-naphto-1-yl-4-[[4-[(methoxycarbonyl)methylthio]phenyl]methyl]-1,4′-bipiperidine (Wang Compound 30); and pharmaceutically acceptable salts and solvates thereof,
1′-(indol-4-yl)carbonyl-4-[[(4-isopropyl)carbonyl]phenyl]methyl]-1,4′-biperidine and pharmaceutically acceptable salts and solvates thereof, (Palani Compound 19);
1′-(indol-4-yl)carbonyl-4-[[(4-isopropyl)carbonyl]phenyl]ethylenedioxymethyl]-1,4′-biperidine and pharmaceutically acceptable salts and solvates thereof, (Palani Compound 30);
N,N-dimethyl-3-[1-(2-pyridinyl)ethyl]-1H-indene-2-ethanamine (dimethindene) and pharmaceutically acceptable salts and solvates thereof, in particular its maleate;
N,N-dimethyl-3-[(1S)-1-(2-pyridinyl)ethyl]-1H-indene-2-ethanamine [(S)-(+)-dimethindene] and pharmaceutically acceptable salts and solvates thereof;
10,19-dimethyl-1,28-bis(6-oxo-5,6-dihydro-11H-pyrido[2,3-b][1,4]benzodiazepin-11-yl)-3-[2-oxo-2-(6-oxo-5,6-dihydro-11H-pyrido [2,3-b][1,4]benzodiazepin-11-yl)ethyl]-3,10,19,26-tetraazaoctacosane-1,28-dione (tripitramine) and pharmaceutically acceptable salts and solvates thereof, which can be prepared for example as described in J. Med. Chem. 1993: 36, 3734-3737 (Melchiorre 1993), the disclosure of which is incorporated herein in its entirety by reference, and used as free base or as its sesquifumarate or tetraoxalate salts;
(3aR,4R,4aS,8aR,9aS)-4-{(E)-[(2R,6S)-1,6-dimethylpiperidin-2-yl]vinyl}-3-methyldecahydronaphtho [2,3-c]furan-1 (3H)-one (himbacine);
(+)-Himbacine, which can be prepared as described in J Org Chem. 1999, Mar. 19; 64(6):1932-1940 (Chackalamannil 1999), and pharmaceutically acceptable salts and solvates thereof;
(3aR,4R,4aS,8aR,9aS)-4-{(E)-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof.

For use for the treatment of hypocholinergic disorders as herein above defined, the present invention provides a pharmaceutical combination comprising:
(a) a muscarinic receptor antagonist selected from the group consisting of M₂-muscarinic cholinergic receptor antagonists (M₂-antagonist); and
(b) a muscarinic receptor antagonist selected from the group consisting of the non-selective, peripheral anticholinergic agents (nsPAChAs).

The M₂-antagonists are formulated in a pharmaceutical composition in IR-form or in ER-form, including a TTS, in admixture with a pharmaceutical carrier or vehicle. The compositions in dosage unit form contain said M₂-antagonist at a dose which is dependent on the intrinsic potency of said M₂-antagonist and is from 0.5 mg to 1500 mg, normally from 0.5 mg to 1000 mg, when included in a pharmaceutical composition in dosage unit form, as set forth above, wherein said M₂-antagonist is present alone or in combination with a nsPAChA in a fixed-dose combination in said unit form.

For their use in combination with a nsPAChA for the treatment of Alzheimer type dementias, schizophrenia, schizophrenia associated dementia, schizoaffective disorders and other hypocholinergic disorders described herein, said M₂-antagonists are administered to patients in need of said treatment at a daily dose of from 1.5 mg to 3000 mg, or from 1.5 mg to 1500 mg by any administration route.

The amount of the M₂-antagonist Component (a) of the combination, i.e. a single M₂-antagonist dose, may vary according to intrinsic muscarinic cholinergic receptor potency of said component, in particular in the aforementioned range of from 0.5 mg to 1500 mg per dosage unit form.

The pharmaceutical composition comprising the M₂-antagonist is used, in combination with a nsPAChA Component (b), which may be even further combined with an AChEI Component (c); for the treatment of a patient suffering from any of the hypocholinergic disorders described herein with a dose of M₂-antagonist heretofore never tested for the adverse effects exhaustively illustrated herein above and in the literature.

The compositions are preferably formulated in dosage unit forms for oral, including buccal (as orodispersible or orosoluble preparations), topical, transmucosal or parenteral, in particular, transdermal, administration, wherein the active ingredient is mixed with a pharmaceutical carrier.

Typically, in said compositions,
alvameline, as free base or a salt or solvate thereof, especially as its tartrate, may be present in an amount, in alvameline, of from 160 mg to 960 mg, preferably from 240 mg to 960 mg;
tripitramine, as free base or a salt or solvate thereof, especially as its sesquifumarate or tetraoxalate salt, may be present in an amount of from 10 mg to 200 mg, preferably from 25 mg to 100 mg;
dimethindene, preferably as the maleate thereof, is present as racemate or as its S(+) enantiomer, in an amount of from 1.1 mg to 32 mg, preferably from 1.2 mg to 15 mg, from 1.5 mg to 8 mg, from 1.5 to 6 mg or from 1.5 to 4 mg in a IR-form or, as the free base or the maleate thereof, in an amount of from 3 mg to 32 mg, preferably from 4 mg to 32 mg, from 4.4 to 32 mg, from 6 mg to 32 mg, from 6 mg to 16 mg or from 3 mg to 10 mg, in an ER-form, including a TTS;
otenzepad, as free base or as the maleate (1:1), fumarate (1:1), dihydrochloride, dihydrochloride dihydrate, dihydrobromide or the monomethanesulfonate (INN: monomesilate, USAN: monomesylate) thereof, in an amount of from 100 mg to 500 mg, preferably from 150 mg to 350 mg in a IR unit form or as the free base or as one of the aforementioned salts, in an amount of from 200 mg to 500 mg, preferably from 300 mg 500 mg, in an ER-form, including a TTS;
AQ-RX 741, as free base or as a salt or solvate thereof, especially as its monomethanesulfonate salt, may be present in an amount of from 10 mg to 500 mg, preferably from 10 mg to 250 mg in a IR-form or, as the free base or the methanesulfonate salt thereof, in an amount of from 20 mg to 500 mg, preferably from 50 mg to 500 mg, in an ER-form, including a TTS.

These compositions are destined for their use in the treatment of hypocholinergic disorders as defined herein above by administering said compositions once, twice or three times per day.

According to an advantageous embodiment, the pharmaceutical compositions prepared by using the M₂-antagonist Component (a), which acts as a cholinergic agent in the CNS to improve the symptoms of Alzheimer type dementia, in a quantity sufficient to maximally alleviate disease-associated neurobehavioral symptoms, are present in unit forms also containing other active ingredients, in particular the nsPAChA Component (b) to form a fixed-dose combination assuring a minimum of treatment-associated adverse effect according to the present invention, said fixed dose combination may be further concurrently or sequentially administered in combination with a pharmaceutical composition in dosage unit form comprising an AChEI in admixture with a pharmaceutical carrier.

### The nsPAChAs Component (b)

Any nsPAChAs, exhibiting inhibitory activity broadly across the various subtypes of muscarinic M-receptors, namely the M₁-M₅ receptors, as currently identified and are largely unable (have a limited ability) to enter the central nervous system following systemic administration and thus do not affect brain function to a clinically appreciable degree may be used as Component (b) according to the present invention. These nsPAChAs include quaternary ammonium salts, sulfonium salts and tertiary amine anticholinergic agents, especially those having low lipid solubility.

The 4-diethylaminobut-2-ynyl 2-cyclohexyl-2-hydroxy-2-phenylethanoate, known under its International Non-proprietary Name as oxybutynin, as free base or a pharmaceutically acceptable salt thereof, is a well-known non-selective anticholinergic medication used by oral route to relieve urinary and bladder difficulties, including frequent urination and urge incontinence and all the above references emphasize this use. Thus, oxybutynin is a very good tool for administering anticholinergic therapy, but it is not “peripheral” as per the definition given above because it is able to cross the blood brain barrier (“BBB”) to a non-negligible extent (Rebecca J McCrery and Rodney A Appell, Ther Clin Risk Manag. March 2006; 2/1: 19-24).

The literature discloses pharmaceutical compositions and Transdermal Therapeutic Systems (TTS) delivering oxybutynin through the human skin.

For example, U.S. Pat. No. 5,411,740 and U.S. Pat. No. 5,500,222, the disclosures of which are herein incorporated by reference in their entirety, disclose a patch for the transdermal administration of oxybutynin base using a monoglyceride or a mixture of monoglycerides of fatty acids as skin permeation-enhancer.

U.S. Pat. No. 5,686,097; U.S. Pat. No. 5,747,065; U.S. Pat. No. 5,750,137 and U.S. Pat. No. 5,900,250, the disclosures of which are herein incorporated by reference in their entirety, disclose a patch for the transdermal administration of oxybutynin base using a monoglyceride or a mixture of monoglycerides plus a lactate ester as skin permeation-enhancer.

A similar patch, adding a non-rate controlling tie layer on the skin-proximal surface of the reservoir, not affecting the drug release, is described in U.S. Pat. No. 5,614,211 and U.S. Pat. No. 5,635,203, the disclosures of which are herein incorporated by reference in their entirety.

U.S. Pat. No. 5,212,199, U.S. Pat. No. 5,227,169, U.S. Pat. No. 5,601,839 and U.S. Pat. No. 5,834,010, the disclosures of which are incorporated herein by reference in their entirety, disclose a patch for transdermal administration of basic drugs using triacetin as permeation enhancer.

U.S. Pat. No. 6,555,129, the disclosure of which is herein incorporated by reference in its entirety, discloses a TTS substantially consisting of an oxybutynin-containing matrix mass in the form of a layer which is self-adhesive, and in which the matrix mass consists of ammonium-group-containing (meth)acrylate copolymers, at least one citric acid triester and 5-25% by weight of oxybutynin.

U.S. Pat. No. 6,562,368, the disclosure of which is herein incorporated by reference in its entirety, discloses a method for transdermally administering oxybutynin using a composition in form of a patch, a cream, a gel, a lotion or a paste comprising oxybutynin and a hydroxide-releasing agent substantially consisting of inorganic hydroxides, inorganic oxides, metal salts of weak acids, and mixtures thereof.

U.S. Pat. Nos. 6,743,411; 7,081,249; U.S. Pat. No. 7,081,250; U.S. Pat. No. 7,081,251; U.S. Pat. No. 7,081,252 and U.S. Pat. No. 7,087,241, the disclosures of which are herein incorporated by reference in their entirety, disclose a transdermal patch delivering a composition comprising oxybutynin to a subject to provide a plasma area under the curve ratio of oxybutynin to an oxybutynin metabolite of from about 0.5:1 to about 5:1, optional in the presence of a permeation enhancer.

U.S. Pat. No. 7,029,694; U.S. Pat. No. 7,179,483; U.S. Pat. No. 8,241,662 and US 2009/0018190, the disclosures of which are herein incorporated by reference in their entirety, disclose a transdermal gel formulation comprising oxybutynin providing a plasma area under the curve ratio of oxybutynin to an oxybutynin metabolite of from about 0.5:1 to about 5:1, optional in the presence of a permeation enhancer.

US 2004/0219194, the disclosure of which is herein incorporated by reference in its entirety, discloses a transdermal therapeutic system containing oxybutynin, triacetin and Aloe vera extract as permeation enhancer.

US 2004/0057985, the disclosure of which is herein incorporated by reference in its entirety, discloses transdermal therapeutic systems (TTS) for the administration of oxybutynin with which therapeutically active absorption rates can be achieved without the necessity of adding permeation-enhancing substances. These TTS comprise a substantially water vapor-impermeable backing layer, at least one pressure-sensitive adhesive matrix layer attached thereto, and a detachable protective film, said matrix layer comprising an inner phase containing the active substance oxybutynin, and an outer, pressure sensitive adhesive phase based on hydrocarbon polymers or/and silicone polymers.

US 2005/0064037, the disclosure of which is herein incorporated by reference in its entirety, discloses an oxybutynin topical gel formulation comprising oxybutynin chloride salt, a short chain alcohol, a gelling agent substantially consisting of high-molecular-weight, cross-linked polymer of acrylic acid or cross-linked copolymer of acrylic acid and C10-30 alkyl acrylate, and optionally a permeation enhancer substantially consisting of propylene glycol, propylene glycol laurate, isopropyl myristate, and methyl lactate.

WO 2005/039531, US2007/022379, US 2010/0216880, US 2014/0037713 and U.S. Pat. No. 8,652,491, the disclosures of which are herein incorporated by reference in their entirety, disclose a transdermal or transmucosal pharmaceutical formulation, that can be utilized for topical or transdermal application, such as in solutions, creams, lotions, sprays, ointment, gels, aerosols and patch devices, for the delivery of one or more active agents, including anticholinergics, in particular oxybutynin. Said formulation includes oxybutynin in a solvent system comprising a diethylene glycol monoalkyl ether and a glycol in specific ratios, alcohol and water. In particular, according to U.S. Pat. No. 8,652,491 a possible secondary active agent, in addition to the anticholinergic agent such as oxybutynin, may be an antiperspirant, a tranquilizer or another agent capable of ameliorating hyperhidrosis. In addition, according to WO 2005/039531 the active agent may also be selected from an anti-Alzheimer's drug, in particular galantamine, rivastigmine, donepezil, tacrine, or memantine, without giving any indication of the doses to be used.

WO 2005/107812, U.S. Pat. No. 7,425,340 and US 2008/0260842, the disclosures of which are herein incorporated by reference in their entirety, disclose formulations containing an anticholinergic agent, in particular oxybutynin, in admixture with urea, urea congeners or urea-containing compounds as permeation enhancers.

WO 01/07018 and U.S. Pat. No. 8,420,117, the disclosures of which are herein incorporated by reference in their entirety, disclose a matrix patch formulation containing no water for external use, comprising, as essential components oxybutynin hydrochloride, citric acid and sodium acetate.

WO2013/061969 and US 2014/0271796, the disclosures of which are herein incorporated by reference in their entirety, disclose a transdermal absorption preparation comprising at least one drug selected from oxybutynin and pharmaceutically acceptable salts thereof; and a sterol such as cholesterol, cholesterol derivatives and cholesterol analogs.

U.S. Pat. No. 8,802,134, the disclosure of which is herein incorporated by reference in its entirety, discloses a method for producing a patch wherein oxybutynin is incorporated in an adhesive agent layer composition comprises the acrylic-based polymer as the adhesive base agent, and the acrylic-based polymer is a copolymer of polymethyl methacrylate with a polyacrylate.

U.S. Pat. No. 8,877,235, the disclosure of which is herein incorporated by reference in its entirety, discloses a patch consisting of a support layer and of an adhesive agent layer arranged on the at least one surface of the support layer, the adhesive agent layer comprising oxybutynin hydrochloride in a supersaturated concentration in a dissolved form. Said layer also comprises acrylic-based polymers and rubber-based polymers, as adhesive base agents, and liquid paraffin, a sterol, an organic acid, and a tackifier.

Oxybutynin is also commercially presented in a 39-cm² patch system containing 36 mg of oxybutynin and releasing 3.9 mg/day oxybutynin (OXYTROL®). This patch provides significant improvements in all the measured parameters with less systemic adverse effects, as summarized by J. Jayarajan and S. B. Radomski in a review presented on 4 Dec. 2013: “Pharmacotherapy of overactive bladder in adults: a review of efficacy, tolerability, and quality of life” (J. Jayarajan et al., Research and Reports in Urology 2014:6), the disclosure of which is herein incorporated by reference in its entirety. However, oxybutynin is anyway deemed to cross the BBB owing to its high lipophilicity, neutrality, and small molecular size (C. A. Donnellan et al. BMJ 1997; 315:1363-4; R. Scheife and M. Takeda, Clin Ther. 2005; 27:144-53). the disclosure of which is herein incorporated by reference in its entirety.

Even when given by transdermal route, oxybutynin has been shown to penetrate the brain. Studies with radiolabeled [¹⁴C] oxybutynin administered transdermally to rats have shown presence of radiolabel in the brain [Pharmaceutical and Medical Devices Agency Interview Form (PMDA is the Japanese Regultaory Agency, equivalent to FDA in the US)].

Oxybutynin is also commercially presented in a 39-cm² patch system containing 36 mg of oxybutynin and releasing 3.9 mg/day oxybutynin (OXYTROL®). This patch provides significant improvements in all the measured parameters with less systemic adverse effects, as summarized by J. Jayarajan and S. B. Radomski in a review presented on 4 Dec. 2013: “Pharmacotherapy of overactive bladder in adults: a review of efficacy, tolerability, and quality of life” (J. Jayarajan et al., Research and Reports in Urology 2014:6), the disclosure of which is herein incorporated by reference in its entirety. However, oxybutynin is anyway deemed to cross the BBB owing to its high lipophilicity, neutrality, and small molecular size (C. A. Donnellan et al. BMJ 1997; 315:1363-4; R. Scheife and M. Takeda, ClinTher. 2005; 27:144-53). the disclosure of which is herein incorporated by reference in its entirety.

Even when given by transdermal route, oxybutynin has been shown to penetrate the brain. Studies with radiolabeled [¹⁴C] oxybutynin administered transdermally to rats have shown presence of radiolabel in the brain [Pharmaceutical and Medical Devices Agency Interview Form (PMDA is the Japanese Regulatory Agency, equivalent to FDA in the US)].

Oxybutynin is also commercially presented (GELNIQUE®) in a TTS consisting of a hydroalcoholic gel containing 100 mg oxybutynin chloride per gram of gel and available in a 1 gram (1.14 ml) unit dose. This TTS is deemed to have a pharmacokinetic profile similar to that of the patch delivery system, while producing lower N-desethyloxybutynin metabolite plasma concentrations (Vincent R Lucente et al.; Open Access Journal of Urology 2011/3, 35-42). Another commercial TTS system, presents oxybutynin in a hydroalcoholic gel containing 30 mg oxybutynin base per gram of gel and is available (ANTUROL®) in a 0.92 gram (1 mL) unit dose that contains 28 mg oxybutynin per gram of gel. Also Anturol® demonstrated plasma levels of oxybutynin comparable to the efficacious plasma levels observed for oral and patch therapies with lower N-desethyloxybutynin plasma levels (Anturol® Gel Summary by Antares Pharma).

The label for transdermal oxybutynin warns that a variety of CNS anticholinergic effects have been reported, including headache, dizziness, and somnolence. Patients should be monitored for signs of anticholinergic CNS effects, particularly after beginning treatment. The label further advises that patients should be told not to drive or operate heavy machinery until they know how transdermal oxybutynin affects them. The label also advises that if a patient experiences anticholinergic CNS effects, drug discontinuation should be considered. In addition, the label states that overdosage with oxybutynin has been associated with CNS anticholinergic effects including excitation, memory loss, stupor, disorientation and agitation on awakening. Hence, based on the existing literature, and the competing action of oxybutynin and an AChEI in the CNS, the combined use of such drugs would have made memory loss a-priori material risk for the treatment of Alzheimer type dementia.

The copending applications U.S. Ser. No. 14/991,273 and PCT/US2016/012610 (WO2016/112263) disclose the combination of oxybutynin or a pharmaceutically acceptable salt thereof, in a transdermal therapeutic system (“TTS-oxybutynin”), with an acetylcholinesterase inhibitor for the treatment of Alzheimer type dementia.

According to these documents, it is possible to administer high doses of an AChEI such as rivastigmine, in combination with TTS-oxybutynin without inducing AChEI-associated dose-limiting adverse effects due to the concurrent presence of oxybutynin in the combination. In addition, according to these documents, the treated subjects did not show any sign of central anticholinergic adverse effects such as mental or mood changes (e.g., confusion or memory loss, somnolence or convulsions).

Thus, according to the present invention, contrary to oral oxybutynin and to the TTS-oxybutynin label statement, TTS-oxybutynin may be considered, in every aspect, as a nsPAChA.

The nsPAChAs used as Component (b) may include, but are not limited to, quaternary ammonium nsPAChAs, sulfonium nsPAChAs, (1S)-(3R)-1-azabicyclo[2.2.2]oct-3-yl 3,4-dihydro-1-phenyl-2(1H)-iso-quinolinecarboxylate (solifenacin) and pharmaceutically acceptable salts and solvates thereof, 1-methylpiperidin-4-yl) 2,2-di(phenyl)-2-propoxyacetate (propiverine) and pharmaceutically acceptable salts and solvates thereof, 1,4,5,6-tetrahydro-1-methylpyrimidin-2-ylmethyl α-cyclohexyl-α-hydroxy-α-phenylacetate (oxyphencyclimine) and pharmaceutically acceptable salts and solvates thereof, (R)—N,N-diisopropyl-3-(2-hydroxy-5-methylphenyl)-3-phenylpropanamine (tolterodine) and pharmaceutically acceptable salts and solvates thereof, [2-[(1R)-3-(di(propan-2-yl)amino)-1-phenylpropyl]-4-(hydroxymethyl)phenyl] 2-methylpropanoate (fesoterodine) and pharmaceutically acceptable salts and solvates thereof and 4-diethylaminobut-2-ynyl 2-cyclohexyl-2-hydroxy-2-phenylethanoate (oxybutynin) in a TTS (“TTS-Oxybutynin”).

Said nsPAChAs, preferably, are compounds with a duration of action of at least 6 hours, advantageously from 8 to 24 hours, more advantageously from 10 to 24 hours, preferably from 12 to 24 hours, even though nsPAChAs having an appropriate duration of action corresponding to the duration of action of the concomitantly administered M2-antagonist may be successfully used.

Typical quaternary ammonium nsPAChAs or sulfonium nsPAChAs are compounds of formula I

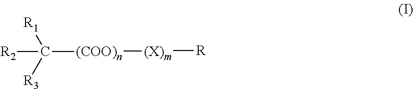

wherein
R is a radical selected from the group consisting of those of formulas (a)-(e)

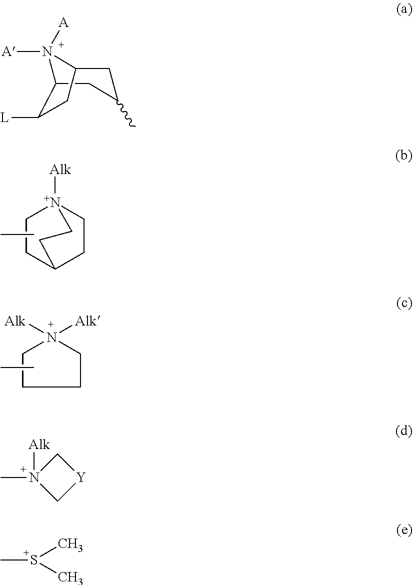

A being methyl and A′ being (C₁-C₄)alkyl or 2-fluoroethyl group or A and A′ forming a 1,4-butylene or 1,5-pentylene chain, L being hydrogen or methoxy, Alk and Alk′ each being (C₁-C₄)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion, such as a chloro, bromo, iodo, tartrate, hydrogen tartrate, succinate, maleate, fumarate, sulfate, hydrogen sulfate or methylsulfate anion;
n and m, independently, are zero or 1;
X is a (C₂-C₃)alkylene group;
R₁ and R₂ are each phenyl, cyclopentyl, cyclohexyl, 1-cyclohexenyl, 2-thienyl and, when R is a radical (a), also each represents (C₁-C₄)alkyl;
R₃ is H or OH or, only when R is a radical (a), also a COOAlk group, Alk being a (C₁-C₄)alkyl group.

Preferably, in the above formula I, at least one of m and n is 1.

Exemplary nsPAChAs of formula I above useful for the treatment of Alzheimer type dementia in combination with M₂-antagonists are
anisotropine methylbromide [R=(a), A=A′=CH₃, L=H; n=1; m=0; R₁=R₂=n-C₃H7; R₃=H;];
ciclotropium bromide [R=(a), A=CH₃, A′=isopropyl, L=H; n=1; m=0; R₁=phenyl; R₂=cyclopentyl; R₃=H];
flutropium bromide [R=(a), A=CH₃, A′=2-fluoroethyl, L=H; n=1; m=0; R₁=R₂=phenyl; R₃=OH];
homatropine methylbromide [R=(a), A=A′=CH₃, L=H; n=1; m=0; R₁=phenyl; R₂=R₃=H];
sintropium bromide; [R=(a), A=CH₃, A′=isopropyl, L=H; n=1; m=0; R₁=R₂=n-C₃H₇; R₃=H];
tematropium metilsulfate [R=(a), A=A′=CH₃, L=H; n=1; m=0; R₁=phenyl; R₂=COOC₂H₅; R₃=H];
tropenziline bromide [R=(a), A=A′=CH₃, L=methoxy; n=1; m=0; R₁=R₂=phenyl, R₃=OH];
trospium chloride [R=(a), A+A′=1,4-butylene, L=H; n=l1; m=0; R₁=R₂=phenyl; R₃=OH];
clidinium bromide [R=(b)-3-, Alk=methyl; n=1; m=0; R₁=R₂=phenyl; R₃=OH];
droclidinium bromide [R=(b)-3-, Alk=methyl; n=l1; m=0; R₁=phenyl; R₂=cyclopentyl; R₃=OH];
benzilonium bromide [R=(c)-3-, both Alk and Alk′=ethyl; n=l1; m=0; R₁=R₂=phenyl; R₃=OH];
benzopyrronium bromide [R=(c)-3-, both Alk and Alk′=methyl; n=1; m=0; R₁=R₂=phenyl; R₃=OH];
cyclopyrronium bromide [R=(c)-3-, Alk=methyl and Alk′=ethyl; n=1; m=0; R₁=phenyl; R₂=cyclopentyl; R₃=H];
glycopyrronium bromide (glycopyrrolate) [R=(c)-3-, both Alk and Alk′=methyl; n=l1; m=0; R₁=phenyl; R₂=cyclopentyl; R₃=H];
heteronium bromide [R=(c)-3-, both Alk and Alk′=methyl n=1; m=0; R₁=phenyl; R₂=2-thienyl; R₃=OH];
hexopyrronium bromide [R=(c)-3-, both Alk and Alk′=methyl; n=1; m=0; R₁=phenyl; R₂=cyclohexyl; R₃=H];
oxypyrronium bromide [R=(c)-2-, both Alk and Alk′=methyl; n=1; m=1; X=1,2-ethylene; R₁=phenyl; R₂=cyclohexyl; R₃=OH];
ritropirronium bromide [R=(c)-3-, both Alk and Alk′=methyl; n=1; m=0; R₁=phenyl; R₂=cyclopentyl; R₃=OH];
etipirium iodide [R=(d), Alk=methyl, Y=1,2-ethylene; n=l1; m=1; X=1,2-ethylene; R₁=R₂=phenyl; R₃=OH];
fenclexonium methylsulfate [R=(d), Alk=CH₃, Y=1,3-propylene; n=0; m=1; X=1,2-ethylene; R₁=phenyl; R₂₌₁-cyclohexenyl; R₃=H];
tricyclamol chloride (procyclidine methochloride) [R=(d), Alk=methyl, Y=1,2-ethylene; n=0; m=1; X=1,2-ethylene; R₁=phenyl; R₂=cyclohexyl; R₃=OH];
tiemonium iodide [R=(d), Alk=methyl, Y=2-oxa-1,3-propylene; n=0; m=1; X=1,2-ethylene; R₁=phenyl; R₂₌₂-thienyl; R₃=OH];
hexasonium iodide [R=(e); n=1; m=1; X=1,2-ethylene; R₁=phenyl; R₂=cyclohexyl; R₃=H]; and
oxysonium iodide [R=(e); n=l1; m=1; X=1,2-ethylene; R₁=phenyl; R₂=cyclohexyl; R₃=OH.

Commercial nsPAChAs for anticholinergic therapy and useful as component (b) of the present invention, include but are not limited to, anisotropine hydrobromide, atropine methobromide, atropine methonitrate, benactizine methobromide, cimetropium bromide, clidinium bromide, dibutoline sulfate, diphemanil, diponium bromide, emeprioum bromide fesoterodine fumarate, glycopyrronium bromide, isopropamide iodide, otilonium bromide, oxyphencyclimine hydrochloride, penthienate bromide, pipenzolate bromide, prifinium bromide, propiverine hydrochloride, scopolamine methobromide, scopolamine butylbromide (butylscopolamine bromide), scopolamine methonitrate, solifenacin succinate, timepidium bromide, TTS-oxybutynin and valethamate bromide.

In particular, a nsPAChA of the present invention may be oxybutynin or a pharmaceutically acceptable salt thereof, in a transdermal therapeutic system (TTS). In the TTS, the oxybutynin or pharmaceutically acceptable salt thereof is in a transdermal formulation incorporated into a patch. For example, oxybutynin is commercially presented in a patch releasing 3.9 mg/day oxybutynin (OXYTROL®). The oxybutynin TTS, in certain embodiments, contains oxybutynin or a pharmaceutically acceptable salt thereof in an amount allowing an oxybutynin release of from 3.9 mg/24 h to 5.85 mg/24 h or from 3.9 mg/24 h to 7.8 mg/24 h.

The oxybutynin TTS for use according to the present invention may be in any oxybutynin delivering transdermal pharmaceutical form, such as a patch, a gel, a cream, a spray, an ointment, a lotion or a paste, wherein oxybutynin is present in admixture with the common diluents and permeation enhancers, said pharmaceutical form containing oxybutynin base or a pharmaceutically acceptable salt thereof, such as its hydrochloride, hydrobromide, sulfate, phosphate, mesilate, acetate, maleate, succinate, lactate, citrate, hydrogen tartrate, tartrate, napsilate or embonate.

Advantageous nsPAChAs are the tertiary amine or quaternary ammonium compounds available in drugs for current anticholinergic therapy, in particular anisotropine hydrobromide, available with a maximum dose/unit form of 50 mg; butylscopolamine bromide, with a maximum dose/unit form of 10 mg; cimetropium bromide, with a maximum dose/unit form of 50 mg; clidinium bromide, with a maximum dose/unit form of 2.5 mg; ER fesoterodine fumarate, with a maximum dose/unit form of 8 mg; glycopyrronium bromide, with a maximum dose/unit form of 2 mg; otilonium bromide, with a maximum dose/unit form of 40 mg; prifinium bromide, with a maximum dose/unit form of 30 mg; IR propiverine hydrochloride, with a maximum dose/unit form of 15 mg; ER propiverine hydrochloride, with a maximum dose/unit form of 30 mg; solifenacin succinate, with a maximum dose/unit form of 10 mg; timepidium bromide, with a maximum dose/unit form of 30 mg; IR-tolterodine tartrate, with a maximum dose/unit form of 2 mg; ER-tolterodine tartrate, with a maximum dose/unit form of 4 mg; IR trospium chloride, with a maximum dose/unit form of 20 mg; ER trospium chloride, with a maximum dose/unit form of 60 mg; TTS-oxybutynin, as free base or as its hydrochloride, available as a patch releasing 3.9 mg/24 h oxybutynin, or as a gel in an amount as illustrated above; and valethamate bromide, with a maximum dose/unit form of 10 mg.

Azoniaspiro [3 (-benziloyloxy-(1α,5α)-nortropane-8,1′-pyrrolidine]chloride (formula I, A+A′=1,4-butylene) described in U.S. Pat. No. 3,480,626 (the disclosure of which is incorporated herein in its entirety by reference), known under its International Non-proprietary Name trospium chloride; the tartrate, maleate, fumarate and succinate salts of trospium; solifenacin, described in U.S. Pat. No. 6,017,927 (the disclosure of which is incorporated herein in its entirety by reference), and the compound thereof with succinic acid; propiverine hydrochloride, described in DD 106643 (the disclosure of which is incorporated herein in its entirety by reference), and its quaternary salts methylpropiverinium iodide and methylpropiverinium bromide, described in WO 2014/025569 (the disclosure of which is incorporated herein in its entirety by reference); oxyphencyclimine, described in GB 795758 (the disclosure of which is incorporated herein in its entirety by reference), and the hydrochloride thereof; tolterodine, described in U.S. Pat. No. 5,382,600 (the disclosure of which is incorporated herein in its entirety by reference), and the hydrogen tartrate thereof; fesoterodine, described in U.S. Pat. No. 5,382,600 (the disclosure of which is incorporated herein in its entirety by reference), and the fumarate thereof; and TTS-oxybutynin, are the preferred nsPAChAs. Other pharmaceutical acceptable salts of trospium, in particular those with succinic acid and tartaric acid, are cited in US 2006/0293356, the disclosure of which is incorporated herein in its entirety by reference.

Glycopyrronium bromide; trospium chloride, which is a long-acting nsPAChA whose absorbed amount, even though poor, has an average plasma half-life of about 18 hours; solifenacin succinate, which also has a long half-life; propiverine hydrochloride and the aforementioned quaternary ammonium salts thereof; and TTS-oxybutynin, are particularly preferred.

In the combination of the present invention, the nsPAChA Component (b) is present, generally in a pharmaceutical composition in admixture with a pharmaceutical carrier or vehicle, in an amount of from 50% to 600% of the amount of the said nsPAChA contained as a sole active ingredient in the aforementioned, currently used brand or generic drugs for anticholinergic therapy.

The nsPAChA Component (b) may be formulated in pharmaceutical compositions comprising, as an active ingredient thereof, said nsPAChA in admixture with a pharmaceutical carrier or vehicle.

Said Component (b) is present in an amount that allows the reduction of peripherally mediated adverse effects caused by the administration of M₂-antagonist.

In a preferred embodiment, the amount of a nsPAChA, such as each of the aforementioned tertiary amine and quaternary ammonium nsPAChAs that is commercially available for the anticholinergic therapy, generally is from 0.5 to 6 times or 1.2 to 6-times the maximum amount contained in the IR-forms of the marketed drugs. Advantageously, the nsPAChA amount in a compositions as IR-formulation is from 0.5 to 4 times, preferably from 1.2 to 4 times the maximum amount contained in the commercial drugs in IR form and the nsPAChA amount in a compositions as ER-formulation is from 0.75- to 6-times, preferably from 1.2- to 6-times the maximum amount contained in the marketed drugs in IR form or in an amount of from 0.75-times to 4-times, preferably from 1.2-times to 4-times the maximum amount contained in the marketed drugs in ER form.

Thus, according to this preferred embodiment, the combination of the present invention comprises, as Component (b), a nsPAChA selected from the group consisting of anisotropine methylbromide, in an amount from 25 mg to 300 mg, advantageously from 60 mg to 300 mg, normally from 60 mg to 200 mg; cimetropium bromide, in an amount from 25 mg to 300 mg, advantageously from 60 mg to 300 mg, normally from 60 mg to 200 mg; clidinium bromide, in an amount from 1.25 mg to 30 mg, advantageously from 6 mg to 30 mg, normally from 6 mg to 20 mg; fesoterodine fumarate, in an amount from 4 mg to 48 mg, advantageously from 9.6 mg to 48 mg, normally from 9.6 mg to 32 mg; glycopyrronium bromide, in an amount from 1 mg to 16 mg, advantageously from 2.4 mg to 12 mg, normally from 2.4 mg to 8 mg; otilonium bromide, in an amount from 20 mg to 240 mg, advantageously from 48 mg to 240 mg, normally from 48 mg to 160 mg; oxyphencyclimine hydrochloride, in an amount from 5 mg to 60 mg, advantageously from 12 mg to 60 mg, normally from 12 mg to 40 mg; prifinium bromide, in an amount from 15 mg to 180 mg, advantageously from 36 mg to 180 mg, normally from 36 mg to 120 mg; propiverine hydrochloride, in an amount from 7.5 mg to 180 mg, advantageously from 17.5 mg to 180 mg, normally from 17.5 mg to 120 mg; solifenacin succinate, in an amount from 5 mg to 30 mg, advantageously from 10 mg to 30 mg, normally from 12 mg to 21 mg; timepidium bromide, in an amount from 15 mg to 180 mg, advantageously from 36 mg to 180 mg, normally from 36 mg to 120 mg; trospium chloride, in an amount of from 10 mg to 360 mg, advantageously from 24 mg to 360 mg, normally from 24 mg to 180 mg; TTS-oxybutynin, in a released amount (from a patch) of from 3.9 mg/24 h to 7.8 mg/24 h, advantageously from 3.9 mg/24 h to 5.85 mg/24 h, normally of 3.9 mg/24 h.

Said Component (b) is normally formulated in a pharmaceutical composition in dosage unit form, in admixture with a pharmaceutical carrier or vehicle. For its use in the treatment of hypocholinergic disorders, said Component (b), as a sole active ingredient of the pharmaceutical composition, may be in a commercial preparation.

Advantageously, Component (b) of said combination is a pharmaceutical composition in an IR- or ER-form comprising a nsPAChA selected from the group consisting of anisotropine hydrobromide, in an amount of from 60 mg to 300 mg, in IR or ER form, preferably from 60 mg to 200 mg in IR form; butylscopolamine bromide, in an amount of from 12 mg to 60 mg in IR or ER form, preferably from 12 mg to 40 mg in IR form; cimetropium bromide, in an amount of from 60 mg to 300 mg in IR or ER form, preferably from 60 mg to 200 mg in IR form; clidinium bromide, in an amount of from 3 mg to 15 mg n IR or ER form, preferably from 3 mg to 10 mg in IR form; fesoterodine fumarate ER, in an amount of from 9.6 mg to 32 mg; glycopyrronium bromide, in an amount of from 2.4 mg to 8 mg in IR or ER form, preferably from 2.4 mg to 4 mg in IR form; otilonium bromide, in an amount of from 48 mg to 160 mg in IR or ER form, preferably from 48 mg to 120 mg in IR form; oxyphencyclimine, in an amount of from 18 mg to 60 mg in IR or ER form, preferably from 18 mg to 40 mg in IR form; prifinium bromide, in an amount of from 36 mg to 120 mg in IR or ER form, preferably from 36 mg to 120 mg in IR form; propiverine hydrochloride IR, in an amount of from 18 mg to 60 mg; propiverine hydrochloride ER, in an amount of from 36 mg to 180 mg; solifenacin succinate, in an amount of from 5 mg to 30 mg, normally from 12 mg to 30 mg or from 12 mg to 21 mg; tolterodine hydrogen tartrate, in an amount of from 4.8 mg to 24 mg in IR or ER form, preferably from 4.8 mg to 16 mg in IR form; timepidium bromide, in an amount of from 36 mg to 180 mg IR or ER form, preferably from 36 mg to 120 mg in IR form; trospium chloride IR, in an amount of from 24 mg to 80 mg; trospium chloride ER, in an amount of from 72 mg to 240 mg; and valethamate bromide, in an amount of from 12 mg to 60 mg in IR or ER form, preferably from 12 mg to 40 mg in IR form; in admixture with a pharmaceutical carrier or vehicle.

In the above combination, propiverine hydrochloride is preferably present in an amount of from 18 mg to 90 mg in an IR-formulated composition, in admixture with a pharmaceutical carrier or in an amount of from 36 mg to 180 mg in an ER-formulated composition, in admixture with a pharmaceutical carrier. Similarly, in the above combination; trospium chloride is preferably present in an amount of from 24 mg to 80 mg in an IR-formulated composition, in admixture with a pharmaceutical carrier or in an amount of from 72 mg to 240 mg in an ER-formulated composition, in admixture with a pharmaceutical carrier and TTS-oxybutynin is preferably present in a patch delivering from 3.9 mg/24 h to 7.8 mg/24 h oxybutynin. Solifenacin succinate is preferably present in an amount selected from the group consisting of from 5 mg to 30 mg, from 10 mg to 30 mg, from 12 mg to 30 mg, from 12 mg to 21 mg.

The compositions prepared using the nsPAChAs as Component (b) of the combination according to the present invention allow the administration of heretofore never administered, high and even very high doses of a M₂-antagonist to patients suffering from hypocholinergic disorders such as Alzheimer type dementia, schizophrenia, schizophrenia associated dementia, and schizoaffective disorders, without clinically significant symptoms of peripheral cholinergic system overstimulation.

The compositions are preferably formulated in dosage unit forms for oral, including buccal (as orodispersible or orosoluble preparation), transmucosal, topical or parenteral, in particular transdermal, administration, wherein the active ingredient is mixed with a pharmaceutical carrier or vehicle.

The pharmaceutical compositions prepared using the nsPAChAs Component (b) according to the present invention are indicated in the treatment of hypocholinergic disorders in combination with even high doses of a M₂-antagonist Component (a), concurrently or sequentially administered therewith, in order to improve to a greater extent said symptoms without adverse effects. The pharmaceutical compositions may further include, as Component (c), an AChEI.

Thus, the invention provides compositions and methods for treating hypocholinergic disorders, which comprises administering to a patient in need of said treatment the above-illustrated combination. In such a treatment, Component (a) and Component (b) of the combination may be administered simultaneously or sequentially to said patient, Compound (a) being indifferently administered before or after Compound (b). Compounds (a) and/or (b) may also be administered by the same or a different administration route.

The invention may also include a third component, Component (c), that is an AChEI, also formulated in a pharmaceutical composition.

### The Combinations

The present invention provides the combination of any M₂-antagonist and any nsPAChA as exemplified in the respective sections herein, each formulated in pharmaceutical composition in admixture with a pharmaceutical carrier.

A typical, preferred M₂-antagonint/nsPAChA combination comprises, as Components:
(a) a M₂-antagonist, in a pharmaceutical composition in dosage unit form, in admixture with a pharmaceutical carrier or vehicle; and
(b) a TTS-oxybutynin as a patch releasing from 3.9 mg/24 h to 7.8 mg/24 oxybutynin, wherein oxybutynin is admixture with a pharmaceutical carrier or vehicle.

A particularly preferred M₂-antagonist/nsPAChA combination comprises or essentially consists of
(a) a M₂-antagonist selected from the group consisting of alvameline and pharmaceutically acceptable salts thereof, in an amount, in alvameline, of from 160 mg to 960 mg, preferably from 240 mg to 960 mg, in a pharmaceutical composition in admixture with a pharmaceutical carrier or vehicle; and
(b) a nsPAChA which is a TTS-oxybutynin as a patch releasing from 3.9 mg/24 h to 7.8 mg/24 oxybutynin, wherein oxybutynin is admixture with a pharmaceutical carrier or vehicle.

According to a first embodiment, the combination of the present invention may be a combination comprising or consisting essentially of
(a) any of the M₂-antagonists such as those described herein above, each in a pharmaceutical composition in dosage unit form, in admixture with a pharmaceutical carrier, said M₂-antagonist being preferably selected from the group consisting of 5-(2-ethyl-2H-tetrazol-5-yl)-1-methyl-1,2, 3,6-tetrahydropyridine (alvameline) and pharmaceutically acceptable salts and solvates thereof), 5,11-dihydro-8-chloro-11-[[4-[3-[(2,2-dimethyl-1-oxopentyl)ethylamino]propyl]-1-piperidinyl]acetyl]-6H-pyrido[2,3-b][1,4]benzodiazepin-6-one (BIBN-99) and pharmaceutically acceptable salts and solvates thereof; racemic 11-[[2-(Diethylamino)methyl]-1-piperidinyl]-acetyl]-5,11-dihydro-6H-pyrido[2,3-b][1,4] benzodiazepin-6-one (otenzepad) and pharmaceutically acceptable salts and solvates thereof; dextrorotatory 11-[[2-(diethylamino)methyl]-1-piperidinyl]-acetyl]-5,11-dihydro-6H-pyrido [2,3-b]E1,4] benzodiazepin-6-one [(+)-otenzepad] and pharmaceutically acceptable salts and solvates thereof; N-2-[2-[(dipropylamino)methyl]-1-piperidinyl]ethyl]-5-,6-dihydro-11-H-pyrido[2,3-b][1,4]benzodiazepine-11-carboxamide (AF-DX 384) and pharmaceutically acceptable salts and solvates thereof; 11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof; N,N-Dimethyl-3-[1-(2-pyridinyl)ethyl]-1H-indene-2-ethanamine (dimethindene) and pharmaceutically acceptable salts and solvates thereof; N,N-Dimethyl-3-[(1S)-1-(2-pyridinyl)ethyl]-1H-indene-2-ethanamine [S-(+)-dimethindene] and pharmaceutically acceptable salts and solvates thereof; N,N′-bis [6-[([2-methoxyphenyl)methyl]amino]hexyl]-1,8-octanediamine (methoctramine) and pharmaceutically acceptable salts and solvates thereof; 1,1,24-tris[[5,11-dihydro-6-oxo-6H-pyrido[2,3b][1,4]-benzodiazepin-11-yl)carbonyl]methyl]-8,17-dimethyl-1,8,17,24-tetraazatetracosane (tripitramine) and pharmaceutically acceptable salts and solvates thereof; (3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine) and pharmaceutically acceptable salts and solvates thereof; (3S,3aR,4R,4aS,8aR,9aS)-3-Methyl-4-[2-((R)-1-methyl-6-(S)-methyl-piperidin-2-yl)-vinyl]-decahydro-naphtho [2,3-c]furan-1-one [(+)-himbacine] and pharmaceutically acceptable salts and solvates thereof; (3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof; 4-cyclohexyl-alpha-[4[[4-methoxyphenyl]sulphinyl]-phenyl]-1-piperazineacetonitrile (SCH-57790) and pharmaceutically acceptable salts and solvates thereof; 4-[4-[I(S)-[4-[(1,3-benzodioxol-5-yl)sulfonyl]phenyl]ethyl]-3(R)-methyl-1-piperazinyl]-4-methyl-1-(propylsulfonyl)piperidine (SCH-72788) and pharmaceutically acceptable salts and solvates thereof; 1′-(2-methylbenzoyl)-4-[[[(3,4-methylenedioxyphenyl) sulfonyl]phenyl]methyl]-1,4′-bipiperidine (SCH-76050) and pharmaceutically acceptable salts and solvates thereof; 1′-(2-amino-3-methylbenzoyl)-4-[[[(3-chlorophenyl) sulfonyl]phenyl]methyl]-1,4′-bipiperidine (SCH-211803) and pharmaceutically acceptable salts and solvates thereof; 1′-(2-amino-3-methylbenzoyl)-4-[[[(3-chlorophenyl)sulfonyl]phenyl]ethylenedioxy methyl]-1,4′-bipiperidine (SCH-217443) and pharmaceutically acceptable salts and solvates thereof; 1′-naphto-1-yl-4-[[4-[(methoxycarbonyl)methylthio]phenyl]methyl]-1,4′-bipiperidine (Wang Compound 30) salts and solvates thereof; 1′-(indol-4-yl)carbonyl-4-[[(4-isopropyl)carbonyl]phenyl]methyl]-1,4′-biperidine (Palani Compound 19) and pharmaceutically acceptable salts and solvates thereof; and 1′-(indol-4-yl)carbonyl-4-[[(4-isopropyl)carbonyl]phenyl]ethylenedioxymethyl]-1,4′-biperidine (Palani Compound 30); and
(b) any of the nsPAChAs such as those described herein, each in a pharmaceutical composition in admixture with a pharmaceutical carrier, said nsPAChA being preferably selected from the group consisting of anisotropine pharmaceutically acceptable salts, butylscopolamine pharmaceutically acceptable salts, cimetropium pharmaceutically acceptable salts, clidinium pharmaceutically acceptable salts, fesoterodine and pharmaceutically acceptable salts thereof, glycopyrronium pharmaceutically acceptable salts, otilonium pharmaceutically acceptable salts, oxyphencyclimine and pharmaceutically acceptable salts thereof, prifinium pharmaceutically acceptable salts, propiverine and pharmaceutically acceptable salts thereof, solifenacin and pharmaceutically acceptable salts thereof, tolterodine and pharmaceutically acceptable salts thereof, timepidium pharmaceutically acceptable salts, trospium pharmaceutically acceptable salts, TTS-oxybutynin and pharmaceutically acceptable salts thereof; and valethamate pharmaceutically acceptable salts.

According to this first embodiment, an advantageous combination may be a combination comprising or consisting essentially of
(a) a M₂-antagonist selected from the group consisting of alvameline, as free base or a salt or solvate thereof, especially as its tartrate salt, may be present in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg; in a pharmaceutical composition in admixture with a pharmaceutical carrier or vehicle; and
(b) a nsPAChA, in a pharmaceutical composition in admixture with a pharmaceutical carrier or vehicle.

According to this first embodiment, another advantageous combination may be a combination comprising or consisting essentially of
(a) a M₂-antagonist selected from the group consisting of alvameline, as free base or a salt or solvate thereof, especially as its tartrate, in an amount, in alvameline, of from 200 mg to 600 mg; tripitramine sesquifumarate in an amount, in tripitramine, of from 15 mg to 150 mg; (±)-dimethindene or S(+)-dimethindene maleate, in an amount of from 1.5 mg to 25 mg; otenzepad maleate (1:1), in an amount of from 200 mg to 400 mg; and AQ-RX 741 monomethanesulfonate, in an amount of from 15 mg to 300 mg; in a pharmaceutical composition in admixture with a pharmaceutical carrier or vehicle; and
(b) a nsPAChA in a pharmaceutical composition in admixture with a pharmaceutical carrier or vehicle.

The pharmaceutical combinations of this first embodiment of the present invention, are useful for the treatment of hypocholinergic disorders, and even high doses of a M₂-antagonist Component (a), may be present to improve symptoms of hypocholinergic disorders of the CNS, without adverse effects.

Thus, the present invention provides a method for treating hypocholinergic disorders, which comprises administering to a patient in need of said treatment the combinations described herein in one embodiment. In such a treatment, Component (a), and Component (b) of the combination may be administered simultaneously or sequentially to said patient, Component (a) being indifferently administered before or after Component (b). Components (a) and, Component (b) may also be administered by the same or a different administration route.

According to a second embodiment, the present invention provides a pharmaceutical combination comprising or consisting essentially of, as Components:
(a) a M₂-antagonist, in a pharmaceutical composition in admixture with a pharmaceutical carrier or vehicle; and
(b) a nsPAChA selected from the group consisting of quaternary ammonium nsPAChAs, sulfonium nsPAChAs, solifenacin and pharmaceutically acceptable salts and solvates thereof, propiverine and pharmaceutically acceptable salts and solvates thereof, oxyphencyclimine and pharmaceutically acceptable salts and solvates thereof, tolterodine and pharmaceutically acceptable salts and solvates thereof, fesoterodine and pharmaceutically acceptable salts and solvates thereof, and TTS-oxybutynin; in a pharmaceutical composition in admixture with a pharmaceutical carrier or vehicle.

According to this second embodiment, another combination is a combination comprising or consisting essentially of
(a) a M₂-antagonist, in a pharmaceutical composition in admixture with a pharmaceutical carrier or vehicle; and
(b) quaternary ammonium nsPAChAs of formula I

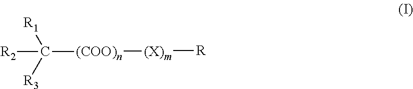

wherein
R is a radical selected from the group consisting of those of formulas (a)-(e)

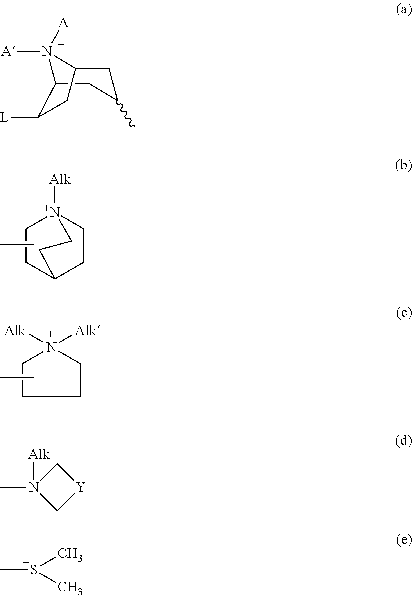

A being methyl and A′ being (C₁-C₄)alkyl or 2-fluoroethyl group or A and A′ forming a 1,4-butylene or 1,5-pentylene chain, L being hydrogen or methoxy, Alk and Alk′ each being (C₁-C₄)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion, such as a chloro, bromo, iodo, tartrate, hydrogen tartrate, succinate, maleate, fumarate, sulfate, hydrogen sulfate or methylsulfate anion;
n and m, independently, are zero or 1;
X is a (C₂-C₃)alkylene group;
R₁ and R₂ are each phenyl, cyclopentyl, cyclohexyl, 1-cyclohexenyl, 2-thienyl and, when R is a radical (a), also each represents (C₁-C₄)alkyl;
R₃ is H or OH or, only when R is a radical (a), also a COOAlk group, Alk being a (C₁-C₄)alkyl group,

in a pharmaceutical composition in admixture with a pharmaceutical carrier.

In particular, in the above formula I at least one of m and n is 1.

Another advantageous combination according to this second embodiment is a combination comprising or consisting essentially of, as Components:
(a) a M₂-antagonist, in a pharmaceutical composition in admixture with a pharmaceutical carrier or vehicle; and
(b) a quaternary ammonium nsPAChA selected form the group consisting of trospium chloride, glycopyrronium bromide, cimetropium bromide, clidinium bromide, otilonium bromide, prifinium bromide, timepidium bromide, scopolamine methobromide, scopolamine butylbromide, scopolamine methonitrate, isopropamide iodide, valethamate bromide, atropine methobromide, atropine methonitrate, diponium bromide, pipenzolate bromide, penthienate bromide, benactizine methobromide, diphemanil, emeprioum bromide and dibutoline sulfate, in a pharmaceutical composition in admixture with a pharmaceutical carrier or vehicle.

Another combination according to this second embodiment is a combination comprising or consisting essentially of, as Components:
(a) a M₂-antagonist, in a pharmaceutical composition in admixture with a pharmaceutical carrier or vehicle; and
(b) a nsPAChA selected from the group consisting of anisotropine methylbromide, in an amount from 25 mg to 300 mg, advantageously from 60 mg to 300 mg, normally from 60 mg to 200 mg; cimetropium bromide, in an amount from 25 mg to 300 mg, advantageously from 60 mg to 300 mg, normally from 60 mg to 200 mg; clidinium bromide, in an amount from 1.25 mg to 15 mg, advantageously from 3 mg to 15 mg, normally from 3 mg to 10 mg; fesoterodine fumarate, in an amount from more than 2 mg to 48 mg, advantageously from 9.6 mg to 48 mg, normally from 9.6 mg to 32 mg; glycopyrronium bromide, in an amount from 1 mg to 16 mg, advantageously from 2.4 mg to 12 mg, normally from 2.4 mg to 8 mg; otilonium bromide, in an amount from 20 mg to 240 mg, advantageously from 48 mg to 240 mg, normally from 48 mg to 160 mg; oxyphencyclimine hydrochloride, in an amount from 5 mg to 60 mg, advantageously from 12 mg to 60 mg, normally from 12 mg to 40 mg; prifinium bromide, in an amount from 15 mg to 180 mg, advantageously from 36 mg to 180 mg, normally from 36 mg to 120 mg; propiverine hydrochloride, in an amount from 7.5 mg to 180 mg, advantageously from 17.5 mg to 180 mg, normally from 17.5 mg to 120 mg; solifenacin succinate, in an amount of 5 mg to 30 mg, advantageously from 12 mg to 30 mg, preferably from 12 mg to 21 mg; timepidium bromide, in an amount from 15 mg to 180 mg, advantageously from 36 mg to 180 mg, normally from 36 mg to 120 mg; trospium chloride, in an amount of from 10 mg to 360 mg, advantageously from 24 mg to 360 mg, normally from 24 mg to 180 mg, TTS-oxybutynin, as a patch releasing from 3.9 mg/24 h to 7.8 mg/24 h, advantageously from 3.9 mg/24 h to 5.85 mg/24 h, normally 3.9 mg/24 h oxybutynin; in a pharmaceutical composition in admixture with a pharmaceutical carrier or vehicle.

A further advantageous combination according to this second embodiment is a combination comprising or consisting essentially of, as Components:
(a) a M₂-antagonist, in a pharmaceutical composition in admixture with a pharmaceutical carrier or vehicle; and
(b) a nsPAChA selected from the group consisting of propiverine hydrochloride, in an amount of from 18 mg to 90 mg in admixture with a pharmaceutical carrier in an IR-formulated composition or in an amount of from 36 mg to 180 mg in admixture with a pharmaceutical carrier in an ER-formulated composition, trospium chloride, in an amount of from 24 mg to 120 mg, normally from 24 mg to 80 mg, in admixture with a pharmaceutical carrier in an IR-formulated composition or in an amount of from 72 mg to 480 mg, normally from 72 mg to 240 mg, in admixture with a pharmaceutical carrier in an ER-formulated composition; TTS-oxybutynin in admixture with a pharmaceutical carrier in a ER-formulated composition consisting of a patch releasing from 3.9 mg/24 h to 7.8 mg/24 h oxybutynin; and solifenacin succinate, in admixture with a pharmaceutical carrier in an IR-formulated composition is preferably present in an amount of from 12 mg to 30 mg or from 12 mg to 21 mg.

In the combinations of the present invention, Component (b) preferably is a nsPAChA selected from the group consisting of anisotropine hydrobromide, in an amount of from 120 mg to 300 mg; butylscopolamine bromide, in an amount of from 12 mg to 40 mg; cimetropium bromide, in an amount of from 55 mg to 200 mg; clidinium bromide, in an amount of from 3 mg to 10 mg; fesoterodine fumarate, in an amount of from 9.6 mg to 32 mg; glycopyrronium bromide, in an amount of from 2.4 mg to 8 mg; otilonium bromide, in an amount of from 48 mg to 160 mg; oxyphencyclimine, in an amount of from 18 mg to 60 mg; prifinium bromide, in an amount of from 36 mg to 120 mg; propiverine hydrochloride IR, in an amount of from 18 mg to 120 mg; solifenacin succinate, in an amount of from 12 mg to 30 mg, normally from 12 mg 21 mg; tolterodine tartrate, in an amount of from 4.8 mg to 16 mg; timepidium bromide, in an amount of from 36 mg to 120 mg; trospium chloride, in an amount of from 24 mg to 240 mg; TTS-oxybutynin in a patch releasing from 3.9 mg/24 h to 7.8 mg/24 h oxybutynin; and valethamate bromide, in an amount of from 12 mg to 40 mg, each nsPAChA being in admixture with a pharmaceutical carrier or vehicle.

The pharmaceutical combinations according to this second embodiment are indicated in the treatment of hypocholinergic disorders and even high doses of a M₂-antagonist Component (a), may be present to improve said symptoms without adverse effects to a greater extent.

Thus, the invention provides a method for treating hypocholinergic disorders, which comprises administering to a patient in need of said treatment the above-illustrated combinations according to this third embodiment. In such a treatment, Component (a) and Component (b) of the combination may be administered simultaneously or sequentially to said patient, Component (a) being indifferently administered before or after Component (b) and Components (a) and Component (b) may also be administered by the same or a different administration route.

According to a third embodiment, an advantageous combination may be a combination comprising or consisting essentially of
(a) a pharmaceutical composition comprising alvameline or a pharmaceutically acceptable salt or solvate thereof, an amount, in alvameline, of from 160 mg to 960 mg, preferably from 240 mg to 960 mg, in admixture with a pharmaceutical carrier or vehicle; and
(b) a pharmaceutical composition comprising nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride; TTS-oxybutynin and valethamate bromide; in admixture with a pharmaceutical carrier or vehicle.

An advantageous combination according to this third embodiment may be a combination comprising or consisting essentially of, as Components:
(a) a pharmaceutical composition comprising alvameline tartrate, in an amount, in alvameline, of from 200 mg to 600 mg, in admixture with a pharmaceutical carrier; and
(b) a pharmaceutical composition comprising a nsPAChA selected from the group consisting of anisotropine hydrobromide, in an amount of from 25 mg to 300 mg, advantageously from 60 mg to 300 mg, normally from 60 mg to 200 mg; butylscopolamine bromide in an amount of from 5 mg to 60 mg, advantageously from 12 mg to 60 mg, normally from 12 mg to 40; cimetropium bromide, in an amount of from 25 mg to 300 mg, advantageously from 60 to 300 mg, normally from 55 mg to 200 mg; clidinium bromide in an amount of from 1.25 mg to 15 mg, advantageously from 3 mg to 15 mg, normally from 3 mg to 12 mg; fesoterodine fumarate, in an amount of from 4 mg to 32 mg, normally from 9.6 mg to 32 mg; glycopyrronium bromide in an amount of from 1 mg to 8 mg, advantageously from 2.2 to 12 mg, normally from 2.2 to 8 mg; otilonium bromide in an amount of from 20 mg to 240 mg, advantageously from 48 mg to 240 mg, normally from 48 mg to 160 mg; prifinium bromide in an amount of from 15 mg to 180 mg, advantageously from 36 mg to 180 mg, normally from 36 mg to 120 mg; propiverine hydrochloride, in an amount of from 7.5 mg to 180 mg, advantageously from 18 mg to 180 mg, normally from 18 mg to 120 mg; solifenacin succinate, in an amount of from 5 mg to 30 mg, advantageously from 12 mg to 30 mg, normally from 12 to 21 mg; tolterodine tartrate, in an amount of from 2 mg to 16 mg, advantageously from 4.8 mg to 24 mg, normally from 4.8 mg to 16 mg; timepidium bromide in an amount of from 15 mg to 180 mg, advantageously from 36 mg to 180 mg, normally from 36 mg to 120 mg; trospium chloride, in an amount of from 10 mg to 360 mg, advantageously from 24 mg to 360 mg, normally from 24 mg to 180 mg, TTS-oxybutynin, as a patch releasing from 3.9 mg/24 h to 7.8 mg/24 h, advantageously from 3.9 mg/24 h to 5.85 mg/24 h, normally 3.9 mg/24 h oxybutynin; and valethamate bromide in an amount of from 5 mg to 60 mg, advantageously from 12 mg to 60 mg, normally from 12 mg to 40 mg;

in admixture with a pharmaceutical carrier or vehicle.

Preferably, according to this third embodiment, the pharmaceutical composition Component (a) comprises alvameline, as free base or as its tartrate, in an amount from 160 mg to 960 mg, in particular, from 160 mg to 480 mg in an IR-formulated oral composition or in an amount of from 240 mg to 960 mg in an ER-formulated composition or device, including a TTS, in admixture with a pharmaceutical carrier or vehicle; and the pharmaceutical composition Component (b) is TTS-oxybutynin, as a patch releasing from 3.9 mg/24 h to 7.8 mg/24 h, normally from 3.9 mg/24 h to 5.85 mg/24 h, preferably 3.9 mg/24 h oxybutynin, in admixture with a pharmaceutical carrier or vehicle.

According to a fourth embodiment, an advantageous M₂-antagonist/nsPAChA combination according to the present invention may be a combination comprising or consisting essentially of

(a) a pharmaceutical composition comprising dimethindene or R-(−)-dimethindene, or a pharmaceutically acceptable salt or solvate thereof, in an amount, in dimethindene or S-(+)-dimethindene, of from 1.1 mg to 32 mg, normally from 1.5 mg to 32 mg, in admixture with a pharmaceutical carrier or vehicle; and
(b) a pharmaceutical composition comprising nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride; TTS-oxybutynin; and valethamate bromide; in admixture with a pharmaceutical carrier or vehicle.

An advantageous combination according to this fourth embodiment may be a combination comprising or consisting essentially of, as Components:
(a) a pharmaceutical composition comprising dimethindene or S-(+)-dimethindene, or a pharmaceutically acceptable salt or solvate thereof, in an amount, in dimethindene or S-(+)-dimethindene, of from 1.1 mg to 32 mg, normally from 1.5 mg to 32 mg, in admixture with a pharmaceutical carrier or vehicle; and
(b) a pharmaceutical composition comprising a nsPAChA selected from the group consisting of anisotropine hydrobromide, in an amount of from 25 mg to 300 mg, advantageously from 60 mg to 300 mg, normally from 60 mg to 200 mg; butylscopolamine bromide in an amount of from 5 mg to 60 mg, advantageously from 12 mg to 60 mg, normally from 12 mg to 40; cimetropium bromide, in an amount of from 25 mg to 300 mg, advantageously from 60 to 300 mg, normally from 55 mg to 200 mg; clidinium bromide in an amount of from 1.5 mg to 15 mg, advantageously from 3 mg to 15 mg, normally from 3 mg to 12 mg; fesoterodine fumarate, in an amount of from 4 mg to 32 mg, normally from 9.6 mg to 32 mg; glycopyrronium bromide in an amount of from 1 mg to 8 mg, advantageously from 2.2 to 12 mg, normally from 2.2 to 8 mg; otilonium bromide in an amount of from 20 mg to 240 mg, advantageously from 48 mg to 240 mg, normally from 48 mg to 160 mg; prifinium bromide in an amount of from 15 mg to 180 mg, advantageously from 36 mg to 180 mg, normally from 36 mg to 120 mg; propiverine hydrochloride, in an amount of from 7.5 mg to 180 mg, advantageously from 18 mg to 180 mg, normally from 18 mg to 120 mg; solifenacin succinate, in an amount of from 5 mg to 30 mg, advantageously from 12 mg to 30 mg, normally from 12 to 21 mg; tolterodine tartrate, in an amount of from 2 mg to 16 mg, advantageously from 4.8 mg to 24 mg, normally from 4.8 mg to 16 mg; timepidium bromide in an amount of from 15 mg to 180 mg, advantageously from 36 mg to 180 mg, normally from 36 mg to 120 mg; trospium chloride, in an amount of from 10 mg to 480 mg, advantageously from 24 mg to 360 mg, normally from 24 mg to 240 mg, TTS-oxybutynin, as a patch releasing from 3.9 mg/24 h to 7.8 mg/24 h, advantageously from 3.9 mg/24 h to 5.85 mg/24 h, normally 3.9 mg/24 h oxybutynin; and valethamate bromide in an amount of from 5 mg to 60 mg, advantageously from 12 mg to 60 mg, normally from 12 mg to 40 mg;

in admixture with a pharmaceutical carrier or vehicle.

Preferably, according to this fourth embodiment, the pharmaceutical composition Component (a) comprises a pharmaceutical composition comprising dimethindene or S-(+)-dimethindene, as free base or as maleate, in an amount, in dimethindene or S-(+)-dimethindene, of from 1.1 mg to 16 mg, normally from 1.5 mg to mg, in admixture with a pharmaceutical carrier or vehicle in an IR-unit form or in an amount of from 4.1 mg to 32 mg, normally from 6 mg to 32 mg, preferably from 6 mg to 24 mg, in admixture with a pharmaceutical carrier or vehicle in a ER-unit form, including TTS forms.

In useful combinations according to this fourth embodiment, the pharmaceutical composition Component (a) comprises a pharmaceutical composition comprising dimethindene or S-(+)-dimethindene, as free base or as maleate, in an amount, in dimethindene or S-(+)-dimethindene, selected from the range group consisting of: from 1.5 mg to 8 mg; from 1.5 mg to 6 mg and from 1.5 mg to 4 mg, in admixture with a pharmaceutical carrier in an IR-unit form; and the pharmaceutical composition Component (b) is TTS-oxybutynin, as a patch releasing from 3.9 mg/24 h to 7.8 mg/24 h, normally from 3.9 mg/24 h to 5.85 mg/24 h, preferably 3.9 mg/24 h oxybutynin, in admixture with a pharmaceutical carrier or vehicle.

In another useful combinations according to this fourth embodiment, the pharmaceutical composition Component (a) comprises a pharmaceutical composition comprising dimethindene or S-(+)-dimethindene, as free base or as maleate, in an amount, in dimethindene or S-(+)-dimethindene, selected from the range group consisting of: from 3 mg to 32 mg; from 4 mg to 32 mg; from 4.4 to 32 mg; from 6 mg to 32 mg; from 6 mg to 16 mg; and from 3 mg to 10 mg, in admixture with a pharmaceutical carrier or vehicle in an ER-form, including a TTS.

According to a fifth embodiment, an advantageous M₂-antagonist/nsPAChA combination may be a combination comprising or consisting essentially of
(a) a pharmaceutical composition comprising otenzepad or a pharmaceutically acceptable salt or solvate thereof, in an amount of 100 mg to 500 mg, normally from 150 mg to 350 mg, in admixture with a pharmaceutical carrier or vehicle; and
(b) a pharmaceutical composition comprising nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride; TTS-oxybutynin; and valethamate bromide; in admixture with a pharmaceutical carrier or vehicle.

According to a further aspect of this fifth embodiment, an advantageous M₂-antagonist/nsPAChA combination may be a combination comprising or consisting essentially of
(a) a pharmaceutical composition comprising otenzepad or a pharmaceutically acceptable salt or solvate thereof, in an amount of 100 mg to 500 mg, normally from 150 mg to 350 mg, in admixture with a pharmaceutical carrier or vehicle; and
(b) a pharmaceutical composition comprising a nsPAChA selected from the group consisting of anisotropine hydrobromide, in an amount of from 25 mg to 300 mg, advantageously from 60 mg to 300 mg, normally from 60 mg to 200 mg; butylscopolamine bromide in an amount of from 5 mg to 60 mg, advantageously from 12 mg to 60 mg, normally from 12 mg to 40; cimetropium bromide, in an amount of from 25 mg to 300 mg, advantageously from 60 to 300 mg, normally from 55 mg to 200 mg; clidinium bromide in an amount of from 1.25 mg to 15 mg, advantageously from 3 mg to 15 mg, normally from 3 mg to 12 mg; fesoterodine fumarate, in an amount of from 4 mg to 32 mg, normally from 9.6 mg to 32 mg; glycopyrronium bromide in an amount of from 1 mg to 8 mg, advantageously from 2.2 to 12 mg, normally from 2.2 to 8 mg; otilonium bromide in an amount of from 20 mg to 240 mg, advantageously from 48 mg to 240 mg, normally from 48 mg to 160 mg; prifinium bromide in an amount of from 15 mg to 180 mg, advantageously from 36 mg to 180 mg, normally from 36 mg to 120 mg; propiverine hydrochloride, in an amount of from 7.5 mg to 180 mg, advantageously from 18 mg to 180 mg, normally from 18 mg to 120 mg; solifenacin succinate, in an amount of from 5 mg to 30 mg, advantageously from 12 mg to 30 mg, normally from 12 mg to 21 mg; tolterodine tartrate, in an amount of from 2 mg to 16 mg, advantageously from 4.8 mg to 24 mg, normally from 4.8 mg to 16 mg; timepidium bromide in an amount of from 15 mg to 180 mg, advantageously from 36 mg to 180 mg, normally from 36 mg to 120 mg; trospium chloride, in an amount of from 10 mg to 360 mg, advantageously from 24 mg to 360 mg, normally from 24 mg to 180 mg, TTS-oxybutynin, as a patch releasing from 3.9 mg/24 h to 7.8 mg/24 h, advantageously from 3.9 mg/24 h to 5.85 mg/24 h, normally 3.9 mg/24 h oxybutynin; and valethamate bromide in an amount of from 5 mg to 60 mg, advantageously from 12 mg to 60 mg, normally from 12 mg to 40 mg;

in admixture with a pharmaceutical carrier or vehicle.

Advantageously, according to this fifth embodiment, the present invention provides a pharmaceutical combination comprises or consist essentially of, as Components:
(a) a M₂-antagonist selected from the group consisting of otenzepad and pharmaceutically acceptable salts thereof in an amount, in otenzepad, of from 100 mg to 500 mg, in a pharmaceutical composition in admixture with a pharmaceutical carrier or vehicle; and
(b) a nsPAChA which is TTS-oxybutynin as a patch releasing from 3.9 mg/24 h to 7.8 mg/24 oxybutynin, wherein oxybutynin is admixture with a pharmaceutical carrier or vehicle.

Preferably, according to this fifth embodiment, the pharmaceutical composition Component (a) comprises a pharmaceutical composition comprising otenzepad, as free base or as the maleate (1:1), fumarate (1:1), dihydrochloride, dihydrochloride dihydrate, dihydrobromide or the monomethanesulfonate thereof, in an amount (in otenzepad) of from 100 mg to 500 mg, preferably from 150 mg to 350 mg in a IR unit form or as the free base or as one of the aforementioned salts, in an amount of from 200 mg to 500 mg, preferably from 300 mg 500 mg, in an ER-form, including a TTS; and the pharmaceutical composition Component (b) is TTS-oxybutynin, as a patch releasing from 3.9 mg/24 h to 7.8 mg/24 h, normally from 3.9 mg/24 h to 5.85 mg/24 h, preferably 3.9 mg/24 h oxybutynin, in admixture with a pharmaceutical carrier or vehicle.

According to a sixth embodiment, an advantageous M₂-antagonist/nsPAChA combination according to the present invention is a combination comprising or consisting essentially of, as Components,
(a) a pharmaceutical composition comprising AQ-RX 741 or a pharmaceutically acceptable salt or solvate thereof, in an amount of from 10 mg to 500 mg, normally from 10 mg to 250 mg, in admixture with a pharmaceutical carrier or vehicle; and
(b) a pharmaceutical composition comprising nsPAChA being preferably selected from the group consisting of anisotropine hydrobromide, butylscopolamine bromide, cimetropium bromide, clidinium bromide, fesoterodine fumarate, glycopyrronium bromide, otilonium bromide, oxyphencyclimine hydrochloride, prifinium bromide, propiverine hydrochloride, solifenacin succinate, tolterodine tartrate, timepidium bromide, trospium chloride; TTS-oxybutynin; and valethamate bromide; in admixture with a pharmaceutical carrier or vehicle.

According to a further aspect of this sixth embodiment, an advantageous M₂-antagonist/nsPAChA combination may be a combination comprising or consisting essentially of
(a) a pharmaceutical composition comprising AQ-RX 741 or a pharmaceutically acceptable salt or solvate thereof, in an amount of from 10 mg to 500 mg, normally from 10 mg to 250 mg, in admixture with a pharmaceutical carrier or vehicle; and
(b) a pharmaceutical composition comprising a nsPAChA selected from the group consisting of anisotropine hydrobromide, in an amount of from 25 mg to 300 mg, advantageously from 60 mg to 300 mg, normally from 60 mg to 200 mg; butylscopolamine bromide in an amount of from 5 mg to 60 mg, advantageously from 12 mg to 60 mg, normally from 12 mg to 40; cimetropium bromide, in an amount of from 25 mg to 300 mg, advantageously from 60 to 300 mg, normally from 55 mg to 200 mg; clidinium bromide in an amount of from 1.5 mg to 15 mg, advantageously from 3 mg to 15 mg, normally from 3 mg to 12 mg; fesoterodine fumarate, in an amount of from 4 mg to 32 mg, normally from 9.6 mg to 32 mg; glycopyrronium bromide in an amount of from 1 mg to 8 mg, advantageously from 2.2 to 12 mg, normally from 2.2 to 8 mg; otilonium bromide in an amount of from 20 mg to 240 mg, advantageously from 48 mg to 240 mg, normally from 48 mg to 160 mg; prifinium bromide in an amount of from 15 mg to 180 mg, advantageously from 36 mg to 180 mg, normally from 36 mg to 120 mg; propiverine hydrochloride, in an amount of from 7.5 mg to 180 mg, advantageously from 18 mg to 180 mg, normally from 18 mg to 120 mg; solifenacin succinate, in an amount of from 5 mg to 30 mg, advantageously from 12 mg to 30 mg, normally from 12 mg to 21 mg; tolterodine tartrate, in an amount of from 2 mg to 16 mg, advantageously from 4.8 mg to 24 mg, normally from 4.8 mg to 16 mg; timepidium bromide in an amount of from 15 mg to 180 mg, advantageously from 36 mg to 180 mg, normally from 36 mg to 120 mg; trospium chloride, in an amount of from 10 mg to 360 mg, advantageously from 24 mg to 360 mg, normally from 24 mg to 180 mg, TTS-oxybutynin, as a patch releasing from 3.9 mg/24 h to 7.8 mg/24 h, advantageously from 3.9 mg/24 h to 5.85 mg/24 h, normally 3.9 mg/24 h oxybutynin; and valethamate bromide in an amount of from 5 mg to 60 mg, advantageously from 12 mg to 60 mg, normally from 12 mg to 40 mg;

in admixture with a pharmaceutical carrier or vehicle.

Advantageously, according to this sixth embodiment, the pharmaceutical composition Component (a) comprises AQ-RX 741 as free base or as monomethanesulfonate, in an amount of from 10 mg to 500 mg, normally from 10 mg to 250 mg, in admixture with a pharmaceutical carrier formulated IR or ER administration. Preferably, Component (a) comprises AQ-RX 741 as free base or as monomethanesulfonate, in an amount of from 10 mg to 500 mg, preferably from 10 mg to 250 mg in a IR-form or, as the free base or the methanesulfonate salt thereof, in an amount of from 20 mg to 500 mg, preferably from 50 mg to 500 mg, in an ER-form, including a TTS; and the pharmaceutical composition Component (b) is TTS-oxybutynin, as a patch releasing from 3.9 mg/24 h to 7.8 mg/24 h, normally from 3.9 mg/24 h to 5.85 mg/24 h, preferably 3.9 mg/24 h oxybutynin, in admixture with a pharmaceutical carrier or vehicle.

In all of these combinations, solifenacin succinate is preferably present as Component (b) in an amount selected from the group consisting of from 5 mg to 30 mg; from 12 mg to 30 mg, and from 12 mg to 21 mg.

Any of the above combinations may contain, as a further component, Component (c), an AChEI also formulated in a pharmaceutical composition, said AChEI may include, but is not limited to, 1,2,3,4-tetrahydro-9-acridinamine (tacrine) and pharmaceutically acceptable salts and solvates thereof, (±)-2,3-dihydro-5,6-dimethoxy-2-[[1-(phenylmethyl)-4-piperidinyl]methyl]-1H-inden-1-one (donepezil) and pharmaceutically acceptable salt and solvates thereof, (S)—N-Ethyl-N-methyl-3-[1-(dimethylamino)ethyl]-phenyl carbamate (rivastigmine) and pharmaceutically acceptable salts and solvates thereof, or 4aS,6R,8aS-3-methoxy-11-methyl-4a,5,9,10,11,12-hexahydroxy-6H-benzofuro[3a,3,2-e,f]benzazepin-6-ol (galantamine) and pharmaceutically acceptable salts and solvates thereof.

Donepezil hydrochloride, available in 5-mg, 10-mg and 23-mg tablets; rivastigmine, preferably as free base or as hydrogen tartrate, available in 1.5-mg, 3-mg and 6-mg, capsules, as a 2-mg/dose oral solution, and in form of a transdermal patch releasing rivastigmine at 4.6 mg/24 hours, 9.5 mg/24 hour or 13.3 mg/24 h; and galantamine, preferably as hydrobromide, available as a 4-mg/ml oral solution, in 4-mg, 8-mg and 12-mg IR-tablets and in 8-mg, 16-mg and 24-mg ER-capsules; are particularly preferred AChEIs.

In said combination, said AChEI Component (c) may be formulated, in admixture with a pharmaceutical carrier or vehicle, in a pharmaceutical composition or device in dosage unit form or also used as a brand preparation.

For example, rivastigmine may be also used by orally administering EXELON® immediate-release 6 mg-capsules or by applying one or more EXELON® patches releasing 4.6 mg/24 hours, 9.5 mg/24 hours, or 13.3 mg/24 hours on the subject's skin, to daily release rivastigmine at a dose/24 h of from 4.6 mg to 53.2 mg or from 19.95 to 53.2 mg, normally from 14.1 mg to 46 m, in combination with the above-illustrated M₂-antagonist/nsPAChA combination.

Donepezil hydrochloride may be also used by orally administering one or more ARICEPT® immediate-release 5 mg- or 10 mg-tablets or the 23-mg tablets. In particular, donepezil hydrochloride may be orally administered, in combination with the above-illustrated M₂-antagonist/nsPAChA combination, at a daily dose of from 5 mg to 100 mg or from 15 mg to 70 mg.

Similarly, galantamine (as hydrobromide) may be also administered as a brand preparation, for example by orally administering RAZADYNE® immediate-release 8 mg- or 12 mg-tablets or RAZADYNE® ER 8 mg-, 16 mg- or 24 mg-capsules.

In particular, galantamine hydrobromide may be orally administered, in combination with the above-illustrated M₂-antagonist/nsPAChA combination, at a daily dose (in galantamine) of from 36 mg to 96 mg, normally at a daily dose or from 36 mg to 72 mg, preferably in an ER-form.

The AChEI Component (c) when included with Component (a) and Component (b) as described herein, may be present in an amount of from about 100% to about 1000% of a recommended dose of Component (c) contained in a unit form used for the treatment of Alzheimer type dementia.

Among the particularly preferred AChEIs, in the combinations of the present invention, as Component (c), donepezil hydrochloride is generally present at a dose of from 5 mg to 98 mg, advantageously from 15 mg to 69 mg, normally from 15 mg to 60 mg; rivastigmine, as hydrogen tartrate, is present, in a composition for oral administration, at a dose in rivastigmine, of from 1.5 mg to 30 mg, advantageously from 6 mg to 24 mg, normally from 9 mg to 18 mg; rivastigmine, as the free base, is present in patch releasing from 4.6 mg/24 h to 52 mg/, advantageously from 9.6 mg/24 h to 39.9 mg/24 h, normally from 13.3 mg/24 h to 39.9 mg/24 h rivastigmine; and galantamine, as hydrobromide, is present in an amount of from 4 mg to 96 mg, advantageously from 12 mg to 96 mg, normally from 18 mg to 48 mg.

Thus, according to another of its aspects, the present invention also provides a pharmaceutical combination comprising or essentially consisting of
(a) a pharmaceutical composition in dosage unit form essentially consisting of a M₂-antagonist, in admixture with a pharmaceutical carrier or vehicle; and
(b) a pharmaceutical composition in dosage unit form essentially consisting of a nsPAChA, in admixture with a pharmaceutical carrier or vehicle; and
(c) an AChEI selected from the group consisting of donepezil hydrochloride in an amount of from 5 mg to 98 mg, advantageously from 15 mg to 69 mg, normally from 15 mg to 60 mg; rivastigmine, as hydrogen tartrate, in an amount, in rivastigmine, of from 1.5 mg to 30 mg, advantageously from 6 mg to 24 mg, normally from 9 mg to 18 mg; rivastigmine, as the free base, in patch releasing from 4.6 mg/24 h to 52 mg/24 h, advantageously from 9.6 mg/24 h to 39.9 mg/24 h, normally from 13.3 mg/24 h to 39.9 mg/24 h or from 4.6 mg/24 h to 13.3 mg/24 h rivastigmine; and galantamine, as hydrobromide, in an amount (in galantamine, of from 4 mg to 96 mg, advantageously from 12 mg to 96 mg, normally from 18 mg to 48 mg, in admixture with a pharmaceutical carrier or vehicle.

According to this aspect, in the above combination the AChEI Component (c) may be combined with any M₂-antagonist Component (a) and with any nsPAChA Component (b) illustrated in this section, in a triple combination useful for combating hypocholinergic disorders of the CNS.

According to this aspect, in the above combination the AChEI Component (c) may be combined with any M₂-antagonist Component (a) and with any nsPAChA Component (b) illustrated in this section, in a triple combination useful for combating hypocholinergic disorders of the CNS. Component (c) may also be combined with Component (b) in an (b/c) fixed dose combination as described for example in U.S. Pat. No. 8,404,701, to be further combined with Component (a). Additionally, Component (c) may be combined with Component (a) in an (a/c) fixed dose combination, to be further combined with Component (b).

### The Combinations in Kits

The present invention also provides a kit or package containing a combination as described herein, accompanied by instructions for use. In particular, a kit of the present invention is a kit comprising a combination of medicaments for the treatment of hypocholinergic disorders of the CNS.

According to the present invention the kit allows for the maximal functional capacity and safety during the treatment of a patient with a combination wherein the components may be administered concurrently or sequentially.

More particularly, the kit of the present invention comprises
(a) a pharmaceutical composition in IR or ER dosage unit form comprising or consisting essentially of a therapeutically effective amount of a M₂-antagonist in admixture with a pharmaceutical carrier or vehicle;
(b) a pharmaceutical composition in IR or ER dosage unit form comprising or consisting essentially of a therapeutically effective amount of a nsPAChA in admixture with a pharmaceutical carrier or vehicle;
   for concurrent, sequential or separate administration.

The pharmaceutical compositions may be packaged in any manner suitable for administration to a patient suffering from a hypocholinergic disorder of the CNS and the packaging is manufactured according to known technologies and completed with instructions for use clearly showing to the patient or to the caregiver how to take each of the units forms to be administered.

Said kit comprises a Component (a) selected among the M₂-antagonists illustrated in the above section “The M₂-antagonists”, and a Component (b) selected among the nsPAChAs illustrated in the above section “The nsPAChAs”.

Component (a) and Component (b) may be present in the kit both in IR or in ER form or one of the Components is in IR form and the other is in ER form, each in admixture with a pharmaceutical carrier or vehicle in a composition formulated as illustrated in “The Formulations” section, according to known technologies.

The kit according to the present invention may also comprise an AChEI Component (c), also in an IR or ER form, in admixture with a pharmaceutical carrier or vehicle in a composition formulated as illustrated in “The Formulations” section below, according to known technologies.

When the AChEI Component (c) is present in the kit, it is in a separate unit form wherein said AChEI is mixed with a pharmaceutical carrier or vehicle in a pharmaceutical composition formulated in an IR or ER unit form

According to a first embodiment, the kit of the present invention comprises
(a) a M₂-antagonist selected from the group consisting of 5-(2-ethyl-2H-tetrazol-5-yl)-1-methyl-1,2, 3,6-tetrahydropyridine (alvameline) and pharmaceutically salts and solvates thereof, 5,11-dihydro-8-chloro-11-[[4-[3-[(2,2-dimethyl-1-oxopentyl)ethylamino]propyl]-1-piperidinyl]acetyl]-6H-pyrido [2,3-b][1,4]benzodiazepin-6-one (BIBN-99) and pharmaceutically acceptable salts and solvates thereof; racemic 11-[[2-(Diethylamino)methyl]-1-piperidinyl]-acetyl]-5,11-dihydro-6H-pyrido[2,3-b][1,4] benzodiazepin-6-one (otenzepad) and pharmaceutically acceptable salts and solvates thereof; dextrorotatory 11-[[2-(diethylamino)methyl]-1-piperidinyl]-acetyl]-5,11-dihydro-6H-pyrido [2,3-b]E1,4] benzodiazepin-6-one [(+)-otenzepad] and pharmaceutically acceptable salts and solvates thereof; N-2-[2-[(dipropylamino)methyl]-1-piperidinyl]ethyl]-5-,6-dihydro-11-H-pyrido[2,3-b][1,4]benzodiazepine-11-carboxamide (AF-DX 384) and pharmaceutically acceptable salts and solvates thereof; 11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof; N,N-Dimethyl-3-[1-(2-pyridinyl)ethyl]-1H-indene-2-ethanamine (dimethindene) and pharmaceutically acceptable salts and solvates thereof; N,N-Dimethyl-3-[(1S)-1-(2-pyridinyl)ethyl]-1H-indene-2-ethanamine [S-(+)-dimethindene] and pharmaceutically acceptable salts and solvates thereof; N,N′-bis[6-[([2-methoxyphenyl)methyl]amino]hexyl]-1,8-octanediamine (methoctramine) and pharmaceutically acceptable salts and solvates thereof; 1,1,24-tris[[5,11-dihydro-6-oxo-6H-pyrido[2,3b][1,4]-benzodiazepin-11-yl)carbonyl] methyl]-8,17-dimethyl-1,8,17,24-tetraazatetracosane (tripitramine) and pharmaceutically acceptable salts and solvates thereof; (3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine) and pharmaceutically acceptable salts and solvates thereof;

(3S,3aR,4R,4aS,8aR,9aS)-3-Methyl-4-[2-((R)-1-methyl-6-(S)-methyl-piperidin-2-yl)-vinyl]-decahydro-naphtho[2,3-c]furan-1-one [(+)-himbacine] and pharmaceutically acceptable salts and solvates thereof; (3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof; 4-cyclohexyl-alpha-[4[[4-methoxyphenyl]sulphinyl]-phenyl]-1-piperazineacetonitrile (SCH-57790) and pharmaceutically acceptable salts and solvates thereof; 4-[4-[1(S)-[4-[(1,3-benzodioxol-5-yl)sulfonyl]phenyl]ethyl]-3(R)-methyl-1-piperazinyl]-4-methyl-1-(propylsulfonyl)piperidine (SCH-72788) and pharmaceutically acceptable salts and solvates thereof; 1′-(2-methylbenzoyl)-4-[[[(3,4-methylenedioxyphenyl) sulfonyl]phenyl]methyl]-1,4′-bipiperidine (SCH-76050) and pharmaceutically acceptable salts and solvates thereof; 1′-(2-amino-3-methylbenzoyl)-4-[[[(3-chlorophenyl)sulfonyl]phenyl]methyl]-1,4′-bipiperidine (SCH-211803) and pharmaceutically acceptable salts and solvates thereof; 1′-(2-amino-3-methylbenzoyl)-4-[[[(3-chlorophenyl)sulfonyl]phenyl]ethylenedioxy methyl]-1,4′-bipiperidine (SCH-217443) and pharmaceutically acceptable salts and solvates thereof; 1′-naphto-1-yl-4-[[4-[(methoxycarbonyl)methylthio]phenyl]methyl]-1,4′-bipiperidine (Wang Compound 30) salts and solvates thereof; 1′-(indol-4-yl)carbonyl-4-[[(4-isopropyl)carbonyl]phenyl]methyl]-1,4′-biperidine (Palani Compound 19) and pharmaceutically acceptable salts and solvates thereof; and 1′-(indol-4-yl)carbonyl-4-[[(4-isopropyl)carbonyl]phenyl]ethylenedioxymethyl]-1,4′-biperidine (Palani Compound 30),

in a pharmaceutical composition in dosage unit form, in admixture with a pharmaceutical carrier or vehicle in an IR- or ER-formulation; and
(b) a nsPAChA selected from the group consisting of quaternary ammonium nsPAChAs, sulfonium nsPAChAs, solifenacin and its pharmaceutically acceptable salts, propiverine and its pharmaceutically acceptable salts, oxyphencyclimine and its pharmaceutically acceptable salts, tolterodine and its pharmaceutically acceptable salts, fesoterodine and its pharmaceutically acceptable salts,
   in a pharmaceutical composition in dosage unit form, in admixture with a pharmaceutical carrier or vehicle in an IR- or ER-formulation.

This kit has the advantage of allowing an improvement in the treatment of a patient suffering from a hypocholinergic disorder. In fact, in the case of the prescription of a M2-antagonist that must be taken three or four times/day the kit of the present invention allows the administration of a composition (b) comprising a nsPAChA that may be administered once a day, thus rendering the treatment easier for the patient or for the caregiver.

A kit according to one aspect of this first embodiment may comprise:

(a) a pharmaceutical composition in dosage unit form comprising or consisting essentially of a M₂-antagonist selected from the group consisting of 5-(2-ethyl-2H-tetrazol-5-yl)-1-methyl-1,2, 3,6-tetrahydropyridine (alvameline) and pharmaceutically salts and solvates thereof, 5,11-dihydro-8-chloro-11-[[4-[3-[(2,2-dimethyl-1-oxopentyl)ethylamino]propyl]-1-piperidinyl]acetyl]-6H-pyrido[2,3-b][1,4]benzodiazepin-6-one (BIBN-99) and pharmaceutically acceptable salts and solvates thereof; racemic 11-[[2-(Diethylamino)methyl]-1-piperidinyl]-acetyl]-5,11-dihydro-6H-pyrido[2,3-b][1,4] benzodiazepin-6-one (otenzepad) and pharmaceutically acceptable salts and solvates thereof; dextrorotatory 11-[[2-(diethylamino)methyl]-1-piperidinyl]-acetyl]-5,11-dihydro-6H-pyrido [2,3-b]E1,4]benzodiazepin-6-one [(+)-otenzepad] and pharmaceutically acceptable salts and solvates thereof; N-2-[2-[(dipropylamino)methyl]-1-piperidinyl]ethyl]-5-,6-dihydro-11-H-pyrido[2,3-b][1,4]benzodiazepine-11-carboxamide (AF-DX 384) and pharmaceutically acceptable salts and solvates thereof; 11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof; N,N-Dimethyl-3-[1-(2-pyridinyl)ethyl]-1H-indene-2-ethanamine (dimethindene) and pharmaceutically acceptable salts and solvates thereof; N,N-Dimethyl-3-[(1S)-1-(2-pyridinyl)ethyl]-1H-indene-2-ethanamine [S-(+)-dimethindene] and pharmaceutically acceptable salts and solvates thereof; N,N′-bis[6-[([2-methoxyphenyl)methyl]amino]hexyl]-1,8-octanediamine (methoctramine) and pharmaceutically acceptable salts and solvates thereof; 1,1,24-tris[[5,11-dihydro-6-oxo-6H-pyrido[2,3b][1,4]-benzodiazepin-11-yl)carbonyl] methyl]-8,17-dimethyl-1,8,17,24-tetraazatetracosane (tripitramine) and pharmaceutically acceptable salts and solvates thereof; (3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine) and pharmaceutically acceptable salts and solvates thereof; (3S,3aR,4R,4aS,8aR,9aS)-3-Methyl-4-[2-((R)-1-methyl-6-(S)-methyl-piperidin-2-yl)-vinyl]-decahydro-naphtho[2,3-c]furan-1-one [(+)-himbacine] and pharmaceutically acceptable salts and solvates thereof; (3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof; 4-cyclohexyl-alpha-[4[[4-methoxyphenyl]sulphinyl]-phenyl]-1-piperazineacetonitrile (SCH-57790) and pharmaceutically acceptable salts and solvates thereof; 4-[4-[1(S)-[4-[(1,3-benzodioxol-5-yl) sulfonyl]phenyl]ethyl]-3(R)-methyl-1-piperazinyl]-4-methyl-1-(propylsulfonyl)piperidine (SCH-72788) and pharmaceutically acceptable salts and solvates thereof; 1′-(2-methylbenzoyl)-4-[[[(3,4-methylenedioxyphenyl)sulfonyl]phenyl]methyl]-1,4′-bipiperidine (SCH-76050) and pharmaceutically acceptable salts and solvates thereof; 1′-(2-amino-3-methylbenzoyl)-4-[[[(3-chlorophenyl) sulfonyl]phenyl]methyl]-1,4′-bipiperidine (SCH-211803) and pharmaceutically acceptable salts and solvates thereof; 1′-(2-amino-3-methylbenzoyl)-4-[[[(3-chlorophenyl)sulfonyl]phenyl]ethylenedioxy methyl]-1,4′-bipiperidine (SCH-217443) and pharmaceutically acceptable salts and solvates thereof; 1′-naphto-1-yl-4-[[4-[(methoxycarbonyl)methylthio]phenyl]methyl]-1,4′-bipiperidine (Wang Compound 30) salts and solvates thereof; 1′-(indol-4-yl)carbonyl-4-[[(4-isopropyl)carbonyl]phenyl]methyl]-1,4′-biperidine (Palani Compound 19) and pharmaceutically acceptable salts and solvates thereof; and 1′-(indol-4-yl)carbonyl-4-[[(4-isopropyl)carbonyl]phenyl]ethylenedioxymethyl]-1,4′-biperidine (Palani Compound 30), in admixture with a pharmaceutical carrier or vehicle in a pharmaceutical composition in an IR- or ER-formulation; and
(b) a pharmaceutical composition in dosage unit form comprising or consisting essentially of a nsPAChA essentially consisting of TTS-oxybutynin, in admixture with a pharmaceutical carrier or vehicle in a pharmaceutical composition in a patch.

According to a second aspect of this first embodiment, a kit of the present invention may comprise:
(a) a pharmaceutical composition in dosage unit form comprising or consisting essentially of a M₂-antagonist selected from the group consisting of alvameline, as free base or a salt or solvate thereof, especially as its tartrate salt, in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg;
   in admixture with a pharmaceutical carrier or vehicle in a pharmaceutical composition in an IR- or ER-formulation; and
(b) a pharmaceutical composition comprising or consisting essentially of a nsPAChA selected from the group consisting of quaternary ammonium nsPAChAs, sulfonium nsPAChAs, (1S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)—N,N-diisopropyl-3-(2-hydroxy-5-methylphenyl)-3-phenylpropanamine (tolterodine) and its pharmaceutically acceptable salts, TTS-oxybutynin, [2-[(1R)-3-(Di(propan-2-yl)amino)-1-phenylpropyl]-4-(hydroxymethyl)phenyl] 2-methylpropanoate (fesoterodine) and its pharmaceutically acceptable salts,
   in admixture with a pharmaceutical carrier or vehicle, in a pharmaceutical composition in an IR- or ER-formulation.

A kit according a second aspect of this first embodiment may comprise:
(a) a pharmaceutical composition in dosage unit form comprising or consisting essentially of a M₂-antagonist selected from the group consisting of alvameline tartrate, in an amount, in alvameline, of from 200 mg to 600 mg; tripitramine sesquifumarate in an amount, in tripitramine, of from 15 mg to 150 mg; (±)-dimethindene or S(+)-dimethindene maleate, in an amount of from 1.5 mg to 25 mg; otenzepad maleate (1:1), in an amount of from 200 mg to 400 mg; and AQ-RX 741 monomethanesulfonate, in an amount of from 15 mg to 300 mg,
   in admixture with a pharmaceutical carrier or vehicle, in a pharmaceutical composition in an IR- or ER-formulation; and
(b) a pharmaceutical composition comprising or consisting essentially of a nsPAChA selected from the group consisting of anisotropine hydrobromide, in an amount of from 25 mg to 400 mg, advantageously from 120 mg to 400 mg, normally from 120 mg to 300 mg; butylscopolamine bromide, in an amount of from 5 mg to 60 mg, advantageously form 12 mg to 60 mg, normally from 12 mg to 40 mg; cimetropium bromide, in an amount of from 25 mg to 200 mg, advantageously from 55 mg to 200 mg; clidinium bromide, in an amount of from 3 mg to 10 mg; fesoterodine fumarate, in an amount of 4 mg to 48 mg, advantageously form 9.6 mg to 48 mg, normally from 9.6 mg to 32 mg; glycopyrronium bromide, in an amount of from 1 mg to 12 mg, advantageously form 2.4 mg to 12 mg, normally from 2.4 mg to 8 mg; otilonium bromide, in an amount of from 20 mg to 240 mg, advantageously form 48 mg to 240 mg, normally from 48 mg to 160 mg; oxyphencyclimine, in an amount of from 5 mg to 60 mg, advantageously form 12 mg to 60 mg, normally from 18 mg to 60 mg; prifinium bromide, in an amount of from 15 mg to 120 mg, advantageously form 24 mg to 120 mg, normally from 36 mg to 120 mg; propiverine hydrochloride, in an amount of from 7.5 mg to 180 mg, advantageously form 18 mg to 180 mg, normally from 18 mg to 120 mg; solifenacin succinate, in an amount of from 5 mg to 30 mg, advantageously form 5 mg to 30 mg, preferably from 12 mg to 30 mg, normally from 12 mg to 21 mg; tolterodine tartrate, in an amount of from 1 mg to 16 mg, advantageously form 2.4 mg to 16 mg, normally from 4.8 mg to 16 mg; timepidium bromide, in an amount of from 15 mg to 180 mg, advantageously form 36 mg to 180 mg, normally from 36 mg to 120 mg; trospium chloride, in an amount of from 10 mg to 480 mg, advantageously from 24 mg to 360 mg, normally from 24 mg to 240 mg; and valethamate bromide, in an amount of from 5 mg to 60 mg, advantageously form 12 mg to 16 mg, normally from 12 mg to 40 mg,
   in admixture with a pharmaceutical carrier or vehicle, in a pharmaceutical composition in an IR- or ER-formulation.

A kit according a third aspect of this first embodiment may comprise:
(a) a pharmaceutical composition in dosage unit form comprising or consisting essentially of a M₂-antagonist selected from the group consisting of alvameline tartrate, in an amount, in alvameline, of from 200 mg to 600 mg; tripitramine sesquifumarate in an amount, in tripitramine, of from 15 mg to 150 mg; (±)-dimethindene or S(+)-dimethindene maleate, in an amount of from 1.5 mg to 25 mg; otenzepad maleate (1:1), in an amount of from 200 mg to 400 mg; and AQ-RX 741 monomethanesulfonate, in an amount of from 15 mg to 300 mg,
   in admixture with a pharmaceutical carrier or vehicle, in a pharmaceutical composition in an IR- or ER-formulation; and
(b) a pharmaceutical composition comprising or consisting essentially of a nsPAChA that is a TTS-oxybutynin consisting of oxybutynin base, in a patch releasing from 3.9 mh/24 h to 7.8 mg/24 h, advantageously from 3.9 mh/24 h to 5.85 mg/24 h, normally of 3.9 mh/24 h,
   in admixture with a pharmaceutical carrier or vehicle,

According to a second embodiment, the invention provides a kit comprising a fixed-dose combination that is a pharmaceutical composition comprising or consisting essentially of

(a) a M₂-antagonist; and
(b) a nsPAChA;
in admixture with a pharmaceutical carrier or vehicle.

An advantageous aspect of this second embodiment provides a kit comprising:

a fixed-dose combination that is a pharmaceutical composition comprising or consisting essentially of
(a) a M₂-antagonist selected from the group consisting of M₂-antagonist being preferably selected from the group consisting of 5-(2-ethyl-2H-tetrazol-5-yl)-1-methyl-1,2, 3,6-tetrahydropyridine (alvameline) and pharmaceutically salts and solvates thereof; 5,11-dihydro-8-chloro-11-[[4-[3-[(2,2-dimethyl-1-oxopentyl)ethylamino]propyl]-1-piperidinyl]acetyl]-6H-pyrido [2,3-b][1,4]benzodiazepin-6-one (BIBN-99) and pharmaceutically acceptable salts and solvates thereof; racemic 11-[[2-(Diethylamino)methyl]-1-piperidinyl]-acetyl]-5,11-dihydro-6H-pyrido[2,3-b][1,4] benzodiazepin-6-one (otenzepad) and pharmaceutically acceptable salts and solvates thereof; dextrorotatory 11-[[2-(diethylamino)methyl]-1-piperidinyl]-acetyl]-5,11-dihydro-6H-pyrido [2,3-b]E1,4] benzodiazepin-6-one [(+)-otenzepad] and pharmaceutically acceptable salts and solvates thereof; N-2-[2-[(dipropylamino)methyl]-1-piperidinyl]ethyl]-5-,6-dihydro-11-H-pyrido[2,3-b][1,4]benzodiazepine-11-carboxamide (AF-DX 384) and pharmaceutically acceptable salts and solvates thereof; 11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof; N,N-Dimethyl-3-[1-(2-pyridinyl)ethyl]-1H-indene-2-ethanamine (dimethindene) and pharmaceutically acceptable salts and solvates thereof; N,N-Dimethyl-3-[(1S)-1-(2-pyridinyl)ethyl]-1H-indene-2-ethanamine [S-(+)-dimethindene] and pharmaceutically acceptable salts and solvates thereof; N,N′-bis [6-[([2-methoxyphenyl)methyl]amino]hexyl]-1,8-octanediamine (methoctramine) and pharmaceutically acceptable salts and solvates thereof; 1,1,24-tris[[5,11-dihydro-6-oxo-6H-pyrido[2,3b][1,4]-benzodiazepin-11-yl)carbonyl] methyl]-8,17-dimethyl-1,8,17,24-tetraazatetracosane (tripitramine) and pharmaceutically acceptable salts and solvates thereof; (3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine) and pharmaceutically acceptable salts and solvates thereof; (3S,3aR,4R,4aS,8aR,9aS)-3-Methyl-4-[2-((R)-1-methyl-6-(S)-methyl-piperidin-2-yl)-vinyl]-decahydro-naphtho [2,3-c]furan-1-one [(+)-himbacine] and pharmaceutically acceptable salts and solvates thereof; (3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof; 4-cyclohexyl-alpha-[4[[4-methoxyphenyl]sulphinyl]-phenyl]-1-piperazineacetonitrile (SCH-57790) and pharmaceutically acceptable salts and solvates thereof; 4-[4-[1(S)-[4-[(1,3-benzodioxol-5-yl)sulfonyl]phenyl]ethyl]-3(R)-methyl-1-piperazinyl]-4-methyl-1-(propylsulfonyl)piperidine (SCH-72788) and pharmaceutically acceptable salts and solvates thereof; 1′-(2-methylbenzoyl)-4-[[[(3,4-methylenedioxyphenyl) sulfonyl]phenyl]methyl]-1,4′-bipiperidine (SCH-76050) and pharmaceutically acceptable salts and solvates thereof; 1′-(2-amino-3-methylbenzoyl)-4-[[[(3-chlorophenyl) sulfonyl]phenyl]methyl]-1,4′-bipiperidine (SCH-211803) and pharmaceutically acceptable salts and solvates thereof; 1′-(2-amino-3-methylbenzoyl)-4-[[[(3-chlorophenyl)sulfonyl]phenyl]ethylenedioxy methyl]-1,4′-bipiperidine (SCH-217443) and pharmaceutically acceptable salts and solvates thereof; 1′-naphto-1-yl-4-[[4-[(methoxycarbonyl)methylthio]phenyl]methyl]-1,4′-bipiperidine (Wang Compound 30) salts and solvates thereof; 1′-(indol-4-yl)carbonyl-4-[[(4-isopropyl)carbonyl]phenyl]methyl]-1,4′-biperidine (Palani Compound 19) and pharmaceutically acceptable salts and solvates thereof; and 1′-(indol-4-yl)carbonyl-4-[[(4-isopropyl)carbonyl]phenyl]ethylenedioxymethyl]-1,4′-biperidine (Palani Compound 30); and
(b) a nsPAChA selected from the group consisting of anisotropine pharmaceutically acceptable salts, butylscopolamine pharmaceutically acceptable salts, cimetropium pharmaceutically acceptable salts, clidinium pharmaceutically acceptable salts, fesoterodine and pharmaceutically acceptable salts thereof, glycopyrronium pharmaceutically acceptable salts, otilonium pharmaceutically acceptable salts, oxyphencyclimine and pharmaceutically acceptable salts thereof, prifinium pharmaceutically acceptable salts, propiverine and pharmaceutically acceptable salts thereof, solifenacin and pharmaceutically acceptable salts thereof, tolterodine and pharmaceutically acceptable salts thereof, timepidium pharmaceutically acceptable salts, trospium pharmaceutically acceptable salts and valethamate pharmaceutically acceptable salts
   in admixture with a pharmaceutical carrier or vehicle.

An advantageous kit according to this second embodiment comprises:
(a) a M₂-antagonist selected from the group consisting of alvameline, as free base or a salt or solvate thereof, especially as its tartrate, in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg; and
(b) a nsPAChA selected from the group consisting of anisotropine hydrobromide, in an amount of from 25 mg to 400 mg, advantageously from 120 mg to 400 mg, normally from 120 mg to 300 mg; butylscopolamine bromide, in an amount of from 5 mg to 60 mg, advantageously form 12 mg to 60 mg, normally from 12 mg to 40 mg; cimetropium bromide, in an amount of from 25 mg to 200 mg, advantageously from 55 mg to 200 mg; clidinium bromide, in an amount of from 1.5 mg to 15 mg, advantageously from 3 mg to 15 mg, normally from 3 mg to 10 mg; fesoterodine fumarate, in an amount of 4 mg to 48 mg, advantageously form 9.6 mg to 48 mg, normally from 9.6 mg to 32 mg; glycopyrronium bromide, in an amount of from 1 mg to 12 mg, advantageously form 2.4 mg to 12 mg, normally from 2.4 mg to 8 mg; otilonium bromide, in an amount of from 20 mg to 240 mg, advantageously form 48 mg to 240 mg, normally from 48 mg to 160 mg; oxyphencyclimine, in an amount of from 5 mg to 60 mg, advantageously form 12 mg to 60 mg, normally from 18 mg to 60 mg; prifinium bromide, in an amount of from 15 mg to 120 mg, advantageously form 24 mg to 120 mg, normally from 36 mg to 120 mg; propiverine hydrochloride, in an amount of from 7.5 mg to 180 mg, advantageously form 18 mg to 180 mg, normally from 18 mg to 120 mg; solifenacin succinate, in an amount of from 5 mg to 30 mg, advantageously form 5 mg to 30 mg, preferably from 12 mg to 30 mg, normally from 12 mg to 21 mg; tolterodine tartrate, in an amount of from 1 mg to 16 mg, advantageously form 2.4 mg to 16 mg, normally from 4.8 mg to 16 mg; timepidium bromide, in an amount of from 15 mg to 180 mg, advantageously form 36 mg to 180 mg, normally from 36 mg to 120 mg; trospium chloride, in an amount of from 10 mg to 480 mg, advantageously from 24 mg to 480 mg, normally 24 mg to 240 mg; and valethamate bromide, in an amount of from 5 mg to 60 mg, advantageously form 12 mg to 16 mg, normally from 12 mg to 40 mg,
   in admixture with a pharmaceutical carrier or vehicle.

Composition (a/b) of the kits of this second embodiment, may be a pharmaceutical composition in dosage unit form comprising or consisting essentially of, as active ingredient, a combination of a M₂-antagonist and of a nsPAChA at specific doses.

More particularly, the present invention provides a pharmaceutical composition in dosage unit form comprising or consisting essentially of

(a) a M₂-antagonist; and
(b) a nsPAChA selected from the group consisting of
anisotropine hydrobromide, in an amount of from 50 mg to 400 mg, advantageously from 120 mg to 400 mg, normally from 120 mg to 300 mg;
butylscopolamine bromide, in an amount of from 5 mg to 60 mg, advantageously from 12 mg to 60 mg, normally from 12 mg to 40 mg;
cimetropium bromide, in an amount of from 25 mg to 300 mg, advantageously from 55 mg to 300 mg, normally from 55 mg to 200 mg;
clidinium bromide, in an amount of from 1.25 mg to 15 mg, advantageously from 3 mg to 15 mg, normally from 3 mg to 10 mg;
fesoterodine fumarate, in an amount of from 4 mg to 48 mg, advantageously from 9.6 mg to 48 mg, normally from 9.6 mg to 32 mg;
glycopyrronium bromide, in an amount of from 1 mg to 12 mg, advantageously from 2.4 mg to 12 mg, normally from 2.4 mg to 8 mg;
otilonium bromide, in an amount of from 20 mg to 180 mg, advantageously from 48 mg to 180 mg, normally from 48 mg to 160 mg;
oxyphencyclimine, in an amount of from 5 mg to 60 mg, advantageously, from 18 mg to 60 mg, normally from 12 mg to 40 mg;
prifinium bromide, in an amount of from 15 mg to 180 mg, advantageously from 36 mg to 180 mg, normally from 36 mg to 120 mg;
propiverine hydrochloride, in an amount of from 7.5 mg to 180 mg, advantageously from 18 mg to 180 mg, normally from 18 mg to 120 mg;
solifenacin succinate, in an amount of from 5 mg to 30 mg, advantageously from 12 mg to 30 mg, normally from 12 mg to 21 mg;
tolterodine hydrogen tartrate, in an amount of from 1 mg to 1 mg to 300 mg, advantageously form 2.4 mg to 16 mg, normally from 4.8 mg to 16 mg;
timepidium bromide, in an amount of from 15 mg to 180 mg, advantageously from 36 mg to 180 mg, normally from 36 mg to 120 mg;
trospium chloride, in an amount of from 10 mg to 240 mg, advantageously form 24 mg to 240 mg normally from 24 mg to 180 mg;
valethamate bromide, in an amount of from 5 mg to 60 mg, advantageously form 12 mg to 16 mg, normally from 12 mg to 40 mg;
   in admixture with a pharmaceutical carrier or vehicle.

According to this second embodiment, the present invention further provides a pharmaceutical composition in dosage unit form comprising or consisting essentially of
(a) a M₂-antagonist selected from the group consisting of alvameline, as free base or a salt or solvate thereof, especially as its tartrate salt, may be present in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg; and
(b) a nsPAChA selected from the group consisting of anisotropine hydrobromide, in an amount of from 50 mg to 400 mg, advantageously from 120 mg to 400 mg, normally from 120 mg to 300 mg; butylscopolamine bromide, in an amount of from 5 mg to 60 mg, advantageously from 12 mg to 60 mg, normally from 12 mg to 40 mg; cimetropium bromide, in an amount of from 25 mg to 300 mg, advantageously from 55 mg to 300 mg, normally from 55 mg to 200 mg; clidinium bromide, in an amount of from 1.25 mg to 15 mg, advantageously from 3 mg to 15 mg, normally from 3 mg to 10 mg; fesoterodine fumarate, in an amount of from 4 mg to 48 mg, advantageously from 9.6 mg to 48 mg, normally from 9.6 mg to 32 mg; glycopyrronium bromide, in an amount of from 1 mg to 12 mg, advantageously from 2.4 mg to 12 mg, normally from 2.4 mg to 8 mg; otilonium bromide, in an amount of from 20 mg to 180 mg, advantageously from 48 mg to 180 mg, normally from 48 mg to 160 mg; oxyphencyclimine, in an amount of from 5 mg to 60 mg, advantageously, from 18 mg to 60 mg, normally from 12 mg to 40 mg; prifinium bromide, in an amount of from 15 mg to 180 mg, advantageously from 36 mg to 180 mg, normally from 36 mg to 120 mg; propiverine hydrochloride, in an amount of from 7.5 mg to 180 mg, advantageously from 18 mg to 180 mg, normally from 18 mg to 120 mg; solifenacin succinate, in an amount of from 5 mg to 30 mg, advantageously from 12 mg to 30 mg, normally from 12 mg to 21 mg; tolterodine tartrate, in an amount of from 1 mg to 1 mg to 300 mg, advantageously form 2.4 mg to 16 mg, normally from 4.8 mg to 16 mg; timepidium bromide, in an amount of from 15 mg to 180 mg, advantageously from 36 mg to 180 mg, normally from 36 mg to 120 mg; trospium chloride, in an amount of from 10 mg to 480 mg, advantageously form 24 mg to 360 mg, normally from 24 mg to 240 mg; and valethamate bromide, in an amount of from 5 mg to 60 mg, advantageously form 12 mg to 16 mg, normally from 12 mg to 40 mg;
   in admixture with a pharmaceutical carrier or vehicle.

In the kits of the present invention, and in the compositions contained therein, solifenacin succinate, when present as Component (b), preferably is in an amount selected from the group consisting of from 5 to 30 mg, from 12 mg to 30 mg, and from 12 mg to 21 mg.

According to a third embodiment of the present invention, each of the above kits may comprise, as a further component of the combinations contained therein, Component (c), an AChEI also formulated in a pharmaceutical composition, said AChEI being selected from the group consisting of 1,2,3,4-tetrahydro-9-acridinamine (tacrine) and pharmaceutically acceptable salts and solvates thereof, (±)-2,3-dihydro-5,6-dimethoxy-2-[[1-(phenylmethyl)-4-piperidinyl]methyl]-1H-inden-1-one (donepezil) and pharmaceutically acceptable salt and solvates thereof, (S)—N-Ethyl-N-methyl-3-[1-(dimethylamino)ethyl]-phenyl carbamate (rivastigmine) and pharmaceutically acceptable salts and solvates thereof, 4aS,6R,8aS-3-methoxy-11-methyl-4a,5,9,10,11,12-hexahydroxy-6H-benzofuro[3a,3,2-e,f]benzazepin-6-ol (galantamine) and pharmaceutically acceptable salts and solvates thereof.

Among the above preferred AChEIs, in the kits of the present invention, as Component (c), donepezil hydrochloride is generally present at a dose of from 5 mg to 98 mg, advantageously from 15 mg to 69 mg, normally from 15 mg to 60 mg; rivastigmine, as hydrogen tartrate, is present, in a composition for oral administration, at a dose in rivastigmine, of from 1.5 mg to 30 mg, advantageously from 6 mg to 24 mg, normally from 9 mg to 18 mg; rivastigmine, as the free base, is present in patch releasing from 4.6 mg/24 h to 52 mg/24 h, advantageously from 9.6 mg/24 h to 39.9 mg/24 h, normally from 13.3 mg/24 h to 39.9 mg/24 h rivastigmine; and galantamine, as hydrobromide, is present in an amount of from 4 mg to 96 mg, advantageously from 12 mg to 96 mg, normally from 18 mg to 48 mg, formulated in a pharmaceutical composition in admixture with a pharmaceutical carrier or vehicle.

Thus, according to one aspect of this third embodiment, the present invention also provides a kit comprising or essentially consisting of
(a) a pharmaceutical composition in dosage unit form essentially consisting of a M₂-antagonist, in admixture with a pharmaceutical carrier or vehicle; and
(b) a pharmaceutical composition in dosage unit form essentially consisting of a nsPAChA, in admixture with a pharmaceutical carrier or vehicle; and
(c) an AChEI selected from the group consisting of donepezil hydrochloride in an amount of from 5 mg to 98 mg, advantageously from 15 mg to 69 mg, normally from 15 mg to 60 mg; rivastigmine, as hydrogen tartrate, in an amount, in rivastigmine, of from 1.5 mg to 30 mg, advantageously from 6 mg to 24 mg, normally from 9 mg to 18 mg; rivastigmine, as the free base, in patch releasing from 4.6 mg/24 h to 52 mg/24 h, advantageously from 9.6 mg/24 h to 39.9 mg/24 h, normally from 13.3 mg/24 h to 39.9 mg/24 h rivastigmine; and galantamine, as hydrobromide, in an amount (in galantamine, of from 4 mg to 96 mg, advantageously from 12 mg to 96 mg, normally from 18 mg to 48 mg,
   in admixture with a pharmaceutical carrier or vehicle.

According to this aspect, in the above combination in the kit, the pharmaceutical composition comprising AChEI Component (c) may be combined with a pharmaceutical composition comprising any M₂-antagonist Component (a) and with a pharmaceutical composition comprising any nsPAChA Component (b) illustrated in this section, in a triple combination useful for combating hypocholinergic disorders of the CNS.

A second aspect of this third embodiment provides a kit comprising:

composition (a/b), a fixed-dose combination that is a pharmaceutical composition comprising or consisting essentially of
(a) a M₂-antagonist selected from the group consisting of alvameline, as free base or a salt or solvate thereof, especially as its tartrate salt, in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg; and
(b) a nsPAChA selected from the group consisting of anisotropine hydrobromide, in an amount of from 50 mg to 400 mg, advantageously from 120 mg to 400 mg, normally from 120 mg to 300 mg; butylscopolamine bromide, in an amount of from 5 mg to 60 mg, advantageously from 12 mg to 60 mg, normally from 12 mg to 40 mg; cimetropium bromide, in an amount of from 25 mg to 300 mg, advantageously from 55 mg to 300 mg, normally from 55 mg to 200 mg; clidinium bromide, in an amount of from 1.25 mg to 15 mg, advantageously from 3 mg to 15 mg, normally from 3 mg to 10 mg; fesoterodine fumarate, in an amount of from 4 mg to 48 mg, advantageously from 9.6 mg to 48 mg, normally from 9.6 mg to 32 mg; glycopyrronium bromide, in an amount of from 1 mg to 12 mg, advantageously from 2.4 mg to 12 mg, normally from 2.4 mg to 8 mg; otilonium bromide, in an amount of from 20 mg to 180 mg, advantageously from 48 mg to 180 mg, normally from 48 mg to 160 mg; oxyphencyclimine, in an amount of from 5 mg to 60 mg, advantageously, from 18 mg to 60 mg, normally from 12 mg to 40 mg; prifinium bromide, in an amount of from 15 mg to 180 mg, advantageously from 36 mg to 180 mg, normally from 36 mg to 120 mg; propiverine hydrochloride, in an amount of from 7.5 mg to 180 mg, advantageously from 18 mg to 180 mg, normally from 18 mg to 120 mg; solifenacin succinate, in an amount of from 5 mg to 30 mg, advantageously from 12 mg to 30 mg, normally from 12 mg to 21 mg; tolterodine tartrate, in an amount of from 1 mg to 1 mg to 300 mg, advantageously form 2.4 mg to 16 mg, normally from 4.8 mg to 16 mg; timepidium bromide, in an amount of from 15 mg to 180 mg, advantageously from 36 mg to 180 mg, normally from 36 mg to 120 mg; trospium chloride, in an amount of from 10 mg to 480 mg, advantageously from 24 mg to 360 mg, normally from 24 mg to 240 mg; and valethamate bromide, in an amount of from 5 mg to 60 mg, advantageously form 12 mg to 16 mg, normally from 12 mg to 40 mg,
   in admixture with a pharmaceutical carrier or vehicle; and.
(c) an AChEI selected from the group consisting of donepezil hydrochloride in an amount of from 5 mg to 98 mg, advantageously from 15 mg to 69 mg, normally from 15 mg to 60 mg; rivastigmine, as hydrogen tartrate, in an amount, in rivastigmine, of from 1.5 mg to 30 mg, advantageously from 6 mg to 24 mg, normally from 9 mg to 18 mg; and galantamine, as hydrobromide, in an amount (in galantamine, of from 4 mg to 96 mg, advantageously from 12 mg to 96 mg, normally from 18 mg to 48 mg,
   in admixture with a pharmaceutical carrier or vehicle.

A particularly preferred form of this second aspect of this third embodiment consists of

composition (a/b), a novel fixed-dose combination that is a pharmaceutical composition in dosage unit form comprising or consisting essentially of
(a) a M₂-antagonist, formulated in a pharmaceutical composition in admixture with a pharmaceutical carrier or vehicle; and
(b) a nsPAChA selected from the group consisting of anisotropine hydrobromide, in an amount of from 60 mg to 300 mg, normally from 60 mg to 200 mg in an IR-formulated oral composition in admixture with a pharmaceutical carrier or vehicle; butylscopolamine bromide in an amount of from 12 mg to 60 mg, normally from 12 mg to 40 mg in an IR-formulated oral composition in admixture with a pharmaceutical carrier or vehicle; cimetropium bromide, in an amount of from 55 mg to 200 mg in an IR-formulated oral composition in admixture with a pharmaceutical carrier or vehicle; clidinium bromide in an amount of from 3 mg to 15 mg, normally from 3 mg to 12 mg in an IR-formulated oral composition in admixture with a pharmaceutical carrier or vehicle; fesoterodine fumarate, in an amount of from 9.6 mg to 32 mg, in an ER-formulated oral composition in admixture with a pharmaceutical carrier or vehicle; glycopyrronium bromide in an amount of from 2.2 to 12 mg, normally from 2.2 to 8 mg in an IR-formulated oral composition in admixture with a pharmaceutical carrier or vehicle; otilonium bromide in an amount of from 48 mg to 240 mg, normally from 48 mg to 160 mg; prifinium bromide in an amount of from 36 mg to 180 mg, normally from 36 mg to 120 mg in an IR-formulated oral composition in admixture with a pharmaceutical carrier or vehicle; propiverine hydrochloride, in an amount of from 18 mg to 90 mg, in an IR-formulated oral composition in admixture with a pharmaceutical carrier or vehicle; propiverine hydrochloride, in an amount of from 36 mg to 180 mg, in an ER-formulated oral composition in admixture with a pharmaceutical carrier or vehicle; solifenacin succinate, in an amount of from 10 mg to 30 mg, normally from 12 to 21 mg, in an IR-formulated oral composition in admixture with a pharmaceutical carrier or vehicle; tolterodine tartrate, in an amount of from 4.8 mg to 16 mg, in an IR-formulated oral composition in admixture with a pharmaceutical carrier or vehicle; timepidium bromide in an amount of from 36 mg to 180 mg, normally from 36 mg to 120 mg in an IR-formulated oral composition in admixture with a pharmaceutical carrier or vehicle; trospium chloride, in an amount of from 24 mg to 80 mg, in an IR-formulated oral composition in admixture with a pharmaceutical carrier or vehicle; trospium chloride, in an amount of from 72 mg to 240 mg, in an ER-formulated oral composition in admixture with a pharmaceutical carrier or vehicle; and valethamate bromide in an amount of from 12 mg to 60 mg, normally from 12 mg to 40 mg, in admixture with a pharmaceutical carrier or vehicle in an oral IR-formulation; and
(c) rivastigmine, as the free base in patch releasing from 4.6 mg/24 h to 52 mg/24 h, advantageously from 9.6 mg/24 h to 39.9 mg/24 h, normally from 13.3 mg/24 h to 39.9 mg/24 h or from 4.6 mg/24 h to 13.3 mg/24 h rivastigmine,
   in admixture with a pharmaceutical carrier or vehicle.

Compositions (a/b) and (c) of the kits of the present invention described above, are novel and a further object of the present invention.

The characteristics, the doses and the use of this pharmaceutical composition are exhaustively illustrated herein above.

According to a third aspect of this third embodiment, the invention also provides a kit comprising
(a) a pharmaceutical composition in dosage unit form comprising or consisting essentially of a M₂-antagomist in admixture with a pharmaceutical carrier or vehicle; and
(b/c) a fixed-dose combination that is a pharmaceutical composition comprising or consisting essentially of a transdermal patch releasing
   (b) a nsPAChA essentially consisting of oxybutynin,
   (c) an AChEI, essentially consisting of rivastigmine,
   in a pharmaceutical composition or device, in admixture with a pharmaceutical carrier or vehicle.

According to this third aspect of this third embodiment, the invention also provides a kit comprising
(a) a pharmaceutical composition in dosage unit form comprising or consisting essentially of a M₂-antagomist in admixture with a pharmaceutical carrier, in an IR-formulated oral composition in admixture with a pharmaceutical carrier or vehicle; and
(b/c) a fixed-dose combination that is a pharmaceutical composition comprising or consisting essentially of a transdermal patch releasing
(b) a nsPAChA essentially consisting of oxybutynin, at a dose of from 3.9 mg/24 h to 7.8 mg/24 h, and
(c) an AChEI, essentially consisting of rivastigmine, at a dose of from 14.63 mg/24 to 133 mg/24 h,
   in a pharmaceutical composition or device, in admixture with a pharmaceutical carrier or vehicle.

According to this third aspect of this third embodiment, the invention further provides a kit comprising
(a) a pharmaceutical composition in dosage unit form comprising or consisting essentially of a M₂-antagomist in admixture with a pharmaceutical carrier; and
(b/c) a fixed-dose combination that is a pharmaceutical composition comprising or consisting essentially of a transdermal patch releasing
(b) a nsPAChA essentially consisting of oxybutynin, at a dose of from 3.9 mg/24 h to 5.85 mg/24 h; and
(c) an AChEI, essentially consisting of rivastigmine, at a dose of from 10.45 mg/24 h to 95 mg/24 h,
   in a pharmaceutical composition or device, in admixture with a pharmaceutical carrier or vehicle.

Finally. according to this third aspect of this third embodiment, the invention provides a kit comprising
(a) a pharmaceutical composition in dosage unit form comprising or consisting essentially of a M₂-antagomist in admixture with a pharmaceutical carrier, in an IR-formulated oral composition in admixture with a pharmaceutical carrier or vehicle; and
(b/c) a fixed-dose combination that is a pharmaceutical composition comprising or consisting essentially of a transdermal patch releasing
(b) a nsPAChA essentially consisting of oxybutynin, at a dose of 3.9 mg/24 h; and
(c) an AChEI, essentially consisting of rivastigmine, at a dose of from 5.06 mg/24 h to 46 mg/24 h,
   in a pharmaceutical composition or device, in admixture with a pharmaceutical carrier or vehicle.

According to this third embodiment, the (b/c) fixed-dose combination is administered to a patient in need of the treatment as a single unit form at the doses illustrated in “The Combinations” section.

This kit has the great advantage of allowing an improvement in the treatment of a patient suffering from a hypocholinergic disorder. In fact, in the case of the prescription of a M₂-antagonist that must be taken three or four times/day the kit of the present invention allows the administration of a composition (b/c) comprising a nsPAChA and an AChEI that may be administered once or twice per day, thus rendering the treatment easier for the patient or for the caregiver.

In instances in which each of the drugs themselves is administered as individual or separate dosage forms (e.g., capsules or tablets), the kit of the present invention comprises each of the drugs making up the composition of the invention, along with instructions for use. Alternatively, each of the drug components of the combination may be combined into a single administrable dosage form such as a capsule.

### The Fixed-Dose Combinations

As indicated above, the pharmaceutical compositions prepared by using the nsPAChAs according to the present invention are present in unit forms also containing a M₂-antagonist that acts by presynaptically releasing acetylcholine in the CNS to improve the symptoms of Alzheimer type dementia.

Thus, it is another object of the present invention to provide a pharmaceutical unit form that comprises
(a) a muscarinic receptor antagonist selected from the group consisting of selective M₂-antagonists; and
(b) a muscarinic receptor antagonist selected from the group consisting of non-selective, peripheral anticholinergic agents (nsPAChAs); and, optionally,
(c) an acetylcholinesterase inhibitor (AChEI),
   in admixture with at least one pharmaceutical carrier or vehicle.

Herein below, the expression “unit form” will also be used to designate a “pharmaceutical composition in dosage unit form”.

The pharmaceutical composition to improve the treatment of human hypocholinergic disorders according to the present invention comprises or consists essentially of a mixture of a M₂-antagonist [Component (a)] and a nsPAChA [Component (b)] wherein Component (a) is present in a quantity sufficient or effective to maximally alleviate disease-associated neurobehavioral symptoms for the treatment of hypocholinergic disorders, in combination with Component (b), surprisingly acting to attenuate the dose-limiting side effects of the M₂-antagonists, thus enabling a greater increase in the MTD of said M₂-antagonists, with attending increase in the therapeutic efficacy of M₂-antagonists. Such a composition allows high doses of M₂-antagonist Component (a) to be safely used, that would have otherwise been dangerous in the absence of Component (b).

The pharmaceutical composition of the present invention improves the treatment of human hypocholinergic disorders of the CNS as described above, such as dementias of the Alzheimer type and schizophrenia.

Any M₂-antagonist and any nsPAChA as described herein, and exemplified in the above “The Combinations” section may be formulated in a pharmaceutical composition in a single unit form, in admixture with at least one pharmaceutical carrier according to conventional methods in the art, and as exemplified in the “The Formulations” section below.

In unit form for immediate release or extended release, the M₂-antagonist Component (a) is present in an amount of from 0.5 mg to 1500 mg. Normally, the M₂-antagonist Component (a) is present, in an IR-form, in an amount of from 0.5 mg to 1000 mg and in an ER-form in an amount of from 1.5 mg to 1500 mg.

Any one of the antagonists of the M₂ receptor subtype illustrated in the above “The M₂-Antagonists” section may be a suitable Component (a), a M₂-antagonist selected from the group consisting of alvameline, BIBN-99, otenzepad, (S)-(+)-otenzepad, AF-DX 384, dimethindene, (S)-(+)-dimethindene, tripitramine, himbacine, (+)-himbacine, the himbacine analog, i.e. the (3aR,4R,4aS,8aR,9aS)-4-{(E)-[(2R,6S)-1,6-dimethylpiperidin-2-yl]ethynyl}-3-methyldecahydronaphtho [2,3-c]furan-1(3H)-one, AQ-RA741, SCH-57790, SCH-72788, SCH-76050, SCH-211803, SCH-217443, Wang Compound 30, Palani Compound 19 and Palani Compound 31 and their pharmaceutically acceptable salts and solvates, as illustrated in the above “The M₂-antagononists” section, being preferable.

According to an embodiment, a M₂-antagonist selected from the group consisting of
alvameline, as free base or a salt or solvate thereof, especially as its tartrate, may be present in an amount, in alvameline, of from 160 mg to 960 mg, preferably from 240 mg to 960 mg;
tripitramine, as free base or a salt or solvate thereof, especially as its sesquifumarate or tetraoxalate salt, in an amount of from 10 mg to 200 mg, preferably from 10 mg to 100 mg in a IR-form or from 25 mg to 200 mg in an ER-form;
dimethindene, preferably as the maleate thereof, as racemate or as its S(+)-enantiomer, in an amount of from 1.2 mg to 30 mg, preferably from 1.2 mg to 15 mg in a IR-form or, as the free base or the maleate thereof, in an amount of from 3 mg to 30 mg, preferably from 3 mg to 10 mg, in an ER-form, including a TTS;
otenzepad, as free base or as the maleate (1:1), fumarate (1:1), dihydrochloride, dihydrochloride dihydrate, dihydrobromide or the monomethanesulfonate (INN: monomesilate, USAN: monomesylate) thereof, in an amount of from 100 mg to 500 mg, preferably from 150 mg to 350 mg in a IR unit form or as the free base or as one of the aforementioned salts, in an amount of from 200 mg to 500 mg, preferably from 300 mg 1500 mg, in an ER-form, including a TTS;
AQ-RX 741, as free base or as a salt or solvate thereof, especially as its monomethanesulfonate salt, in an amount of from 10 mg to 500 mg, preferably from 10 mg to 250 mg in a IR-form or, as the free base or the methanesulfonate salt thereof, in an amount of from 20 mg to 500 mg, preferably from 50 mg to 500 mg, in an ER-form, including a TTS;
   is a particularly preferred Component (a) of the fixed-dose combination.

Any one of the non-selective, muscarinic antagonists that does not appreciably penetrate into the CNS, especially those illustrated in the above “The nsPAChAs” section may be a suitable Component (b).

Advantageous nsPAChAs are solifenacin and its salts, propiverine and its salts, oxyphencyclimine and its salts, tolterodine and its salts, fesoterodine and its salts; and quaternary ammonium salts or sulfonium salts of formula I above, such as homatropine quaternary salts, anisotropine quaternary salts, trospium quaternary salts, clidinium quaternary salts, benzilonium quaternary salts and glycopyrronium quaternary salts. Other suitable quaternary ammonium salts are scopolamine methobromide, scopolamine butylbromide, scopolamine methonitrate, isopropamide iodide, valethamate bromide, atropine methobromide, atropine methonitrate, diponium bromide, pipenzolate bromide, penthienate bromide, benactizine methobromide, diphemanil, emeprioum bromide and dibutoline sulfate.

Anisotropine hydrobromide; butylscopolamine bromide; cimetropium bromide; clidinium bromide; glycopyrronium bromide; methylpropiverinium iodide or bromide; otilonium bromide; prifinium bromide; timepidium bromide; trospium chloride, succinate, maleate, fumarate or tartrate; valethamate bromide; fesoterodine and its fumarate; oxyphencyclimine and its hydrochloride; propiverine and its hydrochloride; solifenacin and its succinate; tolterodine and the L-hydrogen tartrate thereof are particularly advantageous nsPAChAs used as Component (b).

In the unit forms of the present invention, for immediate release or extended release, the nsPAChA Component (b) is generally present in an amount of from 50% to 600%, preferably from 1.2-fold to 6 times the maximum IR amount of said nsPAChA contained in the currently administered IR dosage unit forms used in the anticholinergic therapy.

Normally, but not necessarily, the nsPAChA is generally present, in an IR unit form, in an amount ranging from 50% to 400%, preferably from 120% to 400%, the maximum amount of said nsPAChA contained in the currently administered IR dosage unit forms for the anticholinergic therapy or, in an ER unit form, in an amount ranging from 75% to 600%, preferably from 120% to 600%, the maximum amount of said nsPAChA contained in the currently administered unit dosage IR forms for the anticholinergic therapy.

For example, among the nsPAChAs used as Component (b),
anisotropine hydrobromide is present in an amount of from 25 mg to 300 mg, in IR or ER form, preferably from 60 mg to 200 mg in IR form;
butylscopolamine bromide is present in an amount of from 5 mg to 60 mg in IR or ER form, preferably from 12 mg to 40 mg in IR form;
cimetropium bromide is present in an amount of from 25 mg to 300 mg in IR or ER form, preferably from 60 mg to 200 mg in IR form;
clidinium bromide is present in an amount of from 1.25 mg to 15 mg in IR or ER form, preferably from 3 mg to 10 mg in IR form;
fesoterodine fumarate is present in an amount of from 4 mg to 32 mg, preferably from 9.6 mg to 32 mg in ER form;
glycopyrronium bromide is present in an amount of from 1-24 mg to 8 mg in IR or ER form, preferably from 2.4 mg to 4 mg in IR form;
otilonium bromide is present in an amount of from 48 mg to 160 mg in IR or ER form, preferably from 48 mg to 120 mg in IR form;
oxyphencyclimine is present in an amount of from 5 mg to 60 mg, advantageously from 18 mg to 60 mg in IR or ER form, preferably from 18 mg to 40 mg in IR form;
prifinium bromide is present in an amount of from 36 mg to 120 mg in IR or ER form, preferably from 36 mg to 120 mg in IR form;
propiverine hydrochloride is present in an amount of from 7.5 mg to 180 mg, preferably from 18 mg to 60 mg in IR form and from 36 mg to 180 mg in ER form;
solifenacin succinate is present in an amount of from 5 mg to 30 mg, normally from 12 mg to 30 mg or from 12 mg to 21 mg in IR form;
tolterodine hydrogen tartrate is present in an amount of from 2 mg to 24 mg, in IR or ER form, preferably from 4.8 mg to 16 mg in IR form;
timepidium bromide is present in an amount of from 15 mg to 180 mg in IR or ER form, preferably from 36 mg to 120 mg in IR form;
trospium chloride IR is present in an amount of from 10 mg to 480 mg, advantageously from 10 mg to 240 mg in an IR or ER form, preferably from 24 mg to 80 mg in IR form and from 72 mg to 240 mg in ER form; and
valethamate bromide is present in an amount of from 5 mg to 60 mg in IR or ER form, preferably from 12 mg to 40 mg in IR form.

Any one of the Components (a) may be combined with any one Component (b) in a fixed-dose combination comprising said Component (a) and said Component (b) in a pharmaceutical composition in dosage unit form in admixture with a pharmaceutical carrier or vehicle for IR or ER administration.

In particular, the fixed-dose combination of the invention consists of a pharmaceutical composition in dosage unit form comprising or consisting essentially of
(a) any of the M₂-antagonists as illustrated in the above “The M₂-antagonists” section, each in a pharmaceutical composition in admixture with a pharmaceutical carrier, said M₂-antagonist being preferably selected from the group consisting of 5-(2-ethyl-2H-tetrazol-5-yl)-1-methyl-1,2,3,6-tetrahydropyridine (alvameline) and pharmaceutically salts and solvates thereof, 5,11-dihydro-8-chloro-11-[[4-[3-[(2,2-dimethyl-1-oxopentyl)ethylamino]propyl]-1-piperidinyl]acetyl]-6H-pyrido[2,3-b][1,4]benzodiazepin-6-one (BIBN-99) and pharmaceutically acceptable salts and solvates thereof; racemic 11-[[2-(Diethylamino)methyl]-1-piperidinyl]-acetyl]-5,11-dihydro-6H-pyrido[2,3-b][1,4] benzodiazepin-6-one (otenzepad) and pharmaceutically acceptable salts and solvates thereof; dextrorotatory 11-[[2-(diethylamino)methyl]-1-piperidinyl]-acetyl]-5,11-dihydro-6H-pyrido[2,3-b]E1,4] benzodiazepin-6-one [(+)-otenzepad] and pharmaceutically acceptable salts and solvates thereof; N-2-[2-[(dipropylamino)methyl]-1-piperidinyl]ethyl]-5-,6-dihydro-11-H-pyrido[2,3-b][1,4]benzodiazepine-11-carboxamide (AF-DX 384) and pharmaceutically acceptable salts and solvates thereof; 11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof; N,N-Dimethyl-3-[1-(2-pyridinyl)ethyl]-1H-indene-2-ethanamine (dimethindene) and pharmaceutically acceptable salts and solvates thereof; N,N-Dimethyl-3-[(1S)-1-(2-pyridinyl)ethyl]-1H-indene-2-ethanamine [S-(+)-dimethindene] and pharmaceutically acceptable salts and solvates thereof; N,N′-bis [6-[([2-methoxyphenyl)methyl]amino]hexyl]-1,8-octanediamine (methoctramine) and pharmaceutically acceptable salts and solvates thereof; 1,1,24-tris[[5,11-dihydro-6-oxo-6H-pyrido[2,3b][1,4]-benzodiazepin-11-yl)carbonyl] methyl]-8,17-dimethyl-1,8,17,24-tetraazatetracosane (tripitramine) and pharmaceutically acceptable salts and solvates thereof; (3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine) and pharmaceutically acceptable salts and solvates thereof;

(3S,3aR,4R,4aS,8aR,9aS)-3-Methyl-4-[2-((R)-1-methyl-6-(S)-methyl-piperidin-2-yl)-vinyl]-decahydro-naphtho[2,3-c]furan-1-one [(+)-himbacine] and pharmaceutically acceptable salts and solvates thereof; (3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof; 4-cyclohexyl-alpha-[4 [[4-methoxyphenyl]sulphinyl]-phenyl]-1-piperazineacetonitrile (SCH-57790) and pharmaceutically acceptable salts and solvates thereof; 4-[4-[1(S)-[4-[(1,3-benzodioxol-5-yl) sulfonyl]phenyl]ethyl]-3(R)-methyl-1-piperazinyl]-4-methyl-1-(propylsulfonyl)piperidine (SCH-72788) and pharmaceutically acceptable salts and solvates thereof; 1′-(2-methylbenzoyl)-4-[[[(3,4-methylenedioxyphenyl) sulfonyl]phenyl]methyl]-1,4′-bipiperidine (SCH-76050) and pharmaceutically acceptable salts and solvates thereof; 1′-(2-amino-3-methylbenzoyl)-4-[[[(3-chlorophenyl) sulfonyl]phenyl]methyl]-1,4′-bipiperidine (SCH-211803) and pharmaceutically acceptable salts and solvates thereof; 1′-(2-amino-3-methylbenzoyl)-4-[[[(3-chlorophenyl)sulfonyl]phenyl]ethylenedioxy methyl]-1,4′-bipiperidine (SCH-217443) and pharmaceutically acceptable salts and solvates thereof; 1′-naphto-1-yl-4-[[4-[(methoxycarbonyl)methylthio]phenyl]methyl]-1,4′-bipiperidine (Wang Compound 30) salts and solvates thereof; 1′-(indol-4-yl)carbonyl-4-[[(4-isopropyl)carbonyl]phenyl]methyl]-1,4′-biperidine (Palani Compound 19) and pharmaceutically acceptable salts and solvates thereof; and 1′-(indol-4-yl)carbonyl-4-[[(4-isopropyl)carbonyl]phenyl]ethylenedioxymethyl]-1,4′-biperidine (Palani Compound 30); and
(b) any of the nsPAChAs as illustrated in the above “The nsPAChAs” section, said nsPAChA being preferably selected from the group consisting of anisotropine pharmaceutically acceptable quaternary salts, butylscopolamine pharmaceutically acceptable salts, cimetropium pharmaceutically acceptable salts, clidinium pharmaceutically acceptable salts, fesoterodine and pharmaceutically acceptable salts thereof, glycopyrronium pharmaceutically acceptable salts, otilonium pharmaceutically acceptable salts, oxyphencyclimine and pharmaceutically acceptable salts thereof, prifinium pharmaceutically acceptable salts, propiverine and pharmaceutically acceptable salts thereof, solifenacin and pharmaceutically acceptable salts thereof, tolterodine and pharmaceutically acceptable salts thereof, timepidium pharmaceutically acceptable salts, trospium pharmaceutically acceptable salts; TTS-oxybutynin; and valethamate pharmaceutically acceptable salts;
   in admixture with at least one pharmaceutical carrier or vehicle.

According to an embodiment, an advantageous fixed-dose combination consists of a pharmaceutical composition comprising or consisting essentially of
(a) a M₂-antagonist selected from the group consisting of alvameline, as free base or a salt or solvate thereof, especially as its tartrate salt, may be present in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg; and
(b) a nsPAChA selected from the group consisting of
   anisotropine hydrobromide, in an amount of from 25 mg to 300 mg, normally from 60 mg to 200 mg;
   butylscopolamine bromide in an amount of from 5 mg to 60 mg, normally from 12 mg to 40 mg;
   cimetropium bromide, in an amount of from 25 mg to 200 mg, normally from 55 mg to 200 mg;
   clidinium bromide in an amount of from 1.25 mg to 15 mg, normally from 3 mg to 12 mg;
   otilonium bromide in an amount of from 20 mg to 240 mg, normally from 48 mg to 160 mg;
   prifinium bromide in an amount of from 15 mg to 180 mg, normally from 36 mg to 180 mg;
   timepidium bromide in an amount of from 15 mg to 180 mg, normally from 36 mg to 120 mg;
   valethamate bromide in an amount of from 5 mg to 60 mg, normally from 12 mg to 40 mg
      said composition being formulated in an IR dosage unit form in admixture with a pharmaceutical carrier or vehicle.

According to another embodiment, a particularly advantageous fixed-dose combination consists of a pharmaceutical unit form comprising or consisting essentially of
(a) alvameline, as free base or a salt or solvate thereof, especially as its tartrate salt, may be present in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg; and
(b) a nsPAChA selected from the group consisting of
   fesoterodine fumarate, in an amount of from 4 mg to 32 mg, normally from 9.6 mg to 32 mg;
   propiverine hydrochloride, in an amount of from 7.5 mg to 180 mg, normally from 36 mg to 180 mg;
   solifenacin succinate, in an amount of from 10 mg to 30 mg, advantageously from 12 mg to 30 mg, normally from 12 mg to 21 mg;
   tolterodine tartrate, in an amount of from 2 mg to 16 mg, normally from 4.8 mg to 16 mg;
   trospium chloride, in an amount of from 10 mg to 480 mg, normally from 10 mg to 240 mg, preferably from 72 mg to 240 mg
      in admixture with a pharmaceutical carrier or vehicle.

According to a further embodiment, a particularly advantageous fixed-dose combination consists of a pharmaceutical dosage unit form comprising or consisting essentially of
(a) alvameline, as free base or a salt or solvate thereof, especially as its tartrate salt, may be present in an amount, in alvameline, of from 160 mg to 960 mg, preferably from 240 mg to 960 mg; tripitramine, as free base or a salt or solvate thereof, especially as its sesquifumarate or tetraoxalate salt, in an amount, in tripitramine, of from 10 mg to 200 mg, preferably from 25 mg to 100 mg; (±)-dimethindene or S(+)-dimethindene, in an amount of from 1.1 mg to 32 mg, preferably from 1.2 mg to 15 mg; otenzepad, as free base or as the maleate (1:1), fumarate (1:1), dihydrochloride, dihydrochloride dihydrate, dihydrobromide or the monomethanesulfonate, in an amount of from 100 mg to 500 mg, preferably from 150 mg to 350 mg; and AQ-RX 741, as free base or as a salt or solvate thereof, especially as its monomethanesulfonate salt, in an amount of from 10 mg to 500 mg, preferably from 10 mg to 250 mg;
(b) propiverine, as a salt thereof such as its hydrochloride, in an amount of from 7.5 mg to 180 mg, advantageously from 31 to 120 mg, normally from 45 mg to 90 mg;
   in admixture with at least one pharmaceutical carrier or vehicle for oral administration.

According to the present invention each of the above fixed-dose combinations may include, as a further component (c), an AChEI also formulated in a pharmaceutical composition, said AChEI being preferably selected from the group consisting of 1,2,3,4-tetrahydro-9-acridinamine (tacrine) and pharmaceutically acceptable salts and solvates thereof, (±)-2,3-dihydro-5,6-dimethoxy-2-[[1-(phenylmethyl)-4-piperidinyl]methyl]-1H-inden-1-one (donepezil) and pharmaceutically acceptable salt and solvates thereof, (S)—N-Ethyl-N-methyl-3-[1-(dimethylamino)ethyl]-phenyl carbamate (rivastigmine) and pharmaceutically acceptable salts and solvates thereof, 4aS,6R,8aS-3-methoxy-11-methyl-4a,5,9,10,11,12-hexahydroxy-6H-benzofuro[3a,3,2-e,f]benzazepin-6-ol (galantamine) and pharmaceutically acceptable salts and solvates thereof.

Thus, the present invention also provides fixed-dose combinations essentially consisting of a pharmaceutical composition in dosage unit form comprising

(a) a M₂-antagonist;
(b) a nsPAChA; and

### (c) an AChEI;

in admixture with a pharmaceutical carrier or vehicle.

For example, the pharmaceutical composition of the present invention comprises:

(a) a M₂-antagonist selected from the group consisting of 5-(2-ethyl-2H-tetrazol-5-yl)-1-methyl-1,2, 3,6-tetrahydropyridine (alvameline) and pharmaceutically salts and solvates thereof, 5,11-dihydro-8-chloro-11-[[4-[3-[(2,2-dimethyl-1-oxopentyl)ethylamino]propyl]-1-piperidinyl]acetyl]-6H-pyrido [2,3-b][1,4]benzodiazepin-6-one (BIBN-99) and pharmaceutically acceptable salts and solvates thereof; racemic 11-[[2-(Diethylamino)methyl]-1-piperidinyl]-acetyl]-5,11-dihydro-6H-pyrido[2,3-b][1,4] benzodiazepin-6-one (otenzepad) and pharmaceutically acceptable salts and solvates thereof; dextrorotatory 11-[[2-(diethylamino)methyl]-1-piperidinyl]-acetyl]-5,11-dihydro-6H-pyrido [2,3-b]E1,4]benzodiazepin-6-one [(+)-otenzepad] and pharmaceutically acceptable salts and solvates thereof; N-2-[2-[(dipropylamino)methyl]-1-piperidinyl]ethyl]-5-,6-dihydro-11-H-pyrido[2,3-b][1,4]benzodiazepine-11-carboxamide (AF-DX 384) and pharmaceutically acceptable salts and solvates thereof; 11-[[4-[4-(Diethylamino)butyl]-1-piperidinyl]acetyl]-5,11-dihydro-6H-pyrido[2,3-b][1,4]benzodiazepin-6-one (AQ-RA 741) and pharmaceutically acceptable salts and solvates thereof; N,N-Dimethyl-3-[1-(2-pyridinyl)ethyl]-1H-indene-2-ethanamine (dimethindene) and pharmaceutically acceptable salts and solvates thereof; N,N-Dimethyl-3-[(1S)-1-(2-pyridinyl)ethyl]-1H-indene-2-ethanamine [S-(+)-dimethindene] and pharmaceutically acceptable salts and solvates thereof; N,N′-bis[6-[([2-methoxyphenyl)methyl]amino]hexyl]-1,8-octanediamine (methoctramine) and pharmaceutically acceptable salts and solvates thereof; 1,1,24-tris[[5,11-dihydro-6-oxo-6H-pyrido[2,3b][1,4]-benzodiazepin-11-yl)carbonyl] methyl]-8,17-dimethyl-1,8,17,24-tetraazatetracosane (tripitramine) and pharmaceutically acceptable salts and solvates thereof; (3aR,4R,4aS,8aR,9aS)-4-{(E)-2-[(2R,6S)-1,6-dimethylpiperidin-2-yl]ethenyl}-3-methyldecahydronaphtho [2,3-c]furan-1 (3H)-one (himbacine) and pharmaceutically acceptable salts and solvates thereof; (3S,3aR,4R,4aS,8aR,9aS)-3-Methyl-4-[2-((R)-1-methyl-6-(S)-methyl-piperidin-2-yl)-vinyl]-decahydro-naphtho[2,3-c]furan-1-one [(+)-himbacine] and pharmaceutically acceptable salts and solvates thereof; (3aR,4R,4aS,8aR,9aS)-4-{(E)-2-[(2R,6S)-1,6-dimethylpiperidin-2-yl]ethynyl}-3-methyldecahydronaphtho [2,3-c]furan-1 (3H)-one (himbacine analog) and pharmaceutically acceptable salts and solvates thereof; 4-cyclohexyl-alpha-[4[[4-methoxyphenyl]sulphinyl]-phenyl]-1-piperazineacetonitrile (SCH-57790) and pharmaceutically acceptable salts and solvates thereof; 4-[4-[1(S)-[4-[(1,3-benzodioxol-5-yl)sulfonyl]phenyl]ethyl]-3(R)-methyl-1-piperazinyl]-4-methyl-1-(propylsulfonyl)piperidine (SCH-72788) and pharmaceutically acceptable salts and solvates thereof; 1′-(2-methylbenzoyl)-4-[[[(3,4-methylenedioxyphenyl)sulfonyl]phenyl]methyl]-1,4′-bipiperidine (SCH-76050) and pharmaceutically acceptable salts and solvates thereof; 1′-(2-amino-3-methylbenzoyl)-4-[[[(3-chlorophenyl) sulfonyl]phenyl]methyl]-1,4′-bipiperidine (SCH-211803) and pharmaceutically acceptable salts and solvates thereof; 1′-(2-amino-3-methylbenzoyl)-4-[[[(3-chlorophenyl)sulfonyl]phenyl]ethylenedioxy methyl]-1,4′-bipiperidine (SCH-217443) and pharmaceutically acceptable salts and solvates thereof; 1′-naphto-1-yl-4-[[4-[(methoxycarbonyl)methylthio]phenyl]methyl]-1,4′-bipiperidine (Wang Compound 30) salts and solvates thereof; 1′-(indol-4-yl)carbonyl-4-[[(4-isopropyl)carbonyl]phenyl]methyl]-1,4′-biperidine (Palani Compound 19) and pharmaceutically acceptable salts and solvates thereof; and 1′-(indol-4-yl)carbonyl-4-[[(4-isopropyl)carbonyl]phenyl]ethylenedioxymethyl]-1,4′-biperidine (Palani Compound 30);
(b) a nsPAChA selected from the group consisting of quaternary ammonium nsPAChAs, sulfonium nsPAChAs, solifenacin and its pharmaceutically acceptable salts, propiverine and its pharmaceutically acceptable salts, oxyphencyclimine and its pharmaceutically acceptable salts, tolterodine and its pharmaceutically acceptable salts, TTS-oxybutynin, fesoterodine and its pharmaceutically acceptable salts; and
(c) an AChEI selected from the group consisting of donepezil and its pharmaceutically acceptable salts, rivastigmine and its pharmaceutically acceptable salts, and galantamine and its pharmaceutically acceptable salts, in admixture with a pharmaceutical carrier or vehicle.

In this fixed-dose combination, when Component (b) is oxybutynin in a TTS, also the M₂-antagonist Component (a) and the AChEI Component (c) are included in the same TTS:

The above fixed-dose combination and any of the pharmaceutical compositions that are part of the above combinations and kits are formulated with pharmaceutical carriers, diluents, vehicles and devices according to known and conventional methods and/or technologies in the art and as illustrated in the “The Formulations” section below. In addition, any of the above fixed-dose combination and any of the above pharmaceutical compositions may further include a Component (c) an AChEI, as illustrated herein above.

Among the preferred AChEIs, in the combinations of the present fixed-dose combinations, donepezil hydrochloride is present at a dose of from 5 mg to 98 mg, advantageously from 10 mg to 98 mg, preferably from 15 mg to 69 mg, normally from 15 mg to 60 mg; rivastigmine, as hydrogen tartrate, is present, in a composition for oral administration, at a dose, in rivastigmine, of from 1.5 mg to 30 mg, advantageously from 6 mg to 30 mg, preferably from 9 mg to 24 mg, normally from 9 mg to 18 mg; rivastigmine, as the free base, is present in patch releasing from 4.6 mg/24 h to 52 mg/24 h, advantageously from 9.6 mg/24 h to 39.9 mg/24 h, normally from 13.3 mg/24 h to 39.9 mg/24 h or from 4.6 mg/24 h to 13.3 mg/24 h rivastigmine; and galantamine, as hydrobromide, is present in an amount of from 4 mg to 96 mg, advantageously from 12 mg to 96 mg, normally from 18 mg to 48 mg.

### The Formulations

The unit form of the present invention may be a tablet, a capsule, a pre-measured volume of a liquid solution or suspension for oral administration or a TTS as a gel or patch for transdermal application. In said unit form the M₂-antagonist and the nsPAChA, as free base or as a pharmaceutically acceptable salt or solvate thereof, may be mixed together or separated according to known technologies in admixture with a pharmaceutical carrier in a pharmaceutical composition.

Component (a) and Component (b), and optionally a further Component (c), are formulated with conventional pharmaceutical carriers in known formulations for oral use wherein said components are mixed together or separated, for example in two or three tablets introduced in a capsule or in a two-compartment capsule, wherein one of the Components (a) and (b), is in a first of the two compartments and the other is in the second of the two compartments, or in a multilayer (di-layer) tablet wherein the two components are both in IR or in ER form or one of the two components is in IR form and the other is in ER form, according to known technologies. Component (c) may be optionally included in the first or second compartment, or optionally included in a multilayer tablet as described herein above, with one or more of Components (a) and (b).

The pharmaceutical carriers and vehicles are those commonly used for the preparation of compositions for oral, buccal and parenteral, in particular transdermal, administration. Appropriate unit forms comprise the oral forms such as tablets, soft or hard gelatin capsules, powders or granulates in sachets and suitably measured oral solutions or suspensions as well as patches for transdermal administration.

Component (a) and Component (b), with optionally a further Component (c), may also be present in form of one of their complexes with a cyclodextrin, for example α-cyclodextrin, β-cyclodextrin, γ-cyclodextrin, 2-hydroxypropyl-β-cyclodextrin or methyl-β-cyclodextrin.

Component (a) and Component (b), with optionally a further Component (c), may also be formulated in the form of microcapsules, optionally with one or more carriers or additives.

For oral administration, Component (a) and Component (b), with optionally a further Component (c), together or separately, are formulated by mixing the active ingredient with conventional pharmaceutical acceptable carriers enabling said active ingredients to be formulated in tablets, dragees, orally disintegrating tablets, capsules, liquid solutions or suspensions, syrups and the like.

Carriers for IR tablets include for example starches, cellulose and derivatives thereof; lubricants such as talc, stearic acid or magnesium stearate; diluents such as talc, powdered cellulose, lactose, starches such as maize or corn starch, mannitol, sorbitol; disaggregating agents such as microcrystalline cellulose or crospovidone; lubricants such as polyethylene glycol or magnesium stearate; ligands such as methylcellulose, sodium carboxymethylcellulose, alginic acid, alginates; sweeteners, such as sucrose, dextrose, mannitol, saccharin; or flavoring agents such as natural or synthetic oils.

Carriers for orally disintegrating tablets include for example lubricants, aggregating, sweetening, flavoring or disaggregating agents as well as agents improving the buccal mucosa absorption of Components (a) and (b), with optionally a further Component (c), such as sorbitol, mannitol, lactose and cellulose.

Carriers for liquid, normally aqueous, suspensions or solutions include for example antioxidants, such as sodium metabisulfite or sodium sulfite, thickening agents, such as microcrystalline cellulose, hydroxypropylcellulose, carboxymethylcellulose or polyvinylpyrrolidone, preservatives such as methyl paraben, ethyl paraben, sodium ethylenediaminotetracetate, sodium benzoate or an alkaline salt of sorbic acid, as well as flavoring and sweetening agents.

The sweeteners contained in the orally disintegrating tablets and the liquid suspensions or solutions may be natural, optional reduced sugars such as sucrose, dextrose, xylitol, mannitol or sorbitol, or synthetic product such as sodium saccharine or aspartame.

The flavoring agents are pharmaceutically acceptable flavors and tastes of synthetic and natural oils, the latter extracted from plants, leaves, flowers, fruits and their combinations, such as cinnamon, peppermint, anise and citron leaves, bitter almond, citrus fruits, in particular orange and/or lemon, linden and grapefruit oils. Also chocolate, vanilla or eucalyptus flavor and essences of fruit, in particular apple, pear, peach, strawberry, cherry, apricot, orange, lemon and grapes may be advantageously used.

The composition according to the present invention may be in form of a capsule containing two tablets as described herein above, one of them comprising Component (a) and the other comprising Component (b) in admixture with each other and with a pharmaceutical carrier. The unit form may also be a capsule containing two tablets as described herein above, one of them comprising Component (a), and the other comprising Component (b) with a pharmaceutical carrier. Component (c) may be optionally included in the composition as described herein above, with one or more of Components (a) and (b).

The unit form may also be a capsule containing two tablets as described herein above, one of them comprising Component (a) and the second comprising Component (b) in admixture with each other and with a pharmaceutical carrier. Component (c) may be optionally included in the unit form as described herein above, with one or more of Components (a) and (b).

The combination may be formulated in tablets in which one or both of the two components (a) and (b) is/are is in controlled-release formulation, for example as a dispersion of said component in hydroxypropyl methyl cellulose or in a film-coated microgranule. Advantageously, the M₂-antagonist, in an ER-formulation is in the core and the nsPAChA, in IR-formulation, is in the outer layer in multi-layer tablets in which, for example, both the core and the outer layer are coated with a film. Analogously, capsules made of two separated parts, one containing Component (a), in IR- or ER-formulation and the other containing Component (b), in IR- or ER-formulation, may be used. Component (d) may be optionally included in the combination as described herein above, with one or more of Components (a) and (b).

Carriers and vehicles for ER tablets include retardant materials such as acrylic and methacrylic acid polymers and copolymers; cellulose derivatives such as hydroxypropylmethylcellulose, hydroxyethylcellulose, hydroxypropylethylcellulose, hydroxypropylcellulose, methylcellulose, ethylcellulose, or sodium carboxymethylcellulose; gums; waxes; glycerides or aliphatic alcohols or a mixture thereof.

Component (a) and Component (b), with optionally a further Component (c), as the base thereof or as a pharmaceutically acceptable salt thereof, may also be formulated in a delivering transdermal pharmaceutical form, such as a patch, a gel, a cream, a spray, an ointment, a lotion or a paste, wherein Component (a), Component (b) or both the Components (a) and (b) are present in admixture with the common diluents and permeation enhancers. Component (c) may be optionally included in the TTS as described herein above, with one or more of Components (a) and (b).

The permeation enhancer may be any compound which allows the improved permeation of drugs through the skin (see for example the review in Pharmaceutical Technology, November 1997, pages 58-66, the disclosure of which is herein incorporated by reference in its entirety). Such substances may be lower (C₁-C₄) alkanols; fatty alcohols such as lauryl alcohol (dodecanol), alone or in combination with a lower alkanol; fatty acids such as linolenic acid or oleic acid; fatty acid esters such as isopropyl palmitate, stearate, linoleate, oleate or myristate; glycerol; glycerol monoesters such as glycerol monostearate, monolinoleate or monooleate; glycerol diesters; glycerol triesters such as triacetin; sucrose monostearate, monolinoleate or monooleate; sorbitan esters; fatty alcohol ethers having from 10 to 20 carbon atoms; glycols, such as diethylene glycol or propylene glycol; glycols lower alkyl ethers, such as diethylene glycol mono(C₂-C₄)alkyl ether, in particular diethylene glycol monoethyl ether.

These permeation enhancers are present in an amount from 0.01% to 20% by weight of the total weight of the composition, advantageously in an amount of from 0.05% to 10% by weight, preferably from 0.1% to 5% by weight

### The Pharmacological Assessment

### Example 1

### Experiment 1—Establishment of the Dose-Response to Otenzepad in a Mouse Model of Diarrhea

Male Swiss mice (4-6 weeks old), N=10 per treatment group were used, and treated with either vehicle (vehicle group) or increasing doses of otenzepad, a representative M₂-antagonist. Mice were randomly assigned to one of two experimental groups (vehicle; or increasing doses of otenzepad). Each animal was identified by its group name, cage number, series (day) of experiment, and number (1 to 10) written with permanent ink on the tail.
Mice were placed individually in cages without any bedding materials. During the experiment the number of fecal pellets was counted at different time-points, starting one hour before the time of the administration of the test compound (T0), as outlined below:

T−1 h to T0: counting of the accumulated fecal pellets excreted.

T0: administration of the test compound.

T0 to T+2 h: counting of the accumulated fecal pellets excreted.

T+2 h to T+4 h: counting of the accumulated fecal pellets excreted.

The total number of fecal pellets for each mouse was counted over time. An analysis of variance (ANOVA) was performed on the results. Fisher's Protected Least Significant Difference was used for pairwise comparisons; p values≤0.05 were considered significant. Grubbs' test (http://www.graphpad.com/quickcalcs/Grubbsl.cfm) was used to detect outliers for each parameter in each experimental group.
Results confirmed that otenzepad dose-dependently causes diarrhea.

### Experiment 2—Antagonism of Otenzepad-Induced Diarrhea in Mice by a Non-Selective Peripheral Muscarinic Receptor Antagonist

Male Swiss mice (4-6 weeks old), N=10 per treatment group were used. Animals were pretreated with solifenacin (a representative peripheral muscarinic receptor antagonist) or vehicle; 30 minutes later animals were treated with otenzepad at a dose that caused diarrhea (as determined in Experiment 1). The dose of solifenacin ordinarily ranged from 2 to 40 mg/kg.
Mice were placed individually in cages without any bedding materials. During the experiment the number of fecal pellets was counted at different time-points as outlined below:

T−1 h to T0: counting of the accumulated fecal pellets excreted.

T0: administration of solifenacin.

T30 min: administration of vehicle or otenzepad.

T 30 min to T 2.5 h: counting of accumulated fecal pellets excreted.

T+2.5 h to T+4.5 h: counting of accumulated fecal pellets excreted.

The total number of fecal pellets for each mouse was counted over time. An analysis of variance (ANOVA) was performed on the results. Fisher's Protected Least Significant Difference was used for pairwise comparisons. The p value≤0.05 were considered significant. Grubbs' test (http//www at graphpad.com/quickcalcs/Grubbs.cfm) was used to detect outliers for each parameter in each experimental group.
Results showed that solifenacin dose-dependently antagonized the diarrhea induced by otenzepad, thus confirming that the representative nsPAChA solifenacin suppresses the adverse effects of the representative M₂-antagonist otenzepad.

### Example 2

Evaluation of Cognition with Solifenacin and Otenzepad in the T-Maze Alternation Task in Mice
The T-maze continuous alternation task (T-CAT) is useful as model for studying compounds with cognitive enhancing properties. The T-maze consists of 2 choice arms and 1 start arm mounted to a square centre. Manual doors are provided to close specific arms during the force choice alternation task.
Male Swiss mice (4-6 weeks old), N=10 per treatment group were used, and were pre-treated with:
Solifenacin at the dose that blocked fecal pellet excretion in Experiment 2 of Example 1.
   Thirty minutes later mice were treated with either vehicle or one of two doses of otenzepad:
the highest dose that did not cause diarrhea;
a dose that caused diarrhea.
   Mice were randomly assigned to one of the different experimental treatment groups. Each animal was identified by its group name, cage number, series (day) of experiment, and number (1 to 10) written with permanent ink on the tail.
   The T-maze apparatus is made of gray Plexiglas with a main stem (55 cm long×10 cm wide×20 cm high) and two arms (30 cm long×10 cm wide×20 cm high) positioned at 90 degree angle relative to the main stem. A start box (15 cm long×10 cm wide) was separated from the main stem by a guillotine door. Horizontal doors were also provided to close specific arms during the force choice alternation task.
   The experimental protocol consisted of one single session, which started with 1 “forced-choice” trial, followed by 14 “free-choice” trials. In the first “forced-choice” trial, animals were confined for 5 seconds to the start arm and then were released while either the left or the right goal arm was blocked by the horizontal door.
   Animals then negotiated the maze, eventually entering the open goal arm, and returned to the start position. Immediately after the return of the animals to the start position, the left or right goal door was opened and the animals were allowed to choose freely between the left and right goal arm (“free choice trials). An animal was considered as having entered in arm when it placed its four paws in the arm. A session was terminated and animals were removed from the maze as soon as 14 free-choice trials had been performed or 10 min had elapsed, whichever event occurred first.
   The apparatus was cleaned between each animal using 40% ethanol. Urine and feces were removed from the maze. During the trials, animal handling and the visibility of the operator was minimized as much as possible.
   The percentage of alternation over the 14 free-choice trials was determined for each mouse and was used as an index of working memory performance. This percentage is defined as entry in a different arm of the T-maze over successive trials (i.e., left-right-left-right, etc.).
   Analysis of variance (ANOVA) was performed on the results. Fisher's Protected Least Significant Difference was used for pairwise comparisons; p values≤0.05 were considered significant. The drug-induced improvement of memory was calculated by setting the respective response of the saline/vehicle as 100% and that of the test group as 0% reversion. Grubbs' test (http://www.graphpad.com/quickcalcs/Grubbsl.cfm) was used to detect outliers for each parameter in each experimental group.
   Results showed that solifenacin did not affect cognitive response to otenzepad, and that response was higher with the higher dose of otenzepad, thus confirming that the representative M₂-antagonist otenzepad improves cognition in a dose-dependent manner also in the presence of the nsPAChA solifenacin.

### Use

As set forth herein above, Component (a) and Component (b) may be administered concurrently or sequentially to a patient suffering from a hypocholinergic disorder of the CNS such as Alzheimer type dementia and schizophrenia. In addition, a Component (c) an AChEI may be further administered with Component (a) and Component (b) as described herein.

In particular, Component (a) and Component (b) can be administered in a specific dosage regimen as illustrated above to treat Alzheimer type dementia, schizophrenia, schizophrenia associated dementia, and/or schizoaffective disorders.

Component (a) and Component (b) may also be administered simultaneously or sequentially to one another, in each case by the same or different administration route.

The combination of Component (a) and Component (b) such as in the same unit form, allow for the safe administration of high doses of Component (a) without dangerous adverse effects linked to the peripheral cholinergic action of said Component (a). Accordingly, the therapeutic efficacy of Component (a) of safely improve cognition of patients suffering from a hypocholinergic disorder of the CNS such as Alzheimer type dementia, schizophrenia, schizophrenia associated dementia, or schizoaffective disorders is enhanced, due to the combination of Component (b) with Component (a).

Thus, the present invention, in one aspect, provides a combination comprising or consisting essentially of, as Components:
(a) a muscarinic receptor antagonist selected from the group consisting of selective M₂-antagonists, and
(b) a muscarinic receptor antagonist selected from the group consisting of non-selective, peripheral anticholinergic agents (nsPAChAs);
   for use in the treatment of a hypocholinergic disorder of the CNS.

The M₂-antagonists used as Component (a), their properties and doses are described in “The M₂-antagonists” section above.

The nsPAChA used as Component (b), their properties and doses are described in “The nsPAChAs” section above.

For use, Component (a) and Component (b), together or separately, are formulated in pharmaceutical compositions prepared as described in “The Formulations” section above.

The present invention, in another aspect, provides a method for treating a hypocholinergic disorder of the CNS, which comprises administering to a patient in need of said treatment a combination comprising or consisting essentially of, as Components:
(a) a muscarinic receptor antagonist selected from the group consisting of selective M₂-antagonists, and
(b) a muscarinic receptor antagonist selected from the group consisting of non-selective, peripheral anticholinergic agents (nsPAChAs).

The method is carried out by administering Component (a) and Component (b) of said combination concurrently, or sequentially. Component (a) and Component (b) may be independently administered by oral or parenteral route, in particular by intramuscular or intravenous injection or by transdermal administration by a TTS such as a gel or a patch.

The M₂-antagonist used as Component (a), their properties and doses are described in “The M₂-antagonists” section above.

The nsPAChA used as Component (b), their properties and doses are described in “The nsPAChAs” section above.

For administering the combination to said patient, Component (a) and Component (b), together or separately, are formulated in pharmaceutical compositions prepared as described in the “Formulation” section above.

In the case of simultaneous administration of the two components, Component (a) and Component (b), in admixture with a pharmaceutical carrier or vehicle, may be associated in the same pharmaceutical composition, formulated as described in “The Formulations” section above, in a unit dose for oral or parenteral, including transdermal route according to known or conventional methods or technologies in the art. Component (c) an AChEI may be further administered with Component (a) and Component (b) as described herein.

In said method or use, the pharmaceutical compositions illustrated in the above sections will be administered once, twice or three times per day according to the condition of the patient and the severity of the disease.

### REFERENCES

Adis R&D Profile—“Otenzepad AFDX 116” Drug R&D 1999; Apr. 1(4):319-320.
Alcantara A A, Mrzljak L, Jakab R L, Levey A I, Hersch S M, Goldman-Rakic P S. “Muscarinic m1 and m2 receptor proteins in local circuit and projection neurons of the primate striatum: anatomical evidence for cholinergic modulation of glutamatergic prefronto-striatal pathway”. J Comp Neurol. 2001; Jun. 11; 434(4):445-60.
Anagnostaras S G 1, Murphy G G, Hamilton S E, Mitchell S L, Rahnama N P, Nathanson N M, Silva A J. “Selective cognitive dysfunction in ACh M1 muscarinic receptor mutant mice”. Nat Neurosci 2003; January; 6(1):51-58.
Birks J. “Cholinesterase inhibitors for Alzheimer's disease”. Cochrane Database Syst Rev. 2006; January; 25(1):CD005593.
Brashear H R 1997. Review of book “Muscarinic Agonists and the Treatment of Alzheimer Disease” (Edited by Abraham Fisher and R. G. Landes, 1996) in Neurology; 49.
Carey G J, Billard W, Binch H, 3rd, Cohen-Williams M, Crosby G, Grzelak M, Guzik H, Kozlowski J A, Lowe D B, Pond A J, Tedesco R P, Watkins R W, Coffin V L. “SCH 57790, a selective muscarinic M₂ receptor antagonist, releases acetylcholine and produces cognitive enhancement in laboratory animals”. Eur J Pharmacol 2001; 16:189-200.
Chackalamannil S., Davies R J, Wang Y, Asberom T, Doller D, Wong J, Leone D. “Total Synthesis of (+)-Himbacine and (+)-Himbeline”. J Org Chem 1999; 64(6):1932-1940.
Christensen E B, Andersen J B, Pedersen H, Jensen K G, Dalgaard L. “Metabolites of [(14)C]-5-(2-ethyl-2H-tetrazol-5-yl)-1-methyl-1,2,3, 6-tetrahydropyridine in mice, rats, dogs, and humans” Drug Metab Dispos 1999; November; 7(11):1341-1349.
Engel W W, Eberlein W G, Mihm G, Hammer R, Trummlitz G. “Tricyclic compounds as selective muscarinic receptor antagonists. 3. Structure-selectivity relationships in a series of cardioselective (M2) antimuscarinics”. J Med Chem 1989; 32(8):1718-1724.
Foster D J, Jones C K, Conn P J; “Emerging approaches for treatment of schizophrenia: modulation of cholinergic signaling”. Discov Med. 2012; December; 14(79):413-420.
Gao L, Waelrroeck M, Hofman S, Van Haver D, Milanesio M, Viterbo D, De Clercq P. “Synthesis and affinity studies of himbacine derived muscarinic receptor antagonists”. Bioorg Med Chem Lett. 2002; 12(15):1909-1912.
Greenlee W, Clader J, Asberom T, McCombie S, Ford J, Guzik H, Kozlowski J, Li S, Liu C, Lowe D, Vice S, Zhao H, Zhou G, Billard W, Binch H, Crosby R, Duffy R, Lachowicz J, Coffin V, Watkins R, Ruperto V, Strader C, Taylor L, Cox K, “Muscarinic agonists and antagonists in the treatment of Alzheimer's disease”. Il Farmaco 2001; April; 56(4):247-250.
http:// at site adisinsight.springer.com/search AQRA 741
Huebner C F, Donoghue E, Wenk P E. Nelson S J A. “A New Class of Highly Active Antihistaminics”. J Am Chem Soc 1960; 82(8):2077-2078.
Lachowicz J E, Duffy R A, Ruperto V, Kozlowski J, Zhou G, Clader J, Billard W, Binch H, 3rd, Crosby G, Cohen-Williams M, Strader C D, Coffin V: “Facilitation of acetylcholine release and improvement in cognition by a selective M2 muscarinic antagonist, SCH 72788”. Life Sci 2001; 68:2585-2592.
Lamping K G, Wess J, Cui Y, Nuno D W, Faraci F M. “Muscarinic (M) receptors in coronary circulation: gene-targeted mice define the role of M2 and M3 receptors in response to acetylcholine”. Arterioscler Thromb Vasc Biol. 2004; July, 24(7): 1253-1258.
Lazaris A, Cassel S, Stemmelin J, Cassel J C, Kelche C, “Intrastriatal infusions of methoctramine improve memory in cognitively impaired aged rats”. Neurobiol Aging 2003; March-April; 24(2):379-383.
Maggio R, Barbier P, Bolognesi M L, Minarini A, Tedeschi D, Melchiorre C., “Binding profile of the selective muscarinic receptor antagonist tripitramine”, Eur J Pharmacol 1994; Aug. 16; 268(3):459-462.
Melchiorre C, Cassinelli A, Quaglia W. “Differential blockade of muscarinic receptor subtypes by polymethylene tetraamines. Novel class of selective antagonists of cardiac M-2 muscarinic receptors”. J Med Chem. 1987; January; 30(1):201-204.
Melchiorre C, Bolognesi M L, Chiarini A, Minarini A, Spampinato S. “Synthesis and biological activity of some methoctramine-related tetraamines bearing a 11-acetyl-5,11-dihydro-6H-pyrido[2,3-b][1,4]-benzodiazepin-6-one moiety as antimuscarinics: a second generation of highly selective M2 muscarinic receptor antagonists”. J Med Chem 1993; 36, 3734-3737.
Minarini A, Quaglia W, Tumiatti V, Melchiorre C. “An improved synthesis of N,N′-bis[6-o-methoxyphenyl methyl hexylamino]-1,8-octanediamine (methoctramine)”. Chem Ind (London) 2 Oct. 1989, page 652.
Palani A, Dugar S, Clader J W, Greenlee W J, Ruperto V, Duffy R A, Lachowicz J E. “Isopropyl amide derivatives of potent and selective muscarinic M2 receptor antagonists”. Bioorg.MedChem Lett 2004; 14(7): 1791-1794.
Pfaff O, Hildebrandt C, Waelbroeck M, Hou X, Moser U, Mutschler E, Lambrecht G. “The (S)-(+)-enantiomer of dimethindene: a novel M2-selective muscarinic receptor antagonist”. Eur J Pharmacol 1995. Nov. 24; 286(3):229-240.
Pike B R, Hamm R J. “Post-injury administration of BIBN 99, a selective muscarinic M2 receptor antagonist, improves cognitive performance following traumatic brain injury in rats” Brain Res 1995; 686:37-43.
Quirion R, Wilson A, Rowe W, Aubert I, Richard J, Doods H, Parent A, White N, Meaney M J. “Facilitation of acetylcholine release and cognitive performance by an M(2)-muscarinic receptor antagonist in aged memory-impaired”. J Neurosci 1995 February; 15(2):1455-1462.
Rouse S T, Edmunds S M, Yi H, Gilmor M L, Levey A I. “Localization of M(2) muscarinic acetylcholine receptor protein in cholinergic and non-cholinergic terminals in rat hippocampus”. Neurosci Lett 2000 Apr. 28; 284(3):182-186.
Rowe W B, O'Donnell J P, Pearson D, Rose G M, Meaney M J, Quirion R. “Long-term effects of BIBN-99, a selective muscarinic M₂ receptor antagonist, on improving spatial memory performance in aged cognitively impaired rats”. Behav Brain Res 2003; 145:171-178.
Sramek J J, Forrest M, Mengel H, Jhee S S, Hourani J, Cutler N R. “A bridging study of LU 25-109 in patients with probable Alzheimer's disease”, Life Sci. 1998; 62(3):195-202.
Starck J-P, Talaga P., Quéré L, Collart P, Christophe B, Lo Brutto P, Jadot S, Chimmanamada D, Zanda M, Wagner A, Mioskowski C., Massingham R Guyaux M. “Potent anti-muscarinic activity in a novel series of quinuclidine derivatives”; Bioorg Med Chem Lett. 2006, 15 Jan.; 16(2):373-377.
Stoll C, Eltze M, Lambrecht G, Zentner J, Feuerstein T J, Jackisch R. “Functional characterization of muscarinic autoreceptors in rat and human neocortex”. J Neurochem. 2009 August; 110(3):837-847.
Thal L J, Forrest M, Loft H, Mengel H. “Lu 25-109, a muscarinic agonist, fails to improve cognition in Alzheimer's disease. Lu25-109 Study Group”. Neurology 2000 Jan. 25; 54(2):421-426.
Tzavara E T, Bymaster F P, Felder C C, Wade M, Gomeza J, Wess J, McKinzie D L, Nomikos G G. “Dysregulated hippocampal acetylcholine neurotransmission and impaired cognition in M2, M4 and M2/M4 muscarinic receptor knockout mice”. Mol Psychiatry, 2003; July, 8(7):673-679.
Wang Y, Chackalamannil S, Hu Z, McKittrick B A, Greenlee W, Ruperto V, Duffy R A, Lachowicz J E. “Sulfide analogues as potent and selective M2 muscarinic receptor antagonists”. Bioorg Med Chem Lett 2002; 12, 1087-1099.
Zhang W, Basile A S, Gomeza J, Volpicelli L A, Levey A I, Wess J. “Characterization of Central Inhibitory Muscarinic Autoreceptors by the Use of Muscarinic Acetylcholine Receptor Knock-Out Mice”. J Neurosci 2002 Mar. 1; 22(5):1709-1717.

1. A pharmaceutical combination comprising as Components:
(a) a muscarinic receptor antagonist selected from the group consisting of selective M₂-antagonists, and
(b) a muscarinic receptor antagonist selected from the group consisting of non-selective, peripheral anticholinergic agents (nsPAChAs).

2. The combination of claim 1, wherein said Component (a) is a selective M₂-antagonist selected from the group consisting of
5,11-dihydro-8-chloro-11-[[4-[3-[(2,2-dimethyl-1-oxopentyl)ethylamino]propyl]-1-piperidinyl]acetyl]-6H-pyrido[2,3-b][1,4]benzodiazepin-6-one (BIBN-99);
racemic 11-[[2-(Diethylamino)methyl]-1-piperidinyl]-acetyl]-5,11-dihydro-6H-pyrido[2,3-b][1,4]benzodiazepin-6-one (otenzepad);
dextrorotatory 11-[[2-(diethylamino)methyl]-1-piperidinyl]-acetyl]-5,11-dihydro-6H-pyrido[2,3-b]E1,4]benzodiazepin-6-one [(+)-otenzepad];
N-2-[2-[(dipropylamino)methyl]-1-piperidinyl]ethyl]-5-,6-dihydro-11-H-pyrido[2,3-b][1,4]benzodiazepine-11-carboxamide (AF-DX 384),
11-[[4-[4-(Diethylamino)butyl]-1-piperidinyl]acetyl]-5,11-dihydro-6H-pyrido[2,3-b][1,4]benzodiazepin-6-one (AQ-RA 741),
N,N-Dimethyl-3-[1-(2-pyridinyl)ethyl]-1H-indene-2-ethanamine (dimethindene)
N,N-Dimethyl-3-[(1S)-1-(2-pyridinyl)ethyl]-1H-indene-2-ethanamine [S-(+)-dimethindene];
N,N′-bis[6-[([2-methoxyphenyl)methyl]amino]hexyl]-1,8-octanediamine (methoctramine),
1,1,24-tris[[5,11-dihydro-6-oxo-6H-pyrido[2,3b][1,4]-benzodiazepin-11-yl)carbonyl]methyl]-8,17-dimethyl-1,8,17,24-tetraazatetracosane (tripitramine);
(3aR,4R,4aS,8aR,9aS)-4-{(E)-2-[(2R,6S)-1,6-dimethylpiperidin-2-yl]ethenyl}-3-methyldecahydronaphtho[2,3-c]furan-1(3H)-one (himbacine
(3S,3aR,4R,4aS,8aR,9aS)-3-Methyl-4-[2-((R)-1-methyl-6-(S)-methyl-piperidin-2-yl)-vinyl]-decahydro-naphtho[2,3-c]furan-1-one [(+)-himbacine];
(3aR,4R,4aS,8aR,9aS)-4-{(E)-2-[(2R,6S)-1,6-dimethylpiperidin-2-yl]ethynyl}-3-methyldecahydronaphtho[2,3-c]furan-1(3H)-one (himbacine analog);
4-cyclohexyl-alpha-[4[[4-methoxyphenyl]sulphinyl]-phenyl]-1-piperazineacetonitrile (SCH-57790);
4-[4-[1(S)-[4-[(1,3-benzodioxol-5-yl)sulfonyl]phenyl]ethyl]-3 (R)-methyl-1-piperazinyl]-4-methyl-1-(propylsulfonyl)piperidine (SCH-72788);
1′-(2-methylbenzoyl)-4-[[[(3,4-methylenedioxyphenyl)sulfonyl]phenyl]methyl]-1,4′-bipiperidine (SCH-76050);
1′-(2-amino-3-methylbenzoyl)-4-[[[(3-chlorophenyl)sulfonyl]phenyl]methyl]-1,4′-bipiperidine (SCH-211803);
1′-(2-amino-3-methylbenzoyl)-4-[[[(3-chlorophenyl)sulfonyl]phenyl]ethylenedioxy methyl]-1,4′-bipiperidine (SCH-217443);
1′-naphto-1-yl-4-[[4-[(methoxycarbonyl)methylthio]phenyl]methyl]-1,4′-bipiperidine (Wang Compound 30);
1′-(indol-4-yl)carbonyl-4-[[(4-isopropyl)carbonyl]phenyl]methyl]-1,4′-biperidine (Palani Compound 19);
1′-(indol-4-yl)carbonyl-4-[[(4-isopropyl)carbonyl]phenyl]ethylenedioxymethyl]-1,4′-biperidine (Palani Compound 30);
and pharmaceutically acceptable salts and solvates thereof.

3. The combination of claim 1 wherein said Component (a) is present in an amount of from 0.5 mg to 1500 mg.

4. The combination of claim 1, wherein said Component (b) is a nsPAChA selected from the group consisting of quaternary ammonium nsPAChAs, sulfonium nsPAChAs, (1S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)—N,N-diisopropyl-3-(2-hydroxy-5-methylphenyl)-3-phenylpropanamine (tolterodine) and its pharmaceutically acceptable salts, [2-[(1R)-3-(di(propan-2-yl)amino)-1-phenylpropyl]-4-(hydroxymethyl)phenyl] 2-methylpropanoate (fesoterodine) and its pharmaceutically acceptable salts.

5. The combination of claim 4 wherein said quaternary ammonium nsPAChAs or sulfonium nsPAChAs has the formula (I) 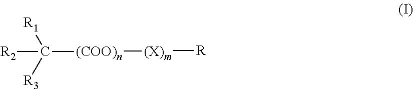
wherein
R is a radical selected from the group consisting of those of formulas (a)-(e) 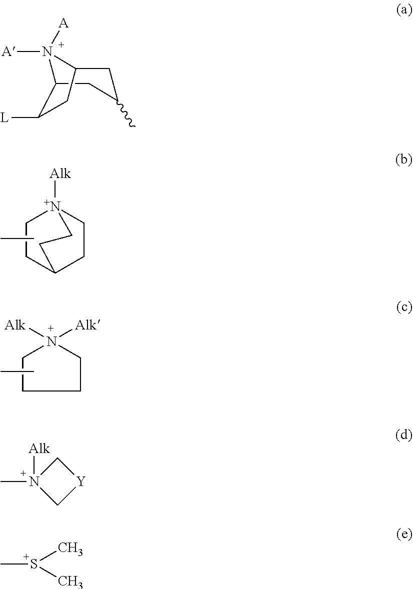
A being methyl and A′ being (C₁-C₄)alkyl or 2-fluoroethyl group or A and A′ forming a 1,4-butylene or 1,5-pentylene chain, L being hydrogen or methoxy, Alk and Alk′ each being (C₁-C₄)alkyl and Y being a bivalent radical selected from the group consisting of 1,2-ethylene, 1,3-propylene, 1,4-butylene and 2-oxa-1,3-propylene; the corresponding counter ion being a pharmaceutically acceptable anion;
n and m, independently, are zero or 1;
X is a (C₂-C₃)alkylene group;
R₁ and R₂ are each phenyl, cyclopentyl, cyclohexyl, 1-cyclohexenyl, 2-thienyl and, when R is a radical (a), also each represents (C₁-C₄)alkyl;
R₃ is H or OH or, only when R is a radical (a), also a COOAlk group, Alk being a (C₁-C₄)alkyl group.

6. The composition of claim 5, wherein, in said Formula I, at least one of m and n is 1.

7. The combination of claim 1, wherein said Component (b) is a nsPAChA selected from the group consisting of azoniaspiro[3β-benziloyloxy-(1α,5α)-nortropane-8,1′-pyrrolidine] (trospium) chloride, 3-[2-cyclopentyl(hydroxy)phenylacetoxy]-1,1-dimethylpyrrolidinium (glycopyrronium) bromide, solifenacin and the compound thereof with succinic acid (solifenacin succinate), propiverine and the hydrochloride thereof, oxyphencyclimine and the hydrochloride thereof, tolterodine and the hydrogen tartrate thereof, TTS-oxybutynin, fesoterodine and the fumarate thereof.

8. The combination according to claim 1 wherein said M₂-antagonist Component (a) is formulated in a pharmaceutical composition or device in admixture with a pharmaceutical carrier or vehicle.

9. The combination of claim 8, wherein said composition or device comprises a-nsPAChA Component (b) selected from the group consisting of quaternary ammonium nsPAChAs, sulfonium nsPAChAs, (1S)-(3R)-1-azabicyclo[2.2.2]oct-3-yl 3,4-dihydro-1-phenyl-2(1H)-iso-quinolinecarboxylate (solifenacin) and its pharmaceutically acceptable salts, 1-methylpiperidin-4-yl) 2,2-di(phenyl)-2-propoxyacetate (propiverine) and its pharmaceutically acceptable salts, 1,4,5,6-tetrahydro-1-methylpyrimidin-2-ylmethyl α-cyclohexyl-α-hydroxy-α-phenylacetate (oxyphencyclimine) and its pharmaceutically acceptable salts, (R)—N,N-diisopropyl-3-(2-hydroxy-5-methylphenyl)-3-phenylpropanamine (tolterodine) and its pharmaceutically acceptable salts, [2-[(1R)-3-(di(propan-2-yl)amino)-1-phenylpropyl]-4-(hydroxymethyl)phenyl] 2-methylpropanoate (fesoterodine) and its pharmaceutically acceptable salts.

10. A method for the therapeutic treatment of hypocholinergic type dementia, comprising administering to a patient in need of said treatment an effective dose of a muscarinic M₂ receptor antagonist, in combination with a non-selective, peripheral muscarinic anticholinergic agent (nsPAChA).

11. The method of claim 10, wherein the patient is suffering from Alzheimer type dementia.

12. A method for the therapeutic treatment of a hypocholinergic disorder, comprising administering to a patient in need of said treatment an effective dose of a muscarinic M₂ receptor antagonist, in combination with a non-selective, peripheral muscarinic anticholinergic agent (nsPAChA).

13. The method of claim 12, wherein the hypocholinergic disorder is selected from the group consisting of schizophrenia, schizophrenia associated dementia, and schizoaffective disorders.

14. The combination of claim 1, further comprising, as a Component, (c) an AChEI.
